(19) United States
(12) Patent Application Publication
(10) Pub. No.: US 2003/0082559 A1
(43) Pub. Date: May 1, 2003

(54) Methods and reagents for amplification and manipulation of vector and target nucleic acid sequences

(76) Inventors: David Beach, Boston, MA (US); Lisa Molz, Watertown, MA (US); Mark Caddle, Gloucester, MA (US)

Correspondence Address: ROPES & GRAY, ONE INTERNATIONAL PLACE, BOSTON, MA, 02110-2624, US

(21) Appl. No.: 10/055,719

(22) Filed: Jan. 22, 2002

Related U.S. Application Data
(60) Provisional application No. 60/262,937, filed on Jan. 19, 2001.
(60) Provisional application No. 60/269,591, filed on Feb. 16, 2001.

(51) Int. Cl. C12Q 1/68; C12P 19/34

(52) U.S. Cl. 435/6; 435/91.2

(57) ABSTRACT

The invention provides methods and compositions for the amplification of vector sequences, particularly for amplification of vectors applied to the elucidation of mammalian gene function. The present invention relates to methods and compositions for recovery/amplification of DNA sequences from mammalian complementation screening products, products of the functional inactivation of specific essential or non-essential mammalian genes, and products from the identification of mammalian genes which are modulated in response to specific stimuli. The methods and compositions of the present invention are applicable (but are not limited) to recovery of replication-deficient retroviral vectors, libraries comprising such vectors, retroviral particles produced by such vectors in conjunction with retroviral packaging cell lines, integrated provirus sequences derived from the retroviral particles of the invention and circularized provirus sequences which have been excised from the integrated provirus sequences of the invention. The compositions of the present invention further include novel retroviral packaging cell lines.

### REFERENCE TO RELATED APPLICATIONS

This application claims priority to U.S. Provisional Application 60/262,937, filed on Jan. 19, 2001, and U.S. Provisional Application 60/269,591, filed on Feb. 16, 2001, the entire contents of both applications are hereby incorporated herein by reference.

### BACKGROUND OF THE INVENTION

In yeast genetic systems, many options are available for delivery of gene sequences for the purpose of conferring a phenotype onto the host cell. For example, one common delivery system is a high copy plasmid system based on the endogenous yeast 2-micron plasmid. Plasmids from this origin achieve copy numbers of roughly 100 per cell and are randomly segregated to daughter cells upon division. In another system, the CEN system, CEN plasmids are maintained at low copy number (approximately 1 to 2 per cell) are segregated to daughter cells by the same mechanism used for segregation of the host chromosomes.

Further, methods have been devised in yeast by which the problems of gene isolation and discovery of gene function can be addressed efficiently. For example, in yeast it is possible to isolate genes via their ability to complement specific phenotypes. Further, in yeast, targeted insertional mutagenesis techniques can be used in yeast to inactivate or “knock out” a gene's activity. In mammalian systems, however, such methods are, in practical terms, lacking, which has made the elucidation of mammalian gene function a very difficult task.

For example, with respect to gene inactivation techniques in mammalian cells, the fact that mammalian cells are diploid and have complex genomes cause insertional mutagenesis techniques in mammalian systems to be a laborious, time-consuming and lengthy process.

Further, a major barrier to the development of such capabilities as complementation screening in mammalian cells has been that conventional techniques yield gene transfer efficiencies in most cells (0.01%-0.1%) that make screening of high complexity libraries impractical. While reports indicate that recombinant, replication deficient retroviruses can make possible increased gene transfer efficiencies in mammalian cells (Rayner & Gonda, 1994, Mol. Cell. Biol. 14: 880-887; Whitehead et al., 1995, Mol. Cell. Biol. 15:704-710), retroviral-based functional mammalian cloning systems are inconvenient and have, thus far, failed to achieve widespread use.

The lack of convenience and impracticality of current retroviral-based cloning systems include, for example, the fact that the production of high complexity libraries has been limited by the low transfection efficiency of known retroviral packaging cell lines. Furthermore, no system has provided for routine, easy recovery of integrated retroviral proviruses from the genomes of positive clones. For example, in currently used systems the recovery of retrovirus inserts may be accomplished by polymerase chain reaction (PCR) techniques, however this is quite time consuming and variable for different inserts. Furthermore, with the use of PCR, additional cloning steps are still required to generate viral vectors for subsequent screening. Additionally, no mechanism has been available for distinguishing revertants from provirus-dependent rescues, a major source of false positives.

For many years now, cultured mammalian cells have been the preeminent system for the production of therapeutic proteins. Consequently, the regulatory criteria applied to the use of cell culture systems is well established. At least partly for regulatory reasons, two cell systems have become the accepted standard for protein production: CHO cells and a myeloma cell line, NS0. Both of these cell lines have been adapted for growth in suspension in large-scale culture and for growth in serum-free media.

The use of mammalian cell culture systems is well suited to therapeutic protein production. Proteins synthesized in mammalian cells have a high probability of being correctly folded and of having the same post-translational modifications found in native proteins. With some effort and significant capital investment, mammalian cell culture can be scaled to industrial levels. In theory, the same engineered cell line can produce protein in a 15-liter or 15,000-liter fermentor. Thus, despite the emergence of new and promising technologies for large-scale protein production, mammalian cell culture is likely to remain a dominant method for protein manufacturing for the foreseeable future. However, continuous improvement of this well-established technology will be required to keep pace with the demand for faster and more efficient methods for producing novel biotherapeutics and to remain competitive with alternative approaches.

The key to successful production of therapeutic proteins in cultured cell is the creation of cell lines that express the desired protein product to high levels and retain stable levels of expression. Current practice relies on stepwise, drug-selected gene amplification to create cell lines where an expression cassette is present at a high-copy number. Increased representation of the expression unit correlates well with increased expression of the desired product. Two routes are commonly applied to accomplish this goal. In dihydrofolate reductase (DHFR)-deficient CHO cells, an expression cassette encoding the therapeutic protein is delivered by transfection in combination with the DHFR gene. Stepwise treatment with increasing levels of methotrexate can, over a course of months, select for cells in which the DHFR gene has been amplified. Often the desired expression cassette is coamplified, resulting in up to a 20-fold increase in the level to which a therapeutic protein is expressed. An alternative but equivalent system is commonly utilized in both CHO and NS0 cells. This approach relies on amplification of sequences encoding glutamine synthetase upon treatment with methionine sulphoximine. Although both approaches can effectively net gene amplification and increases in protein expression, their application is a long process. Serial selection in increasing drug concentrations often proceeds over a period of six or more months.

At the end of this process a large number of individual cell lines must be tested for protein expression to identify those that are suitable for large-scale production. This process is so labor intensive that a suboptimal cell line will often be used to begin production while the search for more efficient producer cells continues. Identification of such cells often necessitates a late-stage exchange of cell lines that requires another round of FDA approval. Furthermore, the net result of drug-selected gene amplification is an array consisting of repeats of the expression unit. This is an inherently unstable situation, and it is well established that, in the absence of selective pressure, these repeats tend to evolve toward unit copy number. This problem becomes significant because drug selection cannot be maintained as cell lines are used for large-scale protein production. Thus, each protein production run is a gamble on whether the particular cell line has significantly reduced the extent of the direct repeats early in the run. Additionally, the instability of the repeat structure limits the viable length of the production run. Currently, the standard approach to large-scale protein production involves fixed-term batch culture; however, as the demand for therapeutic proteins increases, a shift to fed-batch, continuous, or perfusion cultures is likely to produce significant increases in efficiency provided that stability problems can be overcome.

Further, it would be advantageous if an episomal system such as those found in yeast existed for efficient, broad spectrum use in mammalian systems. While bovine papillomaviruses (BPV), for example, replicate as extrachromosomal episomes, their use in developing episomal vectors has been limited.

Specifically, the ability of BPV replicate as episomes has been exploited in the past to create episomal vectors, using the so-called 69% fragment (T69). Vectors based upon T69 replicate in certain murine cell lines to give copy numbers that range from 15 to 500 copies per haploid genome, depending on the cell line. T69 vectors, however, exhibit a narrow host range. Further, the T69 fragment, like SV40, is oncogenic. Indeed, one method for identifying cells carrying T69 vectors specifically involves screening for transformed C127 cells.

The amplification and recovery of mammalian cloning vectors provides a powerful tool for genetic engineering. Numerous advances in the art of polymerase-mediated amplification have been developed and may be applied in the method of the present invention.

### SUMMARY OF THE INVENTION

The present invention relates to methods and compositions for the elucidation of mammalian gene function. Such methods can utilize novel integrating and/or episomal genetic delivery systems, thereby providing flexible, alternate genetic platforms for use in a wide spectrum of mammalian cells, including human cells. The invention relates to methods and compositions for improved mammalian complementation screening, functional inactivation of specific essential or non-essential mammalian genes, identification of mammalian genes which are modulated in response to specific stimuli, identification of mammalian genes that encode secreted products, and production and selection of novel retroviral packaging cell lines.

Specifically, the invention relates to methods for amplification, detection and/or recovery of such vectors. In particular, the invention provides useful methods for amplifying an integrated retroviral vector. Amplification of such a target nucleic acid sequence integrated into a target cell genome is achieved by excising the target nucleic acid sequence from the target cell genome; amplifying the excised target nucleic acid sequence by rolling circle amplification with a polymerase to generate a tandem series of the target nucleic acid sequence; and excising the target nucleic acid sequence from the tandem series to produce individual target nucleic acid sequences. In certain instances, the method of the invention may optionally include a pre-amplification of the entire target cell genome prior to excising the target nucleic acid sequence from the target cell genome. Preferably, the target nucleic acid sequence is a replication-deficient retroviral vector described herein and the target cell genome is a mammalian genome. In certain embodiments the target cell genome is pre-amplified by whole genome amplification or by mitotic division of the viable mammalian cell encompassing the targeted genome.

Excision of the target nucleic acid from the target cell genome is effected by recombination with a recombinase, preferably a Kw recombinase, or, alternatively, by restriction with a restriction endonuclease that cuts within the target nucleic acid sequence to release a target nucleic acid sequence with ligatable ends, which are optionally ligated to effect circularization of the target sequence. In certain embodiments, the restriction endonuclease employed is an HO endonuclease. Thee target nucleic acid sequence may be amplified by a factor of at least about 10, and, more preferably, by a factor of at least about 100, 1,000, 10,000, 100,000 or 1,000,000. In preferred embodiments, the target nucleic acid sequence excised from the target cell genome is amplified by rolling circle amplification using a Phi 29 DNA polymerase, although the use of other DNA polymerases, particularly DNA polymerases from double-stranded DNA viruses, is within the scope of the invention. In preferred embodiments, the target nucleic acid sequence excised from the target cell genome is amplified by rolling circle amplification using a polymerase and the target nucleic acid sequence is a retroviral vector having long terminal repeat ends.

In another aspect, the invention provides methods for mobilizing a library of target nucleic acid sequence from one vector system to another. This method may be applied to individual clones as well so as to the transfer of an individual target sequence from one vector to another. In preferred embodiments, a complex mixture of target nucleic acid sequences, such as a cDNA library, is mobilized. In general, the mobilization of the target nucleic acid sequence(s) is effected by first excising the target nucleic acid sequence(s) from the first vector system, and then amplifying the excised target nucleic acid sequence(s) by rolling circle amplification with a polymerase to generate a tandem series of the target nucleic acid sequence(s). Preferably, the target nucleic acid sequence excised from the first vector system is amplified by rolling circle amplification using a Phi 29 DNA polymerase, however the use of any DNA polymerase with strand displacement activity is within the scope of the invention. In certain embodiments, a DNA polymerase from a double stranded DNA virus is envisioned. When the starting material is a cDNA library or other mixture of different target nucleic acid sequences, then the excised sequences represent a mixture of amplified target nucleic acid sequences with free ligatable ends. When the starting material is an individual cDNA clone, then the excised sequence represents a single species of amplified target nucleic acid sequence with free ligatable ends. The resulting liberated target nucleic acid sequences are then ligated into a second vector system via their compatible free ends. The overall effect is thus to transfer the original individual or mixture of target nucleic acid sequence(s) from the first vector system to a second different vector system. This methodology may be employed with any vector system, however, in general, preferred vector systems include the replication-deficient retroviral vector systems of the invention. Accordingly, preferred target nucleic acid sequences are a part of a retroviral target sequence having long terminal repeats.

In yet another aspect, the method of the invention provides a method of converting a mixture of partial cDNA target nucleic acid sequences to a mixture of cognate full-length cDNA target nucleic acid sequences. This aspect of the invention provides a generally convenient means of converting one form of library into another form of library representing related sequences. For example, a cDNA library could be conveniently converted into a genomic DNA library. In a preferred embodiment, a partial cDNA library, such as an EST or a secretion trap library, can be converted into a corresponding full-length cDNA library in which each species of the original partial cDNA mixture has a corresponding species in the full-length cDNA library—i.e. a cognate full-length clone derived from the same gene as the partial clone. In general, the conversion of the target nucleic acid sequence(s) is effected by first excising the partial target nucleic acid sequence(s) from the first vector system, and then amplifying the excised partial target nucleic acid sequence(s) by rolling circle amplification with a polymerase to generate a tandem series of the target nucleic acid sequence(s). In this embodiment, the rolling circle amplification is preferably performed with a single primer so that only a single-stranded copy of each target nucleic acid probe is created. This single-stranded tandem series of partial target nucleic acid sequences is then used as a hybrid capture probe for selecting the corresponding cognate full-length nucleic acid sequences. The selected full-length sequences are then released from the hybrid capture probe(s), e.g. by elution—for example, under denaturing conditions. The library of full-length nucleic acid sequences is provided as either a single stranded library, such as an M13 phagemid library, or as a denatured double-stranded library. In either case, the hybrid capture probe(s) select out the corresponding full-length sequence(s). The overall effect of the method is to thereby produce a full-length target nucleic acid sequence from each of the starting partial target nucleic acid sequences. Notably, depending upon the nature of the first and second vector system, the nature of the vector can also be changed. For example, a starting partial cDNA library from a secretion trap library can be converted into a full-length cDNA library based upon one of the retroviral expression vectors of the invention. If the starting material is a single species of target nucleic acid sequence, the product is a corresponding (cognate) full-length species. If the starting material is a collection of target nucleic acid sequences, such as a partial cDNA or EST library, the product is a corresponding library of full-length target nucleic acid sequences.

The compositions of the present invention include, but are not limited to, replication-deficient retroviral vectors, libraries comprising such vectors, retroviral particles produced by such vectors in conjunction with retroviral packaging cell lines, integrated provirus sequences derived from the retroviral particles of the invention and circularized provirus sequences which have been excised from the integrated provirus sequences of the invention.

The compositions of the present invention further include ones relating to improved mammalian episomal vectors. In particular, these compositions include, but are not limited to, expanded host range vectors (pEHRE), and libraries, cells and animals containing such vectors. The pEHRE vectors of the invention provide a consistent, stable, high-level episomal expression of gene sequences within a broad spectrum of mammalian cells. The pEHRE vectors of the invention comprise, first, replication cassettes in which papillomavirus (PV) E1 and E2 proteins are expressed from a constitutive transcriptional regulatory sequence or sequences, and, second, minimal cis-acting elements for replication and stable episomal maintenance.

The pEHRE vectors include, but are not limited to, vectors for delivery of sense and antisense expression cassettes, regulated expression cassettes, large chromosomal segments, and cDNA libraries, to a wide range of mammalian cells. Among the pEHRE vectors presented are ones which, additionally, can be utilized for the large scale production of recombinant proteins, and ones which can be utilized in the construction of cell lines that stably produce high titer viruses.

The compositions of the present invention further include novel viral packaging cell lines. In particular, described herein are novel, stable retroviral packaging cell lines which efficiently package retroviral-derived nucleic acid into replication-deficient retroviral particles capable of infecting appropriate mammalian cells. Such packaging cell lines are produced by a novel method which directly links the expression of desirable viral proteins with expression of a selectable marker.

The retroviral packaging cell lines of the invention provide retroviral packaging functions as part of a polycistronic message which allowing direct selection for the expression of such viral functions and, further, makes possible a quantitative selection for the highest expression of desirable sequences.

In particular, the methods of the present invention include, but are not limited to, methods for the identification and isolation of nucleic acid molecules based upon their ability to complement a mammalian cellular phenotype, antisense-based methods for the identification and isolation of nucleic acid sequences which inhibit the function of a mammalian gene, gene trapping methods for the identification and isolation of mammalian genes which are modulated in response to specific stimuli, methods for efficient large scale recombinant protein expression and methods for modulating the expression of known genes.

Thus, one aspect of the invention provides a method of amplifying a target nucleic acid sequence in a target cell genome comprising: (A) excising the target nucleic acid sequence from the target cell genome; (B) amplifying the excised target nucleic acid sequence by rolling circle amplification with a polymerase to generate a tandem series of the target nucleic acid sequence; and, (C) excising the target nucleic acid sequence from the tandem series to produce individual target nucleic acid sequences; thereby amplifying the target nucleic acid sequence in the target cell genome.

In one embodiment, the method further comprises amplifying the entire target cell genome prior to excising the target nucleic acid sequence from the target cell genome. In a preferred embodiment, the entire target cell genome is amplified by whole genome amplification. In a more preferred embodiment, the entire target cell genome is in a cell and the entire target cell genome is amplified by mitotic division of said cell.

In another embodiment, the target nucleic acid sequence is a replication-deficient retroviral vector.

In another embodiment, the target cell genome is a mammalian genome.

In another embodiment, excision of the target nucleic acid from the target cell genome is effected by recombination with a recombinase. The recombinase can be a Kw recombinase.

In another embodiment, excision of the target nucleic acid from the target cell genome is effected by restriction with a restriction endonuclease that cuts within the target nucleic acid sequence to release a target nucleic acid sequence with ligatable ends. In a preferred embodiment, the method further comprises ligating said ends. In a preferred embodiment, the endonuclease is an HO endonuclease.

In another embodiment, the target nucleic acid sequence is amplified by a factor of at least about 10, more preferably by a factor of at least about 100, 1,000, 10,000, 100,000, or most preferably, by a factor of at least about 1,000,000.

In another embodiment, the target nucleic acid sequence excised from the target cell genome is amplified by rolling circle amplification using a Phi 29 DNA polymerase.

In another embodiment, the target nucleic acid sequence excised from the target cell genome is amplified by rolling circle amplification using a DNA polymerase derived from a double stranded DNA virus.

In another embodiment, the target nucleic acid sequence is a retroviral vector having long terminal repeat ends.

Another aspect of the invention provides a method of transferring a mixture of target nucleic acid sequences from a first vector system to a second vector system comprising: (A) providing a first library of target nucleic acid sequences in a first vector system; (B) excising the target nucleic acid sequences from the first vector system; (C) amplifying the excised target nucleic acid sequences by rolling circle amplification with a polymerase to generate tandem series of the target nucleic acid sequences; (D) excising the target nucleic acid sequences from the amplified tandem series of the target nucleic acid sequences to generate a mixture of amplified target nucleic acid sequences with free ends; (E) providing a second vector system compatible with said free ends; and, (F) ligating the free ends of the amplified and/or excised target nucleic acid sequences to the second vector system, thereby transferring a mixture of target nucleic acid sequence form the first vector system to the second vector system.

In one embodiment, the first vector system is a replication-deficient retroviral vector.

In another embodiment, the target nucleic acid sequence excised from the first vector system is amplified by rolling circle amplification using a Phi 29 DNA polymerase.

In another embodiment, the target nucleic acid sequence excised from the first vector system is amplified by rolling circle amplification using a DNA polymerase derived from a double stranded DNA virus.

In another embodiment, the target nucleic acid sequence is a retroviral vector having long terminal repeat ends.

Another aspect of the invention provides a method of converting a mixture of partial cDNA target nucleic acid sequences to a mixture of cognate full-length cDNA target nucleic acid sequences comprising: (A) providing a first library of partial cDNA target nucleic acid sequences in a first vector system; (B) excising the partial cDNA target nucleic acid sequences from the first vector system; (C) amplifying the excised target nucleic acid sequences by rolling circle amplification to generate a hybrid capture probe mixture; (D) contacting the hybrid capture probe mixture with a second library of full-length single-stranded cDNA target nucleic acid sequences to select full-length single-stranded cDNA sequences which correspond to the partial cDNA target nucleic acid sequences of said first library; and, (E) releasing the selected full-length single-stranded cDNA sequences from the second library; thereby converting a mixture of partial cDNA target nucleic acid sequences to a mixture of cognate full-length cDNA target nucleic acid sequences.

Another aspect of the invention provides a method of converting a mixture of cDNA target nucleic acid sequences to a mixture of cognate genomic target nucleic acid sequences comprising: (A) providing a first library of cDNA target nucleic acid sequences in a first vector system; (B) excising the cDNA target nucleic acid sequences from the first vector system; (C) amplifying the excised cDNA target nucleic acid sequences by rolling circle amplification to generate a hybrid capture probe mixture; (D) contacting the hybrid capture probe mixture with a second library of cognate genomic target nucleic acid sequences to select cognate genomic sequences which correspond to the cDNA target nucleic acid sequences of said first library; and, (E) releasing the selected cognate genomic target nucleic acid sequences from the second library; thereby converting a mixture of cDNA target nucleic acid sequences to a mixture of cognate genomic target nucleic acid sequences.

Another aspect of the invention provides a method of amplifying polynucleotides, comprising amplifying the polynucleotides by rolling circle amplification with a polymerase and random primers.

In one embodiment, the polynucleotide is genomic DNA (gDNA). The genomic DNA can be whole genomic DNA isolated from cells.

In another embodiment, the polynucleotide is cDNA. The cDNA can be reverse transcribed from RNA. In a preferred embodiment, the method further comprises a step of size-fractionating the resulting amplified cDNA to select for substantially full-length cDNA.

In another embodiment, the random primers are random hexamers.

In another embodiment, the polymerase is Phi 29 DNA polymerase.

Another aspect of the invention provides a method to clone a DNA fragment from a single cell, comprising: A) isolating genomic DNA containing the DNA fragment; B) amplifying the isolated genomic DNA by rolling circle amplification using a polymerase and primers; C) excising the DNA fragment from the amplified genomic DNA; and, D) cloning the excised DNA fragment.

In one embodiment, the DNA fragment is a provirus.

In another embodiment, the primers are random hexamers.

In another embodiment, the polymerase is Phi 29 DNA polymerase.

In another embodiment, DNA fragments are excised by restriction endonuclease.

In another embodiment, the method further comprises colonal expansion of the single cell prior to isolating genomic DNA.

In another embodiment, the DNA fragments are flanked by recombinase recognition sites and the DNA fragments are excised by a corresponding recombinase of a site specific recombinase system. The recombinase can be Kw recombinase. In a preferred embodiment, the site specific recombinase system is selected from the group consisting of: the Cre/lox system of bacteriophage P1, the FLP/FRT system of yeast, the Gin recombinase of phage Mu, the Pin recombinase of E. coli, and the R/RS system of the pSR1 plasmid.

In another embodiment, the method further comprises a step to enrich the excised DNA fragment prior to cloning.

Another aspect of the invention provides a kit for amplifying a polynucleotide, comprising: A) a DNA polymerase suitable for rolling circle amplification; B) a reaction buffer for carrying out rolling circle amplification using the DNA polymerase; C) a mixture of random hexamer oligonucleotides.

In one embodiment, the method further comprises at least one of the components selected from the group consisting of: an instruction for using the kit; a control polynucleotide; a stock solution of dNTP mixtures or each of the four deoxynucleotides (dATP, dGTP, dCTP and dTTP).

Another aspect of the invention provides a polynucleotide sequence comprising a vector as shown in any one of FIGS. 1-23, or derivative thereof.

Another aspect of the invention provides a polynucleotide sequence comprising a reunification vector as shown in FIG. 25 or derivative thereof

Another aspect of the invention provides a library comprising any one of the vector of the instant invention.

The present invention is related in part to the disclosures made in WO 98/12339, the specification of which is herein incorporated by reference.

### BRIEF DESCRIPTION OF THE FIGURES

FIG. 1. The arrangement of DNA elements that comprise the replication-defective retroviral vector, MaRX II. psi denotes the packaging signal.

FIG. 2. Diagrammatic representation of the cleavage of the loxP sites with Cre recombinase enzyme, yielding an excised provirus which upon excision, becomes circularized.

FIG. 3. The arrangement of DNA elements that comprise the retroviral vector for expression/sense complementation screening, p.hygro.MaRX II-LI.

FIG. 4. The arrangement of DNA elements that comprise a retroviral vector for peptide display, pMODis-I.

FIG. 5. The arrangement of DNA elements that comprise a retroviral vector for peptide display, pMODis-II.

FIG. 6. The arrangement of DNA elements that comprise the retroviral vector for gene trapping, pTRAP II.

FIG. 7. The arrangement of DNA elements that comprise a retroviral vector for antisense complementation screening, pMaRX IIg.

FIG. 8. The arrangement of DNA elements that comprise a retroviral vector for antisense complementation screening, pMaRX IIg-demV.

FIG. 9. The arrangement of DNA elements that comprise a retroviral vector for antisense complementation screening, pMaRX IIg-va.

FIG. 10. The arrangement of DNA elements that comprise a pEHRE vector for expression/sense complementation screening, pEHRE-E-H.

FIG. 11. The arrangement of DNA elements that comprise a pEHRE vector for large scale protein production, pEHRE-H.

FIG. 12. The arrangement of DNA elements that comprise a pEHRE vector for use in production of pEHRE/BAC hybrid constructs, pBPV-BacDonor.

FIG. 13. The arrangement of DNA elements that comprise a pEHRE vector for use as a BAC cloning vector.

FIG. 14. The arrangement of DNA elements that comprise a pEHRE antisense GSE vector, pEHRE-GSE-H.

FIG. 15. The arrangement of DNA elements that comprise a pEHRE antisense GSE vector, pEHRE-GSEVA-H.

FIG. 16. The arrangement of DNA elements that comprise a pEHRE antisense GSE vector, pEHRE-GSEU6-H.

FIG. 17. The arrangement of DNA elements that comprise a pEHRE vector for packaging cell line use, ψ_{c}IH.

FIG. 18. The arrangement of DNA elements that comprise a pEHRE vector for packaging cell line use, pEHRE-ψ_{c}IH.

FIG. 19. The arrangement of DNA elements that comprise a pEHRE vector for packaging cell line use, ψₑₙᵥIH.

FIG. 20. The arrangement of DNA elements that comprise a pEHRE vector for packaging cell line use, pEHRE-ψₑₙᵥIH.

FIG. 21. The arrangement of DNA elements that comprise a pEHRE vector for packaging cell line use, ψ_{g/p}IH.

FIG. 22. The arrangement of DNA elements that comprise a pEHRE vector for packaging cell line use, pEHRE-ψ_{g/p}IH.

FIG. 23. The arrangement of DNA elements that comprise a representative retroviral secretion trapping vector.

FIG. 24. A graph showing the relative stability of the linX packaging cell line as compared to the Phoenix and bosc23 cell lines.

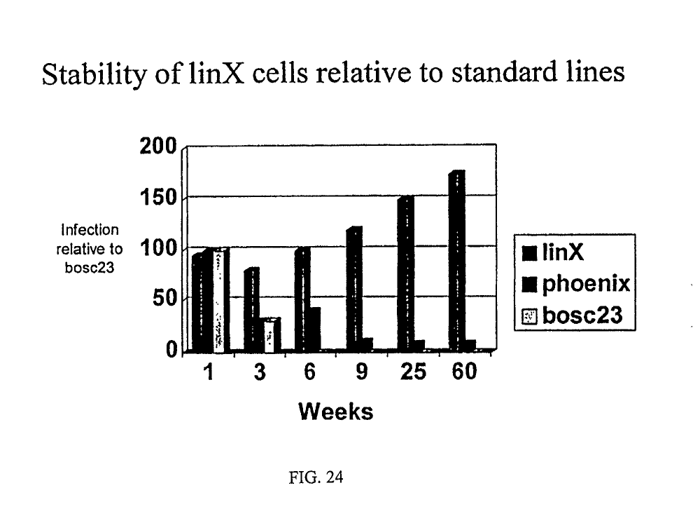

FIG. 25. An exemplary use of the reunification plasmid to restore LTR elements to excised proviral vectors.

### DETAILED DESCRIPTION OF THE INVENTION

4.1. General

The invention provides new and useful methods for the amplification and amplification-mediated recovery of DNA, particularly recombinant genetic material such as expression cloning vectors. Expression cloning of cDNAs using mammalian cells has been a long sought after goal in molecular biology. It is a potentially a powerful tool with which to isolate a nucleic acid of interest, such as a cDNA, under circumstances wherein a phenotypic function of a protein is known but its amino acid sequence is not known. For instance, many growth factor and cytokine genes were cloned by scoring for a growth-promoting activity of culture supernatant of COS cells transiently transfected with expression vectors engineered with cDNA libraries.

Many expression cloning systems of the prior art work on the principle of amplifying expression vectors carrying the SV40 replication origin (SV40 ori) in mammalian cells stably expressing T antigen (i.e., a transformed African green monkey kidney cell line, COS). The presence of the SV40 large T antigen in COS cells allows replication of SV40 ori containing plasmids, thus amplifying expression of the cDNA on the plasmid.

Despite many successful applications, conventional expression cloning systems still suffer from the need for transient amplification of plasmids in particular cell lines expressing the SV40 (or polyoma) large T antigen. First of all, the function of the target gene has to be suited to transient detection. Moreover, target cells are restricted to those which allow SV40 large T antigen-based amplification and to those cell types in which the transfection efficiency is high (e.g., greater than 10%). Approaches using transient expression system in COS cells or fibroblasts have obvious limitations in searches for proteins with various functions in various types of cells.

To overcome these limitations, one aspect of the present invention relates to high efficiency viral expression cloning systems. In one embodiment, the subject expression constructs are generated using viral vectors which can be stably integrated into the genome of a metazoan host cell, particularly a mammalian host cell. To illustrate, in one embodiment a preferred viral expression construct is derived from a retroviral vector which, in addition to being capable of expressing a heterologous gene when integrated in the host cell, also includes one or more various other features including, e.g., means for excising the retroviral vector from the genome of the host cell, means for recovering the excised vector, and/or means for amplifying or otherwise manipulating the vector in prokaryotic cells. Other variations are described more fully below.

To further illustrate, the subject viral vectors can be engineered with a nucleic acid library of interest, and as appropriate, infectious particles produced. For packaging into viral particles, viral packaging system known in the art can be used, or, more preferably, the viral vectors can be packaged with the novel transient packaging system described herein. The engineered virus is than used to infect a selected host cell. The infected cells can subsequently be screened for expression of nucleic acid of interest, e.g., based on a change in phenotype of the cell.

According to the present invention, expression cloning systems based on high complexity viral libraries can allow investigatory access to many important cell types and cell signaling systems not previously accessible by prior techniques. The subject viral cDNA library transfer approaches offer numerous advantages to those interested in complementation cloning in, for example, mammalian cells. For instance, in contrast to transient transfection of plasmids, gene transfer with such viral vectors as, for example, the exemplary retroviruses and adeno-associated viruses, can deliver genes stably into a wide range of target cells. This feature helps to overcome a disadvantage of conventional transient gene expression for phenotypic selection by extending the amount of time over which the phenotypic change can be observed.

Moreover, the use of the subject viral vectors can also overcome another major limitation in the art, that of generally low transfection rates which otherwise makes adequate representation of genes in complex nucleic acid libraries difficult. In contrast to transfection, the subject virus can efficiently infect and transfer genes to a wide range of cells.

Thus, the power of complementation cloning, long appreciated in bacterial and yeast genetic systems, may now be more fully accessed for mammalian and other metazoan cells by the viral-based approaches we describe herein.

As described with greater detail below, such compositions of the present invention include, but are not limited to, replication-deficient retroviral vectors, libraries comprising such vectors, retroviral particles produced by such vectors in conjunction with retroviral packaging cell lines, integrated provirus sequences derived from the retroviral particles of the invention and circularized provirus sequences which have been excised from the integrated provirus sequences of the invention. Similar compositions derived using viral sequences for other genomically-incorporated viruses are also specifically contemplated, including vectors based on the adeno-associated virus (AAV).

Yet another aspect of the present invention relates to episomal expression vectors which also can be used to overcome certain of the above-described deficiencies in the mammalian expression cloning systems of the art. In particular, the compositions of the invention described herein further include improved mammalian episomal vectors as well as libraries, cells and animals containing such vectors. The compositions of the present invention described herein still further include novel viral, including retroviral, packaging cell lines.

Second, the methods of the invention are described. Such methods include, but are not limited to, methods for the identification and isolation of nucleic acid molecules which complement a mammalian cellular phenotype, antisense-based methods for the identification and isolation of nucleic acid sequences which inhibit the function of a mammalian gene, gene trapping methods for the identification and isolation of mammalian genes which are modulated in response to specific stimuli, methods for the identification of mammalian genes that encode secreted proteins, methods for the selection and production of novel viral packaging cell lines and methods for efficient large scale recombinant protein expression.

The methods of the present invention also include, but are not limited to, methods for the identification and isolation of peptide sequences by complementation type screens using vectors capable of displaying random or semi-random peptide sequences which will interact with proteins important for a particular cellular or viral function. This interaction can result in, e.g., the elaboration of selectable phenotype.

Still another aspect of the invention relates more generally to vectors derived with transposable elements and proviral excision element. The transposable elements can be selected from amongst any of a variety class of nucleic acid elements capable of movement from one position to another in the genome. For instance, the transposable element can be derived from, e.g., retroviral LTRs as described above, or can be any other form of transposon or retrotransposon which gives rise to an integrative vector. In preferred embodiments, the invention takes advantage of certain properties of the phage Phi 29 DNA polymerase.

4.2. Definitions

For convenience, certain terms employed in the specification, examples, and appended claims are collected here.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

A “chimeric protein” or “fusion protein” is a fusion of two amino acid sequences of heterologous origin, by generating a chimeric coding sequence in which the coding sequences for the first and second polypeptide are fused in frame so as to produce, upon initial translation, a single polypeptide chain.

A “coding sequence” or a sequence which “encodes” a particular polypeptide, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

By a “DNA binding domain” or “DBD” is meant a polypeptide sequence which is capable of directing specific polypeptide binding to a particular DNA sequence (i.e., to a DBD recognition element). The term “domain” in this context is not intended to be limited to a discrete folding domain. Rather, consideration of a polypeptide as a DBD for use in the bait fusion protein can be made simply by the observation that the polypeptide has a specific DNA binding activity. DNA binding domains, like activation tags, can be derived from proteins ranging from naturally occurring proteins to completely artificial sequences.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence. On the other hand, “expression” of an antisense sequence or ribozyme will be understood to refer to the transcription of the recombinant gene sequence.

As used herein, the term “gene” or “recombinant gene” refers to a nucleic acid which is transcribed and (optionally) translated. Thus, a recombinant gene can comprise an open reading frame encoding a polypeptide, including both exon and (optionally) intron sequences. In other embodiments, a recombinant gene can simply provide, on transcription, an antisense transcript, a ribozyme, or other RNA molecule for which the effect of transcription on the phenotype of the cell is to be scored.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct is a segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host cell transformed with a construct which is not normally present in the cell would be considered heterologous for purposes of this invention. Allelic variation or naturally occurring mutational events do not give rise to heterologous DNA, as used herein.

The term “isolated” as also used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

A “packaging cell” refers to a host cell which, by way of stable or transient transfection with heterologous nucleotide sequences, harbors a nucleic acid molecule comprising an viral helper construct, wherein the construct is capable of providing transient expression of packaging functions, e.g., proteins necessary for replication and encapsidation, that can be provided in trans for production of infectious viral particles. Expression of the viral helper functions can be either constitutive, or inducible, such as when the helper functions are under the control of an inducible promoter.

By “recombinant virus” is meant a virus that has been genetically altered, e.g., by the addition or insertion of a heterologous nucleic acid construct into the particle.

The term “OCP (open circle probe)” refers to an oligonucleotide useful in a new method for SNP detection from genomic DNA based on DNA ligase-mediated single nucleotide discrimination and signal amplification by Rolling Circle Amplification (RCA) (see below). In that assay, an oligonucleotide Open Circle Probe (OCP, also called “padlock probe”) anneals to the target SNP such that the 5′ and 3′ ends of the OCP can be ligated together forming a circle topologically linked to the target. A base-pair match between the 3′ end of the OCP and the SNP allows DNA ligase to circularize the OCP. A mismatch between the OCP and the SNP prevents ligation and circularization. In this manner, single base selectivity is achieved not only by the specific hybridization of the OCP ends to target sequences adjacent to the SNP, but also by the highly discriminative nick closure activity of the thermostable DNA ligase toward a perfectly matched substrate. Upon OCP circularization, an isothermal exponential RCA (ERCA) reaction involving an exonuclease(−) DNA polymerase with strand-displacement activity and two primers rapidly amplifies the signal by as much as 10¹²-fold, allowing for direct SNP genotyping from small quantities of DNA target.

As used herein, the term “specifically hybridizes” refers to the ability of a first nucleic acid to hybridize to at least 15 consecutive nucleotides of a second nucleic acid, such as an endogenous gene or gene transcript, such that the hybridization is accompanied by less than 15%, preferably less than 10%, and more preferably less than 5% background hybridization to other cellular or viral nucleic acid (e.g., mRNA or genomic DNA).

As used herein, the terms “transduction” and “transfection” are art recognized and mean the introduction of a nucleic acid, e.g., a viral expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide or, where anti-sense expression occurs from the transferred gene, the expression of a naturally-occurring form of a protein is disrupted.

“Transient transfection” refers to cases where exogenous DNA does not integrate into the genome of a transfected cell, e.g., where episomal DNA is transcribed into mRNA and translated into protein.

A cell has been “stably transfected” with a nucleic acid construct comprising viral coding regions when the nucleic acid construct has been introduced inside the cell membrane and the viral coding regions are capable of being inherited by daughter cells.

As used herein, the term “tissue-specific promoter” means a DNA sequence that serves as a promoter, i.e., regulates expression of a selected DNA sequence operably linked to the promoter, and which effects expression of the selected DNA sequence in specific cells of a tissue, such as cells of neuronal or hematopoietic origin. The term also covers so-called “leaky” promoters, which regulate expression of a selected DNA primarily in one tissue, but can cause at least low level expression in other tissues as well.

“Transcriptional regulatory sequence” is a generic term used throughout the specification to refer to DNA sequences, such as initiation signals, enhancers, and promoters, which induce or control transcription of a gene with which they are operably linked.

As used herein, a “transgenic animal” is any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule.

The “non-human animals” of the invention include vertebrates such as rodents, non-human primates, livestock, avian species, amphibians, reptiles, etc. The term “chimeric animal” is used herein to refer to animals in which the recombinant gene is found.

As used herein, the term “vector” or “plasmid” refers to a nucleic acid molecule capable of transporting another nucleic acid to which it has been linked. One type of vector is an genomic integrated vector, or “integrated vector”, which can become integrated into the chromosomal DNA of the host cell. Another type of vector is an episomal vector, i.e., a nucleic acid capable of extra-chromosomal replication. Vectors capable of directing the expression of genes to which they are operatively linked are referred to herein as “expression vectors”. In the present specification, “plasmid” and “vector” are used interchangeably unless otherwise clear from the context.

Throughout the application, there may be reference to particular transcriptional regulatory sequences, origins of replication, secretion signal sequences, viral vectors, etc. However, it will be appreciated that, unless clearly contrary from the context, many of these specific recitations are intended merely to be illustrative of broader classes of elements which can be used as equivalents.

The term “rolling circle amplification (RCA)” originally refers to a method of replicating a circularized nucleic acid target sequence to generate tandem copies of the target nucleic acid sequence. A single round of amplification using rolling circle amplification results in a large amplification of the circularized probe sequences. However, the same DNA amplification mechanism using the same DNA polymerase can also be applied to linear DNA molecules (For example, see “Hyperbranching RCA,” or “HRCA” below). Thus, RCA as used herein generally refers to DNA amplification using the rolling circle mechanism, which is applicable to both circular and liner nucleic acid molecules.

4.3. Target Nucleic Acid Sequences

In general, the target nucleic acid sequences of the invention may be virtually any sequence of present with a eukaryotic or prokaryotic genomic DNA sequence or other sequence created by genetic engineering such as a vector sequence. Preferred target nucleic acid sequences of the invention are viral expression vectors designed to possess such features as: highly efficient gene transfer; predictable expression levels; coincidence of gene transfer and expression; the ability to identify revertants; relatively easy recovery of the expressed nucleic acid; convenient secondary screens; and facile addition of heterologous DNA, e.g., in library construction. Relative to many other customary mammalian cloning vectors, the subject viral expression vectors also exhibit broad host range specificity for transduction, e.g., so that loss-of-function and/or gain-of-function type constructs can be investigated in biologically relevant cell-types.

In one aspect, the expression cloning systems of the present invention are based on the use of vectors which can be integrated into the genome of a host cell, particularly a mammalian host cell. Exemplary vectors of this sort are derived from e.g., retroviruses, adeno-associated viruses or other virally-derived vectors with appropriate transposition elements for chromosomal integration. Retrovirus vectors and adeno-associated virus vectors are generally understood to be the recombinant gene delivery system of choice for the subject vectors, particularly for use with mammalian cells. These vectors provide efficient delivery of genes into cells, and the transferred nucleic acids are stably integrated into the chromosomal DNA of the host. In addition, the subject vectors also include one or more other features including, for example, a proviral excision element for excising the retroviral vector from the genome of the host cell, a proviral recovery element for enriching and recovering the excised vector, and/or an origin of replication for amplifying or otherwise manipulating the vector in prokaryotic cells. Preferably, the resulting viral vectors are replication-deficient, and, although the virus can have any tropism, they are also preferably amphotrophic with respect to humans. These and other aspects of the subject vectors are described more fully below.

In other embodiments, the expression cloning systems of the present invention are based on episomal vectors which can be maintained at high, but stable, copy numbers in the host cells, and which can deliver uniformly high levels of transcription of a heterologous nucleic acid. In the prior art system, such as the COS cell system discussed above, episomal replication can proceed in a runaway fashion, e.g., resulting in up to 10⁴ episomal copies by 48 hours after transfection. Despite efficient episomal replication in such transient transfectants, low stable transfection efficiencies have been noted (e.g., Chittenden et al, (1991) J Virol 65:5944), presumably because most transfectants die as a result of episome-mediated toxicity. However, the episomal vectors of the present invention provide a strategy for controlling runaway replication to yield episomal copy numbers which can persist through many generations of progeny cells. In preferred embodiments, the episomal vectors of the present invention will include a viral origin of replication, along with other necessary replication control regions, and one or more viral genes that transactivate the viral origin so as to facilitate replication of the vector to a stable copy number. It will be appreciated, however, that the viral transactivating gene(s) can be provided on separate vectors in the cell. Exemplary episomal expression vectors of the present invention include papillomavirus (PV)-derived vectors, Epstein Barr virus (EBV)-derived vectors and BK virus (BKV)-derived vectors.

Expression cloning takes on various forms depending on the mode of detection utilized to identify the nucleic acid of interest (see discussion, infra). However, irrespective of whether the integrating vectors or episomal vectors are utilized, the initial step consists of generating the nucleic acid library, such as by isolating mRNA and synthesizing double-stranded deoxyribonucleic acid copies of the mRNA population (cDNAs). The variegated population of nucleic acids must then be efficiently ligated to a vector of the present invention and transferred to the appropriate host cell prior to library screening and analysis. The subject vectors contain sets of restriction sites, making them amenable to the “adaptor” linker procedure of ligating cDNAs and other nucleic acids into the vector sequences. Also described below are various transcriptional regulatory sequences which can be used to facilitate transcription of the nucleic acid sequence of interest.

A) Retroviral Complementation Screening and Expression Vectors

Retroviruses are RNA viruses; that is, the viral genome is RNA. This genomic RNA is, however, reverse transcribed into a DNA intermediate which is integrated very efficiently into the chromosomal DNA of infected cells. The integrated DNA intermediate is referred to as a provirus. The retroviral genome and the proviral DNA include three genes important to the life cycle of the virus: the gag, the pol and the env genes. The genome of the virus is flanked at each end by long terminal repeat (LTR) sequences. The gag gene encodes the internal structural (nucleocapsid) proteins; the pol gene encodes the RNA-directed DNA polymerase (reverse transcriptase); and the env gene encodes viral envelope glycoproteins. The 5′ and 3′ LTRs serve to promote transcription and polyadenylation of virion RNAs.

Adjacent (downstream) to the 5′ LTR are sequences necessary for reverse transcription of the genome (the tRNA primer binding site) and for efficient encapsidation of viral RNA into particles (the Psi site). Mulligan, R. C., In: Experimental Manipulation of Gene Expression, M. Inouye (ed), 155-173 (1983); Mann et al. (1983) Cell 33:153-159; Cone et al. (1984) PNAS, 81:6349-6353.

If the sequences necessary for encapsidation (or packaging of retroviral RNA into infectious virions) are missing from the viral genome, the result is a cis defect which prevents encapsidation of genomic RNA. However, the resulting mutant, a “replication-deficient” retrovirus, is still capable of directing the synthesis of all virion proteins.

In choosing retroviral vectors, it is also important to note that a prerequisite for the successful infection of target cells by most retroviruses, and therefore of stable introduction of the subject expression constructs, is that the target cells must be dividing. However, while most retroviral vectors require cell division, those based upon lentiviruses, such as HIV or EIAV, do not.

Replication-deficient retroviral vectors compositions are described herein which comprise a combination of features that make possible, for the first time, practical, efficient complementation screening in mammalian cells. Such vectors can also act as efficient expression vectors.

Such retroviral vectors comprise a replication-deficient retroviral genome containing one or more features such as a polycistronic message cassette, a proviral excision element for excising retroviral provirus from the genome of a recipient cell and a proviral recovery element for enriching and recovering excised provirus from a complex mixture of nucleic acid. The vectors are designed to facilitate expression of, for example, cDNA or genomic DNA (gDNA) sequences in mammalian cells.

In an illustrative embodiment, the retroviral vectors may include the following elements: (a) a 5′ retroviral long terminal repeat (5′ LTR); (b) a 3′ retroviral long terminal repeat (3′ LTR); (c) a packaging signal; (d) a bacterial origin of replication; and (e) a bacterial selectable marker. The polycistronic message cassette, proviral recovery element, packaging signal, bacterial origin of replication and bacterial selectable marker are located within the retroviral vector at positions between the 5′ LTR and the 3′ LTR. The proviral excision element, as discussed below, is preferably located within the 3′ LTR. In the alternative, the proviral excision element may also be located within the retroviral vector. However, this is not preferred, since, as elaborated below, one goal of the present invention is to provide a construct wherein the recovered plasmid can be used to directly generate a virus for subsequent rounds of infection.

A variety of different retroviruses are known in the art and can be readily adapted for use in the subject invention. By selection of appropriate amphotropic or ecotropic packaging cell lines, the subject vectors can be packaged as viral particles with suitable specificity for infecting the desired host cell(s). Furthermore, it is also possible to control the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface ligands to the viral env proteins (Neda et al. (1991) J Biol Chem 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to convert an ecotropic vector in to an amphotropic vector, can also be used to limit or expand the specificity of the infectious particle for different cell-types of an animal.

Examples of suitable retroviruses which can be used to generate the subject viral vectors include pBABE, pLJ, pZIP, pWE and pEM, each of which are well known to those skilled in the art. In certain embodiments, the viral vector is derived from a lentivirus, such as a HIV or EIAV virus.

For instance, the pZip vector has been described by Cepko et al. (1984) Cell 37:1053. Briefly, this vector is capable of expressing two genes: the gene of interest and the Neogene as a selectable marker.

The pLJ vector have been described in Korman et al., (1987) PNAS 84:2150. This vector is capable of expressing two genes: the gene of interest and a dominant selectable marker, such as the Neogene. The gene of interest is cloned in direct orientation into a BamHI/Smal/Sall cloning site just distal to the 5′ LTR, while, the Neogene is placed distal to an internal promoter (from SV40) which is farther 3′ than is the cloning site (is located 3′ of the cloning site). Transcription from PLJ is initiated at two sites: 1) the 5′ LTR, which is responsible for expression of the gene of interest and 2) the internal SV40 promoter, which is responsible for expression of the Neogene.

The pWe vector has been described by Choudory et al (1986) CSH Symposia Quantitative Biology 1047. Briefly, this vector can drive expression of two genes: a dominant selectable marker, such as Neo, which is just downstream from the 5′ LTR and a gene of interest which can be cloned into a BamHI site just downstream from an internal promoter capable of high level constitutive expression. Several different internal promoters, such as the beta-actin promoter from chicken (Choudory, P. V. et al, CSH Symposia Quantitative Biology, L.I. 1047 (1986)), and the histone H4 promoter from human (Hanly, S. M. et al., Molecular and Cellular Biology 5:380 (1985)) have been used. Expression of the Neogene is from a transcript initiated at the 5′ LTR; expression of the gene of interest is from a transcript initiated at the internal promoter.

The pEm vector is a simple vector in which the entire coding sequence for gag, pol and env of the wild type virus is replaced with the gene of interest, which is the only gene expressed. The components of the pEm vector are described below. The 5′ flanking sequence, 5′ LTR and 400 bp of contiguous sequence (up to the BAMHI site) is from pZIP. The 3′ flanking sequence and LTR are also from pZIP; however, the Cla site 150 bp upstream from the 3′ LTR has been linked with BamHI and forms the other half of the BamHI cloning site present in the vector. The HindIII/EcoRI fragment of pBR322 forms the plasmid backbone. This vector is derived from sequences cloned from a strain of Moloney Murine Leukemia virus. An analogous vector has been constructed from sequences derived from the myeloproliferative sarcoma virus.

The pIp vector is capable of expressing a single gene driven from an internal promoter. The construction of these vectors is summarized below. The 5′ section of the vector, including the 5′ flanking sequences, 5′ LTR, and 1400 bp of contiguous sequence (up to the XhoI site in the gag region) is derived from wild type Moloney Leukemia virus sequence. Shinnick et al. (1981) Nature 293:543. The difference between the two is that a SacII linker is cloned into an HaeIII restriction site immediately adjacent to the ATG of the gag gene. The 3′ section of the vector, including the 3′ flanking sequences, 3′ LTR and 3′ contiguous sequence (up to the ClaI site in the env coding region) is from pZIP. However, there are two modifications: 1) the ClaI site has been linked to BamHI and 2) a small sequence in the 3′ LTR spanning the enhancer (from PvuII to XbaI) has been deleted. Bridging the 5′ and 3′ sections of the vector is one of several promoters; each one is contained on a XhoI/BamHI fragment, and each is capable of high level constitutive expression in most tissues. These promoters include the β-actin promoter (Choudory et al, supra), and the thymidine kinase promoter from Herpes Simplex Virus (Hanly et al., (1985) Mol Cell Biol 5:380). The vector backbone is the HindIII/EcoRI fragment from pBR322.

The RO vectors represent a heterogeneous group of vectors in which the gene of interest contains all the sequences necessary for transcription (i.e., promoter/enhancer, coding sequence with and without introns, and poly adenylation signal) and is introduced into the retroviral vector in an orientation in which its transcription is in a direction opposite to that of normal retroviral transcription. This makes it possible to include more of the cis-acting elements involved in the regulation of the introduced gene. Virtually, any of the above described genes can be adapted to be a RO vector.

In still other embodiments, it is possible to change the infectivity spectrum of a virus by causing a cell to express a viral receptor (e.g., cell surface protein) which mediates infection by the virus in other species. Thus, for example, human cells can be rendered susceptible to infection with otherwise ecotropic avian virus by causing the human host cells to express an avian gene encoding a receptor for the avian virus.

For embodiments in which it is included, the polycistronic message cassette makes possible a selection scheme which directly links expression of a selectable marker to transcription of a nucleic acid sequence of interest. Such a polycistronic message cassette can comprise, in an exemplary embodiment, from 5′ to 3′, the following elements: a nucleotide polylinker, an (optional) internal ribosome entry site (IRES) and a mammalian selectable marker. The polycistronic cassette is preferably situated within the retroviral vector between the 5′ LTR and the 3′ LTR at a position such that transcription from the 5′ LTR promoter or other transcriptional regulatory sequence transcribes the polycistronic message cassette. In the instance of the latter, the transcription of the polycistronic message cassette may be under the transcriptional control of a constitutive regulatory element, e.g., driven by an internal cytomegalovirus (CMV) promoter, or an inducible regulatory element, as may be preferable depending on the expression screen used. The polycistronic message cassette can further comprise a cDNA, genomic DNA (gDNA) or other nucleic acid sequence operatively associated within the polylinker.

In the subject constructs, the IRES element permits the efficient translation of two or more open reading frames from one messenger RNA: one reading frame, for example, encoding a recombinant protein of interest (such as from a cDNA library) and another an selectable marker (e.g. hygromycin) for selecting cells which express the polycistronic message to some extent.

Bicistronic or multicistronic vectors were developed in order to avoid the problems connected with the stability of the mRNA of different transcripts. For this purpose, the individual reading frames for each transcript (e.g., encoding a protein, providing an antisense transcript, etc.) are provided in a single transcription unit (expression unit). Expression of the multicistronic gene is effected using a single promoter or regulatory sequence. While the first cistron in such vectors is normally translated very efficiently, translation of the subsequent cistrons depends on the intercistronic sequences. It was subsequently possible, with the discovery and use of particular cellular and viral sequences which render possible internal initiation of translation, such as internal ribosome entry sequences or IRES, to achieve a translation ratio between the first and subsequent cistron of 3:1.

A mechanism for initiation translation internally, discovered in recent years, makes use of specific nucleic acid sequences. The sequences include the untranslated regions of individual picorna viruses, e.g. poliovirus and encephalomyocarditis virus, (Pelletier and Sonenberg, (1988) Nature 334:230; Jang et al., (1988) J. Virol. 62:2636; Jang et al., (1989) J. Virol. 63:1641) as well as some cellular proteins, e.g. BiP (Macejak and Sarnow (1991) Nature 353:90-94). In the picorna viruses, a short segment of the 5′ untranslated region, the so-called IRES or internal ribosomal entry site), is responsible for the internal binding of a preinitiation complex. IRES elements can function as initiators of the efficient translation of tandemly linked reading frames. The close coupling of the expression of the selective marker with that of the gene to be expressed is particularly advantageous when selecting for a high level of expression, in particular if prior gene amplification is required.

Internal ribosome entry site sequences are well known to those of skill in the art and can comprise, for example, internal ribosome entry sites derived from foot and mouth disease virus (FDV), encephalomyocarditis virus, poliovirus and RDV (Scheper, 1994, Biochemic 76: 801-809; Meyer, 1995, J. Virol. 69: 2819-2824; Jang, 1988, J. Virol. 62: 2636-2643; Haller, 1992, J. Virol. 66: 5075-5086). Another exemplary bicistronic transcript of the subject vectors contains the 373-nucleotide-long 5′ nontranslated region (NTR) of the classical swine fever virus (CSFV) genome as an intercistronic spacer (Rijnbrand et al. (1997) J Virol 71:451. The ‘R’ regions from HTLV-1 also has properties similar to internal ribosome entry sites (IRES) originally found in picornavirus, Attal et al.(1996) FEBS Lett 392: 220, and can the IRES of that virus can be used in the subject expression constructs. Translation of aphthovirus RNA is initiated at an internal ribosome entry site (IRES) element which can also be used in the subject vectors.

The subject vectors should also include one or more selectable marker genes. Preferably, at least one of the selectable marker genes is provided in a polycistronic transcript with a gene of interest. Any mammalian selectable marker can be utilized. The marker gene is generally one which encodes a product which is necessary for the survival or growth of a host cell transformed with the vector, and/or which can be scored for by a technique which allows cells to be segrated (and retain viability) on the basis of expression of the selectable marker. The expression of this gene product ensures that any host cell which is not transformed with the vector, or which deletes the vector or otherwise losses expression of the selectable marker will not obtain an advantage in growth, etc., over cells retaining a functional vector. Typical selection genes may encode proteins that (a) confer resistance to antibiotics or other toxins, e.g. ampicillin, neomycin, methotrexate or tetracycline, (b) complement auxotrophic deficiencies, or (c) supply critical nutrients not available from complex media.

Examples of suitable drug selectable markers for mammalian cells are dihydrofolate reductase (DHFR), thymidine kinase and genes encoding resistance to kanamycin/G418, hygromycin, mycohenolic acid or neomycin. Such markers enable the identification of cells which were competent to take up, and to retain over time, the subject expression vector. The mammalian cell transformants can be placed under selective conditions wherein only the transformants are uniquely adapted to survive by virtue of having taken up the vector and expressing the marker gene. Selective pressure is imposed, for example, by culturing the transformants under conditions in which the concentration of selection agent in the medium is successively changed, thereby leading to selection of transformant with amplified expression of the selection gene, and, in the polycistronic embodiments, amplified expression of other linked coding sequences.

To illustrate, DHFR⁻ cells which have successfully been transformed with a viral vector including the DHFR selection gene can be identified by culturing the transformants in a culture medium which lacks hypoxanthine, glycine, and thymidine. Cells which can grow under such conditions presumably express the DHFR selection gene provided in the viral vector.

In other embodiments, the marker gene can encode a protein which is detectable by FACS sorting, e.g., the marker gene can be any gene that encodes a FACS detectable gene product, which may be RNA or protein. There are at least two basic designs for such marker genes. In a “direct detection system” the marker gene encodes a product which is readily detectable by flow cytometry due to its own fluorescence activity (a “direct FACS tag”). In the alternative, the marker gene is used in an “indirect detection system”, e.g., wherein the marker gene product is detected by FACS upon combination with a fluorescently active agent which specifically binds to and/or is modified by the marker gene product. Thus, the marker gene may encode a “direct FACS tag”, e.g., a fluorescent polypeptide or a polypeptide which may generate a fluorescent signal by enzymatic action, or an “indirect FACS tag”, e.g., a polypeptide which binds and/or modifies a fluorescently active molecule to generate a fluorescent signal. Chemiluminescent reporter groups, which are for ease of reading referred to herein as fluorescent groups, are detected by allowing them to enter into a reaction, e.g., an enzymatic reaction, that results in energy in the form of light being emitted.

In one embodiment, the marker gene encodes a fluorescently active polypeptide. Examples of such marker genes include, but are not limited to firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); phycobiliproteins (especially phycoerythrin); green fluorescent protein (GFP: see Valdivia et al. (1996) Mol Microbiol 22: 367-78; Cormack et al. (1996) Gene 173 (1 Spec No): 33-8; and Fey et al. (1995) Gene 165:127-130 Both the GFPs and the phycobiliproteins have made an important contribution in FACS sorting generally because of their high extinction coefficient and high quantum yield, and are accordingly preferred products of the marker gene.

A preferred embodiment utilizes a GFP which has been engineered to have a higher quantum yield (brighter) and/or altered excitation spectra relative to wild-type GFPs. In general, the fluorescence levels of intracellular wild-type GFP are not bright enough for flow cytometry. However, a wide variety of engineered GFPs are known in the art which show both improved brightness and signal-to-noise ratios. For instance, the subject reporter gene can encode a GFP-Bex1 (S65T, V163A) or GFP-Vex1 (S202F, T2031, V163A). See Anderson et al. (1996) Genetics 93:8508. Other modified GFPs are described, for example, in U.S. Pat. Nos. 5,360,728 and 5,541,309 which describe modified forms of apoaequorin with increased bioluminescence.

In other embodiments, the marker gene encodes an enzyme which, by acting on a substrate, produces a fluorescently active product. For instance, fluoroscein-di-β-D-galactopyranoside (FDG) is a useful substrate for a marker gene encoding a β-galactosidase in detection by flow cytometry. See Plovins et al. (1994) Applied Envir Micro 60:4638; and Alvarez et al. (1993) Biotechniques 15:974.

In yet other embodiments, the marker gene product is not itself sufficiently fluorescently active for FACS purposes. Rather, the marker gene product is one which is able to bind to a molecule (or complex of molecules), referred to herein as a “secondary fluorescent tag”, which provides a fluorescently active moiety for detection by FACS. A preferred criteria for the selection of the marker gene product in these embodiments is that the host cell, except for the marker gene product, does not produce any other protein, etc., which binds to the secondary fluorescent tag at any appreciable level which would confound the FACS sorting of the host cells.

In preferred embodiments of the indirect detection system, the marker gene encodes a protein which is associated with the cellular membrane and is at least partially exposed to the extracellular milieu. For instance, the indirect FACS tag can be a transmembrane protein having an extracellular domain, or an extracellular protein with some other form of membrane localization signal which keeps the tag sequestered on the surface of the host cell, e.g., such as a myristol, farnesyl or other prenyl group. The indirect FACS tag can be a protein which is native to the host cell, but not normally expressed in the cell either because of its strain or the conditions under which the selection is carried out. In other embodiments, the indirect FACS tag is a protein which includes a portion that is non-native to the host cell, e.g., it is a naturally occurring polypeptide sequence from another species or it is man-made polypeptide sequence, and it is the heterologous portion of the fusion protein which is bound by the secondary fluorescent tag.

Where the marker utilizes an indirect FACS tag, a secondary fluorescent tag must be provided in order to label the cells of FACS. The secondary fluorescent tag can be a fluorescently-labeled antibody or other binding moiety which specifically binds to the indirect FACS tag on the surface of the ITS cell. Where the indirect FACS tag is a receptor, or at least ligand binding domain thereof, the secondary fluorescent tags can also be a fluorescently-labeled ligand of the receptor. Such ligands can be polypeptides or small molecules.

In general, for use in flow cytometry, the fluorescently active tag should preferably have the following characteristics:

(i) the molecules of the secondary fluorescent tag must be of sufficient size and chemical reactivity to be conjugated to a suitable fluorescent dye or the secondary fluorescent tag must itself be fluorescent,

(ii) after any necessary fluorescent labeling, the secondary fluorescent tag preferably does not react with water,

(iii) after any necessary fluorescent labeling, the secondary fluorescent tag preferably does not bind or degrade proteins in a non-specific way, and

(iv) the molecules of the secondary fluorescent tag must be sufficiently large that attaching a suitable dye allows enough unaltered surface area (generally at least 500 Å², excluding the atom that is connected to the linker) for binding to the indirect FACS tag on the cell.

Fluorescent groups with which the process of this invention can be used include fluorescein derivatives (such as fluorescein isothiocyanate), coumarin derivatives (such as aminomethyl coumarin), rhodamine derivatives (such as tetramethyl rhodamine or Texas Red), peridinin chlorophyll complex (such as described in U.S. Pat. No. 4,876,190), and phycobiliproteins (especially phycoerythrin).

In one preferred embodiment of the process, when the marker group is fluorescein, detection of the cells by FACS is achieved by measuring light emitted at wavelengths between about 520 nm and 560 nm (especially at about 520 nm), most preferably where the excitation wavelengths is about or less than 520 nm.

Chemiluminescent groups with which the subject secondary fluorescent tags can be generated include isoluminol (or 4-aminophthalhydrazide).

In other instances, the marker gene can encode a nucleic acid which can be detected by flow cytometry upon interaction with a FACS label. In one embodiment, the marker gene can “encode” a ribozyme, and detection of fluorescently active nucleic acid fragments can be detected for flow sorting upon addition of an appropriately labeled substrate for the ribozyme. For instance, the substrate nucleic acid can include a fluorogenic donor radical, e.g., a fluorescence emitting radical, and an acceptor radical, e.g., an aromatic radical which absorbs the fluorescence energy of the fluorogenic donor radical when the acceptor radical and the fluorogenic donor radical are covalently held in close proximity. See, for example, U.S. Pat. Nos. 5,527,681, 5,506,115, 5,429,766, 5,424,186, and 5,316,691; and Capobianco et al. (1992) Anal Biochem 204:96-102. For example, the substrate nucleic acid has a fluorescence donor group such as 1-aminobenzoic acid (anthranilic acid or ABZ) or aminomethylcoumarin (AMC) located at one position on the polymer and a fluorescence quencher group, such as lucifer yellow, methyl red or nitrobenzo-2-oxo-1,3-diazole (NBD), at a different position. A cleavage site for the ribozyme will be diposed between each of the sites for the donor and acceptor groups. The intramolecular resonance energy transfer from the fluorescence donor molecule to the quencher will quench the fluorescence of the donor molecule when the two are sufficiently proximate in space, e.g., when the substrate is intact. Upon cleavage of the substrate, however, the quencher is separated from the donor group, leaving behind a fluorescent fragment. Thus, expression of the ribozyme results in cleavage of the substrate nucleic acid, and dequenching of the fluorescent group. Similar embodiments can be generated for peptide-based substrates of enzymes.

The retroviral vectors' proviral excision element allows for excision of retroviral provirus (see below) from the genome of a recipient cell. The element comprises a nucleotide sequence which is specifically recognized by a recombinase enzyme, a restriction enzyme, or other enzyme or agent capable of selectively cleaving genomic DNA in a sequence-dependent manner. The recombinase enzyme cleaves nucleic acid at its site of recognition in such a manner that excision via recombinase action leads to circularization of the excised nucleic acid molecules. In the case of restriction enzymes, the excised retroviral sequences can remain linear, or can be circularized by religation.

Enzyme-assisted site-specific integration systems are known in the art and can be applied to the vector system of the invention to excise the viral DNA. Examples of such enzyme-assisted integration systems include the Cre recombinase-lox target system (e.g., as described in Baubonis, W. and Sauer, B. (1993) Nucl. Acids Res. 21:2025-2029; and Fukushige, S. and Sauer, B. (1992) Proc. Natl. Acad. Sci. U.S.A. 89:7905-7909) and the FLP recombinase-FRT target system (e.g., as described in Dang, D. T. and Perrimon, N. (1992) Dev. Genet. 13:367-375; and Fiering, S. et al. (1993) Proc. Natl. Acad. Sci. U.S.A. 90:8469-8473); the Piv site-specific DNA recombinase from Moraxella lacunata (e.g., described by Lenich et al. (1994) J Bacteriol 176) 4160); Lambda integrase (e.g., Kwon et al. (1997) Science 276:126)

By “recombinase target site” (RTS) herein is meant a nucleic acid sequence which is recognized by a recombinase for the excision of the intervening sequence. It is to be understood that two RTSs are required for excision. Thus, when the Cre recombinase is used, each RTS comprises a loxP site; when loxP sites are used, the corresponding recombinase is the Cre recombinase. That is, the recombinase must correspond to or recognize the RTSs. When the FLP recombinase is used, each RTS comprises a FLP recombination target site (FRT); when FRT sites are used, the corresponding recombinase is the FLP recombinase.

A number of different site specific recombinase systems can be used, including but not limited to the Cre/lox system of bacteriophage P1, the FLP/FRT system of yeast, the Gin recombinase of phage Mu, the Pin recombinase of E. coli, and the R/RS system of the pSR1 plasmid. The two preferred site specific recombinase systems are the bacteriophage P1 Cre/lox and the yeast FLP/FRT systems. In these systems a recombinase (Cre or FLP) will interact specifically with its respective site-specific recombination sequence (lox or FRT respectively) to invert or excise the intervening sequences. The sequence for each of these two systems is relatively short (34 bp for lox and 47 bp for FRT). Currently the FLP/FRT system of yeast is the preferred site specific recombinase system since it normally functions in a eukaryotic organism (yeast), and is well characterized.

In a preferred embodiment, the recombinase recognition site is located within the 3′ LTR at a position which is duplicated upon integration of the provirus. This results in a provirus that is flanked by recombinase sites.

In an exemplary embodiment, the proviral excision element comprises a loxP recombination site located in the LTR. Contacting Cre recombinase to an integrated provirus derived from the retroviral vector results in excision of the provirus nucleic acid. In the alternative, a mutant lox P recombination site any be used (e.g., lox P511 (Hoess et al., 1986, Nucleic Acids Research 14:2287-2300)) that can only recombine with an identical mutant site.

In yet another preferred embodiment, an FRT recombination site, which is cleavable by a FLP recombinase enzyme, is utilized in conjunction with FLP recombinase enzyme, as described above for the loxP/Cre embodiment. A “Flip Recombination Target site” (FRT) refers to a nucleotide sequence that serves as a substrate in the site-specific yeast flip recombinase system. The FRT recombination region has been mapped to an approximately 65-base pair (bp) segment within the 599-bp long inverted repeats of the 2-μm circle (a commonly occurring plasmid in Saccharomyces cerevisiae). The enzyme responsible for recombination (FLP) is encoded by the 2-μm circle, and has been expressed at high levels in human cells. FLP catalyzes recombination within the inverted repeats of the molecule to cause intramolecular inversion. FLP can also promote efficient recombination between plasmids containing the 2-μm circle repeat with very high efficiency and specificity. See, e.g., Jayaram (1985) Proc. Natl. Acad. Sci. USA 82:5875-5879; and O' Gorman (1991) Science 251:1351-1355. A “minimum FRT site” (e.g., a minimal FLP substrate) has been described in the art and is defined herein as a 13-bp dyad symmetry plus an 8-bp core located within the 65-bp FRT region. Jayaram et al., supra. Both FRT sites and FLP expression plasmids are commercially available from Stratagene (San Diego, Calif.).

In still another preferred embodiment, an R recombinase site and R recombinase from Zygosaccharomyces rouxii can be utilized, as described above, in place of the loxP/Cre embodiment. EC 2.7.7.-(R recombinase). See also Chen et al. (1991) PNAS 88: 5944.

In yet an alternative embodiment, a rare-cutting restriction enzyme (e.g., Not I) may be used in place of the recombinase site. The recovered DNA would be digested with Not I and then recircularized with ligase. In this embodiment, the Not I site is included in the vector next to loxP. In other embodiments, the restriction enzyme can be 8 or higher base cutter, e.g., requires at least 8 bp for specificity.

In the complementation screening system of the invention, described below, such excision systems can also serve to discriminate revertants from virus-dependent rescue events.

The retroviral vectors' proviral recovery element allows for enrichment or recovery of excised provirus from a complex mixture of nucleic acid, thus allowing for the selective recovery and excision of provirus from a recipient cell genome. The proviral recovery element comprises a nucleic acid sequence which corresponds to the nucleic acid portion of a high affinity binding nucleic acid/protein pair.

The nucleic acid can include, but is not limited to, a nucleic acid which binds with high affinity to a lac repressor, tet repressor or lambda repressor protein. For example, in one embodiment, the proviral recovery element comprises a lac operator nucleic acid sequence, which binds to a lac repressor peptide sequence. Such a proviral recovery element can be affinity-purified using lac repressor bound to a matrix (e.g., magnetic beads or sepharose). An excised provirus derived from the retroviral vectors of the invention also contains the retroviral recovery element and can be affinity purified.

Those skilled in the art will appreciate that there are a wide variety of other DNA binding proteins, including polypeptides derived from naturally occurring DNA binding proteins, as well as polypeptides derived from proteins artificially engineered to interact with specific DNA sequences, which can be used in conjunction with the appropriate proviral recovery element. Basic requirements for the DNA binding protein includes the ability to specifically bind a defined nucleotide sequence.

In one preferred embodiment, the DNA binding protein is derived using all, or a DNA binding portion of a transcriptional regulatory protein, e.g., of either a transcriptional activator or transcriptional repressor, which retains the ability to selectively bind to particular nucleotide sequences. The DNA binding domains of the bacteriophage λcI protein (hereinafter “lambda cI”) and the E. coli LexA repressor (hereinafter “LexA”) represent examples of such DNA binding domains.

However, any other transcriptionally inert or essentially transcriptionally-inert DNA binding domain may be used, such DNA binding domains are well known and include, but are not limited to such motifs as helix-turn-helix motifs (such as found in λcI), winged helix-turn helix motifs (such as found in certain heat shock transcription factors), and/or zinc fingers/zinc clusters. As merely illustrative, the DNA binding protein can be constructed utilizing the DNA binding portions of the LysR family of transcriptional regulators, e.g., Trp1, HvY, OccR, OxyR, CatR, NahR, MetR, CysB, NodD or SyrM (Schell et al. (1993) Annu Rev Microbiol 47:597), or the DNA binding portions of the PhoB/OmpR-related proteins, e.g., PhoB, OmpR, CacC, PhoM, PhoP, ToxR, VirG or SfrA (Makino et al. (1996) J Mol Biol 259:15), or the DNA binding portions of histones H1 or H5 (Suzuki et al. (1995) FEBS Lett 372:215). Other examples include DNA binding portions of the P22 Arc repressor, MetJ, CENP-B, Rap 1, Xy1 S/Ada/AraC, Bir5 or DtxR Furthermore, the DNA binding domain need not be obtained from the protein of a prokaryote. For example, polypeptides with DNA binding activity can be derived from proteins of eukaryotic origin, including from yeast. For example, the DNA binding protein can include polypeptide sequences from such eukaryotic DNA binding proteins as p53, Jun, Fos, GCN4, or GAL4. Likewise, the DNA binding protein can be generated from viral proteins, such as the pappillomavirus E2 protein (c.f., PCT publication WO 96/19566).

In yet other embodiments, the DNA binding protein can be generated by combinatorial mutagenic techniques, and represent a DNA binding domain not naturally occurring in any organism. That is, a completely arbitrary proviral recovery element can be provided in the construct, and such combinatorial approaches used to derive a new protein with sufficient specificity for nucleotide sequence of the element. A variety of techniques have been described in the art for generating novel DNA binding proteins which can selectively bind to a specific DNA sequence (c.f., U.S. Pat. No. 5,198,346 entitled “Generation and selection of novel DNA-binding proteins and polypeptides”).

Thus, the selection of the proviral recovery element is limited only by the availability of a DNA binding protein which recognizes the recovery element's sequence and is compatible with the vector in the host cell and any bacterial cell in which the vector is shuttled/amplified.

In general, the 5′ LTR will include a promoter, including but not limited to an LTR promoter, an R region, a U5 region and a primer binding site, preferably in that order. Nucleotide sequences of these LTR elements are well known to those of skill in the art.

The 3′ LTR comprises a U3 region which comprises the proviral excision element, a promoter, an R region and a polyadenylation signal. Nucleotide sequences of such elements are well known to those of skill in the art.

However, it is also specifically contemplated that the endogenous promoter of the LTR can be replaced with a heterologous transcriptional regulatory sequence, and the 3′ LTR can be replaced with a heterologous polyadenylation signal without effecting the control. For instance, as described in U.S. Pat. No. 5,591,624, the U3 region in a 5′ LTR can be amenable to replacement by a heterologous promoter/enhancer.

The bacterial origin of replication (Ori) utilized is preferably one which does not adversely affect viral production or gene expression in infected cells. As such, it is preferable that the bacterial Ori is a non-pUC bacterial Ori relative (e.g., pUC, colEI, pSC101, p15A and the like). Further, it is preferable that the bacterial Ori exhibit less than 90% overall nucleotide similarity to the pUC bacterial Ori. In a preferred embodiment, the bacterial origin of replication is a RK2 Oriᵥ or f1 phage Ori.

In preferred embodiments, the retroviral vectors can further comprise a single-stranded replication origin, preferably an f1 single-stranded replication origin. The single-stranded replication origin allows for the production of normalized single-stranded retroviral libraries derived from the retroviral vectors of the invention. A normalized library is one constructed in a manner that increases the relative frequency of occurrence of rare clones while decreasing simultaneously the relative frequency of the occurrence of abundant clones. For teaching regarding the production of normalized libraries, see, e.g., Soares et al. (Soares, M. B. et al., 1994, Proc. Natl. Acad. Sci. USA 91:9228-9232, which is incorporated herein by reference in its entirety). Alternative normalization procedures based upon biotinylated nucleotides may also be utilized, and are described in greater detail below.

Any bacterial selectable marker can be utilized. As above, the marker can preferably one which renders the cell resistant to drug treatment, overcomes an auxotrophic phenotype, or provides some other signal which can be directly or indirectly measured and used as a means for selecting bacterial cells which harbor the proviral vector. Bacterial selectable markers are well known to those of skill in the art and can include, but are not limited to, kanamycin/G418, zeocin, actinomycin, ampicillin, gentamycin, tetracycline, chloramphenicol or penicillin resistance markers.

In yet other embodiments, the retroviral vectors can further comprise a lethal stuffer fragment which can be utilized to select for vectors containing cDNA or gDNA inserts during, for example, construction of libraries comprising the retroviral vectors of the invention. Lethal stuffer fragments are well known to those of skill in the art (see, e.g., Bernord et al., 1994, Gene 148:71-74, which is incorporated herein by reference in its entirety). A lethal stuffer fragment contains a gene sequence whose expression conditionally inhibits cellular growth. Thus, by disrupting the expression of the lethal stuffer, e.g., by insertion of the nucleic acid library into the coding sequence of the stuffer fragment, vectors into which the test nucleic acid have been success ligated will no longer express a cytotoxic/cytostatic form of the stuffer fragment. These cells, therefore, can be amplified in the culture by simple virtue of the fact that relief from the inhibitory effects of the stuffer fragments is accorded by the loss-of-function mutation to the stuffer fragment gene by incorporation of the heterologous nucleic acid sequence.

In one embodiment, the stuffer fragment is present in the retroviral vectors of the invention within the polycistronic message cassette polylinker such that insertion of a cDNA or gDNA sequence into the polylinker replaces the stuffer fragment. Alternatively, the polycistronic message cassette polylinker is located within the lethal stuffer fragment coding sequence such that, upon insertion of a cDNA or gDNA sequence into the polylinker, the lethal stuffer fragment coding region is disrupted. Each of these embodiments can be utilized to counter select retroviral vectors not containing polylinker insertions.

B) Adeno-Associated Virus Complementation Screening and Expression Vectors

Yet another viral vector system useful for development of the subject vectors is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J Respir. Cell. Mol. Biol. 7:349-356; Samulski et al. (1989) J. Virol. 63:3822-3828; and McLaughlin et al. (1989) J. Virol. 62:1963-1973). Cis-acting sequences directing viral DNA replication (ori), encapsidation/packaging (pkg) and host cell chromosome integration (int) are contained within the ITRs. Vectors containing as little as 300 base pairs of AAV can be packaged and can integrate. Space for exogenous DNA is limited to about 4.5 kb.

Adeno-associated virus (AAV) is a defective member of the parvovirus family. The AAV genome is encapsidated as a single-stranded DNA molecule of plus or minus polarity (Berns and Rose, 1970, J. Virol. 5:693-699; Blacklow et al., 1967, J. Exp. Med. 115:755-763). Strands of both polarities are packaged, but in separate virus particles (Berns and Adler, 1972, Virology 9:394-396) and both strands are infectious (Samulski et al., 1987, J. Virol. 61:3096-3101).

AAV possesses unique features that make it attractive as a basis for designing the vectors of the present invention. AAV infection of cells in culture is noncytopathic, and natural infection of humans and other animals is silent and asymptomatic. Moreover, AAV infects most (if not all) mammalian cells allowing the possibility of targeting many different tissues in vivo. Kotin et al., (1992) EMBO J. 11:5071-5078 reports that the DNA genome of AAV undergoes targeted integration on chromosome 19 upon infection. Replication of the viral DNA is not required for integration, and thus helper virus is not required for this process. The AAV proviral genome is infectious as cloned DNA in plasmids which makes construction of recombinant genomes feasible. Furthermore, because the signals directing AAV replication, genome encapsidation and integration are contained within the ITRs of the AAV genome, the internal approximately 4.3 kb of the genome (encoding replication and structural capsid proteins, rep-cap) may thus be replaced with foreign DNA such as the gene cassettes described herein, e.g., containing transcriptional regulatory sequences, DNA of interest and a polyadenylation signal. Another significant feature of AAV is that it is an extremely stable and hearty virus. It easily withstands the conditions used to inactivate adenovirus (56 to 65° C. for several hours), making cold preservation of rAAV-based vaccines less critical. Finally, AAV-infected cells are not resistant to superinfection.

The single-stranded DNA genome of the human adeno-associated virus type 2 (AAV2) is 4681 base pairs in length and is flanked by inverted terminal repeated sequences of 145 base pairs each (Lusby et al., 1982, J. Virol. 41:518-526). The first 125 nucleotides form a palindromic sequence that can fold back on itself to form a “T”-shaped hairpin structure and can exist in either of two orientations (flip or flop), leading to the suggestion (B ems and Hauswirth, 1979, Adv. Virus Res. 25:407-449) that AAV may replicate according to a model first proposed by Cavalier-Smith for linear-chromosomal DNA (1974, Nature 250:467-470) in which the terminal hairpin of AAV is used as a primer for the initiation of DNA replication. The AAV sequences that are required in cis for packaging, integration/rescue, and replication of viral DNA appear to be located within a 284 base pair (bp) sequence that includes the terminal repeated sequence (McLaughlin et al., 1988, J. Virol. 62:1963-1973). At least three regions which, when mutated, give rise to phenotypically distinct viruses have been identified in the AAV genome (Hermonat et al., 1984, J. Virol. 51:329-339). The rep region codes for at least four proteins (Mendelson et al., 1986, J. Virol. 60:823-832) that are required for DNA replication and for rescue from the recombinant plasmid. The cap and lip regions appear to encode for AAV capsid proteins; mutants containing lesions within these regions are capable of DNA replication (Hermonat et al., 1984, J. Virol. 51:329-339). AAV contains three transcriptional promoters (Carter et al., 1983, in “The Parvoviruses” K. Berns ed., Plenum Publishing Corp., N.Y. pp. 153-207; Green and Roeder, 1980, Cell 22:231-242; Laughlin et al., 1979, Proc. Natl. Acad. Sci. U.S.A. 76:5567-5571; Lusby and Berns, 1982, J. Virol. 41:518-526; Marcus et al., 1981, Eur. J. Biochem. 121:147-154). The viral DNA sequence displays two major open reading frames, one in the left half and the other in the right half of the conventional AAV map (Srivastava et al., 1985, J. Virol. 45:555-564).

AAV-2 can be propagated as a lytic virus or maintained as a provirus, integrated into host cell DNA (Cukor et al., 1984, in “The Parvoviruses,” Berns ed., Plenum Publishing Corp., N.Y. pp. 33-66). Although under certain conditions AAV can replicate in the absence of helper virus (Yakobson et al., 1987, J. Virol. 61:972-981), efficient replication requires coinfection with either adenovirus (Atchinson et al., 1965, Science 194:754-756; Hoggan, 1965, Fed. Proc. Am. Soc. Exp. Biol. 24:248; Parks et al., 1967, J. Virol. 1:171-180); herpes simplex virus (Buller et al., 1981, J. Virol. 40:241-247) or cytomegalovirus, Epstein-Barr virus, or vaccinia virus. Hence the classification of AAV as a “defective” virus.

When no helper virus is available, AAV can persist in the host cell genomic DNA as an integrated provirus (Berns et al., 1975, Virology 68:556-560; Cheung et al., 1980, J. Virol. 33:739-748). Virus integration appears to have no apparent effect on cell growth or morphology (Handa et al., 1977, Virology 82:84-92; Hoggan et al, 1972, in “Proceedings of the Fourth Lepetit Colloquium,” North Holland Publishing Co., Amsterdam pp. 243-249). Studies of the physical structure of integrated AAV genomes (Cheung et al., 1980, supra; Berns et al., 1982, in “Virus Persistence” Mahy et al., eds., Cambridge University Press, N.Y. pp. 249-265) suggest that viral insertion occurs at random positions in the host chromosome but at a unique position with respect to AAV DNA, occurring within the terminal repeated sequence. Integrated AAV genomes have been found to be essentially stable, persisting in tissue culture for greater than 100 passages (Cheung et al., 1980 supra).

The desirable size of inserted non-AAV or foreign DNA is limited to that which permits packaging of the rAAV vector into virions, and depends on the size of retained AAV sequences. In the generation of the subject constructs, it may be desirable to exclude portions of the AAV genome in the rAAV vector in order to maximize expression of the inserted foreign nucleic acid sequences.

In preferred embodiments, the subject vectors are derived using replication-deficient AAV, e.g., wherein all or a substantial portion of the viral sequence which is naturally flanked by the ITR's is replaced with, for example, a polycistronic expression cassette(s), a bacterial origin of replication, a proviral recovery element, etc., as described for the retroviral vectors described herein. The ITR is also preferably engineered to include a proviral excision element, as described above. All that need be retained are those AAV sequences required for efficient packaging in a helper cell line, along with sequences necessary for chromosomal integration of the viral vector and its stable maintenance.

In this regard, the term “helper virus” refers to a virus, such as adenovirus, herpesvirus, cytomegalovirus, Epstein-Barr virus, or vaccinia virus, which when coinfected with AAV results in productive AAV infection of an appropriate eukaryotic cell. Likewise, helper AAV DNA refers to AAV DNA sequences used to provide AAV functions to a recombinant AAV virus which lacks the functions needed for replication and/or encapsulation of DNA into virus particles. Helper AAV DNA cannot by itself generate infectious virions and may be incorporated within a plasmid, bacteriophage or chromosomal DNA. Finally, helper-free virus stocks of recombinant AAV refers to stocks of recombinant AAV virions which contain no measurable quantities of wild-type AAV or undesirable recombinant AAV.

C. Retrotransposons

Retrotransposons are another example of a class of transposable elements capable of movement from one position to another in the genome. The use of such transposable elements can permit the manipulation of the subject vectors in yeast cells (particularly pathogenic fungus), plant cells, and other eukaryotic cells.

For example, Ty elements of Saccharomyces cerevisiae are retrotransposons that are similar to retroviral proviruses (Bocke, J. D. (1989)) in Mobile DNA, eds., Berg, D. E. & Howe, M. M. (Am. Soc. Microbiol, Washington), pp. 335-374). Retrotransposition is a replicative process involving reverse transcription of Ty mRNA and integration of Ty cDNA into the genome (Boeke et at. Cell (1985) 40:491-500). Ty elements are the most common insertional mutagen and comprise the most numerous family of the four Ty element classes, with about 25-30 copies of Ty1 per haploid genome (Cameron, et al. Cell (1979) 16:739-75 1; Curcio, M. J. et al. Mol. Gen. Genet. (1990) 220:213-221). Despite the fact that Ty1 RNA accounts for 1% of total yeast KNA (Curcio, M. J. et al., supra), the rate of transposition is quite low (Giroux, C. N., et al. Mol. Cell. Biol. (1988) 8:978-981; Boeke, J. D. et al. Mol. Cell. Biol. (1986) 6:3575-3581; Paquin, C. E. et al. Mol. Cell. Biol. (1986) 4:70-79). Several modulators of transposition have been described. For example, Ty transposition is stimulated at temperatures below 30° C. (Paquin, C. E., et al. Science 1984) 226:53-55, by exposure of the cells to ultraviolet irradiation or 4-nitroquinoline 1-oxide (Bradshaw, V. A., et al. Mol. Gen. Genet. (1988) 218:465-474), or in a rad6 mutant background (Picologlou, et al. Mol. Cell. Biol. (1990) 10:1017-1022. Mutations in the SPT3 gene alter the initiation of Ty1 transcription (Winston, et al. Cell (1984) 39:675-682) and abolish retrotransposition of chromosomal Ty1 elements (Boeke, J. D. et al. Mol. Cell. Biol. (1986) 6:3575-3581). These modulators of retrotransposition were identified by their effect on the frequency of Ty insertions into specific loci and not into the genome as a whole. As a result, it can be difficult to determine whether the modulators alter Ty elements directly or the target locus (Picologlou, et al. Mol. Cell. Biol. (1990) 10:1017-1022.

A tremendous induction in the rate of Ty1 transposition is achieved by expressing an active Ty element, Ty1-H3, from the inducible GAL1 promoter (Boeke et al. Cell (1985) 40:491-500). The pGTy1-H3 element has been marked with selectable genes such as a bacterial gene for neomycin resistance (Boeke, et al. Science (1988) 239:280-282) and the yeast HIS3 gene (Garfinkel, et al. Genetics (1988) 120:95-108). Phenotypic detection of retrotransposition events in the transposition-induction system requires loss of the pGTy plasmid. In addition, transposition of the marked Ty1 element can only be detected when it is induced to a level that exceeds the rate of homologous recombination among Ty elements (Roeder, et al. Proc. Natl. Acad. Sci. USA (1982) 79:5621-5625; Roeder, et al. Mol. Cell. Biol. (1984) 4:703-711).

Ty RNA is packaged into virus-like particles (VLPs) that are composed of Ty proteins. Ty elements contain two overlapping genes, TYA and TYB, that are equivalent to retroviral gag and pol. Ty element Gag (TYA)and Gag-Pol (TYA-TYB) polyproteins are cleaved by a Ty-encoded protease (PR) into mature proteins within the VLP. Among these are the mature capsid protein TYA, derived from a precursor protein and PR, integrase (IN), and reverse transcriptase (RT)/RNase H (RH), derived from TYA-TYB polyprotein. (Garfinkel, D. J., The Retroviridae, Plenum Press NY, p. 107-158 (1992).

The retrotransposon utilized in the present vector can be any selected retrotransposon, such as a Saccharomyces cerevisiae Ty element or a Schizosaccharomyces pombe Tf element, the retrotransposon-like element from Aspergillus fumigatus Afut1, the maize Ac/Ds system, copia-like elements of insects such as Drosophilia melanogaster, and VL30 from mice.

A preferable retrotransposon is the budding yeast Ty1 retrotransposon. The term “retrotransposon” as used in the claims includes that substitutions, mutations, additions, etc. can be made to a selected existing retrotransposon. The retrotransposon includes an RT/RH coding sequence, though the functioning of the RT/RH can be altered or destroyed in RT/RH encoded by the vector. Additionally, the retrotransposon preferably includes, e.g., an integrase/transposase coding sequence, a protease coding sequence, terminal repeat sequences, promoter sequence(s) and a sequence encoding a Gag-like protein. Furthermore, the retrotransposon preferably has a Ty protease cleavage site encoded at the natural site of cleavage (between the integrase and the reverse transcriptase) in the polyprotein. This cleavage site preferably provides precise cleavage so that the first amino acid of the RT/RH protein is correct, as exemplified herein.

D. Episomal Complementation and Expression Vectors

As set out above, another aspect of the present invention relates to episomal expression vectors which also can as mammalian expression cloning systems Mammalian episomal vectors, such as the pEHRE vectors described herein, make possible, for the first time, stable, efficient, high-level episomal expression within a wide spectrum of mammalian cells. Such vectors can also, for example, be utilized as part of the complementation screening methods of the invention. The subject episomal vectors are designed to provide high episomal copy numbers, yet not result in runaway replication which could lead to, for example, cell death.

The subject episomal expression vectors, such as the pEHRE vectors, comprise a replication cassette, an expression cassette and minimal cis-acting elements necessary for replication and stable episomal maintenance.

The episomal vectors of the invention can further contain at least one bacterial origin of replication and/or recombination sites. The recombination sites preferably flank the replication cassette, and can include, but are not limited to, any of the recombination sites described above.

Any bacterial origin of replication (Ori) which does not adversely affect the expression of the coding sequences provided in the expression vector can be utilized. For example, the bacterial Ori can be a pUC bacterial Ori relative (e.g., pUC, colEI, pSC101, p15A and the like). The bacterial origin of replication can also, for example, be a RK2 Oriv or f1 phage Ori. The pEHRE vectors can further comprise a single stranded replication origin, preferably an f1 single-stranded replication origin. The single-stranded replication origin allows for the production of normalized single-stranded libraries derived from the pEHRE vectors of the invention. A normalized library is one constructed in a manner that increases the relative frequency of occurrence of rare clones while decreasing simultaneously the relative frequency of the occurrence of abundant clones. For teaching regarding the production of normalized libraries, see, e.g., Soares et al. (Soares, M. B. et al., 1994, Proc. Natl. Acad. Sci. USA 91:9228-9232, which is incorporated herein by reference in its entirety). Alternative normalization procedures based upon biotinylated nucleotides may also be utilized.

In instances wherein an f1 origin of replication is utilized, the pEHRE vectors can additionally comprise a nucleic acid sequence which corresponds to the nucleic acid portion of a high affinity binding nucleic acid/protein pair. Such nucleic acid/protein pairs can be as described above, the nucleic acid portion of which can include, but is not limited to, a lacO site. The nucleic acid can include, but is not limited to, a nucleic acid which binds with high affinity to a lac repressor, tet repressor or lambda repressor protein. For example, in one embodiment, the proviral recovery element comprises a lac operator nucleic acid sequence, which binds to a lac repressor peptide sequence. Such a proviral recovery element can be affinity-purified using lac repressor bound to a matrix (e.g., magnetic beads or sepharose). An excised provirus derived from the retroviral vectors of the invention also contains the retroviral recovery element and can be affinity purified.

In an exemplary embodiment, a pEHRE vector replication cassette comprises nucleic acid sequences which encode papillomaviruses (PV) E1 and E2 proteins, wherein such nucleic acid sequences are operatively attached to and transcribed by, a constitutive or inducible transcriptional regulatory sequence, though constitutive is preferred. Representative E1 and E2 amino acid sequences are well known to those of skill in the art. See, e.g., sequences publicly available in databases such as Genbank. The E1 and E2 coding sequences can, first, include any nucleotide sequences which encode endogenous PV, including but not limited to bovine papillomavirus (BPV), such as BPV-1 E1 or E2 gene products.

As used herein, the term “E1” also refers to any protein which is capable of functioning in PV in the same manner as the endogenous E1 protein, i.e., is capable of complementing an E1 mutation. Taking BPV as an example, an E1 protein, as described herein, is one capable of complementing a BPV E1 mutation. Likewise, the term “E2”, as used herein, refers to any protein which is capable of functioning in PV in the same manner as the endogenous E2 protein, i.e., is capable of complementing a E2 mutation. Taking BPV as an example, an E2 protein, as described herein, is one capable of complementing a BPV E2 mutation.

The replication cassette transcriptional regulatory sequence can include, but is not limited to, any polII promoter, such as an SV40, CMV or PGK promoter, nucleotide sequences of which are well known to those of skill in the art.

E1 and E2 coding sequences can be operatively attached to, and transcribed by, separate transcriptional regulatory sequences. However, it is preferred that at least one, and more preferably both of the E1 and E2 sequences are provided in polycistronic arrangements, alone or together, with at least one selectable marker (discussed infra). In one embodiment, at least one of the E1 or E2 coding sequences can be transcribed along with a selectable marker as a polycistronic message. Such a polycistronic message construction makes possible a selection scheme which directly links expression of a selectable marker, preferably a mammalian selectable marker, to transcription of a sequence necessary for episomal maintenance and replication. For example, the portion of a replication cassette encoding such a polycistronic message could comprise, from 5′ to 3′: a constitutive transcriptional regulatory sequence, an E2 (or E1) coding sequence, an internal ribosome entry site (IRES), and a selectable marker.

In another embodiment, both E1 and E2 coding sequences can be transcribed together as part of a polycistronic message. That is, both E1 and E2 coding sequences, separated by an internal ribosome entry site, can be transcribed by a single transcriptional regulatory sequence.

In yet another embodiment, E1, E2 and selectable marker sequences can be transcribed as a polycistronic message. For example, the replication cassette could comprise, from 5′ to 3′ : a constitutive transcriptional regulatory sequence, an E2 (or E1) coding sequence, an IRES, an E1 (or E2) coding sequence, an IRES and a selectable marker.

In instances wherein the E1 and E2 coding sequences are transcribed as part of a polycistronic message, it is preferred that the order, from 5′ to 3′, be E2 then E1. This is to ensure against possible rare, undesirable RNA splicing events.

The episomal expression constructs of the present invention are derived to yield high level expression of a cDNA, genomic DNA (gDNA) or other nucleic acid sequence. Such a pEHRE vector expression cassette comprises, from 5′ to 3′, a transcriptional regulatory sequence, a nucleotide polylinker, an internal ribosome entry site, a mammalian selectable marker and, preferably, either a poly-A site or a transcriptional termination sequence, depending upon the transcriptional regulatory sequence utilized (see below). A cDNA or gDNA sequence can be expressed via operative association within the polylinker. A pEHRE expression vector can contain a single or multiple expression cassettes, such that greater than one cDNA or gDNA sequence can be expressed from the same pEHRE expression vector.

The pEHRE vector expression cassette transcriptional regulatory sequence can be either constitutive or inducible, and can be derived from cellular or viral sources. For example, such transcriptional regulatory sequences can include, but are not limited to, a retroviral long terminal repeat (LTR), cytomegalovirus (CMV), Va-1 RNA or U6 snRNA promoter sequence, nucleotide sequences of which are well known to those of skill in the art. Depending upon the transcriptional regulatory sequence chosen, the expression cassette can contain either a poly-A site (pA) or a transcriptional termination sequence. One of skill in the art will readily be able to choose, without undue experimentation, the appropriate sequence to be used with any given transcriptional regulatory sequence. In general, for example, polII-type transcriptional regulatory sequences can be coupled with pA sites, and polIII-type transcriptional regulatory sequences can be coupled with transcriptional termination sequences.

Expression from the transcriptional regulatory sequence yields a polycistronic message comprising the cDNA or gDNA sequence of interest, IRES and mammalian selectable marker. Such a polycistronic message approach allows a selection scheme which ensure that the cDNA or gDNA of interest has been expressed.

The pEHRE vectors further comprise cis-acting elements which function in replication and stable episomal maintenance. Such sequences include: a PV minimal origin of replication (MO) and a PV minichromosomal maintenance element (MME). Representative MO and MME sequences are well known to those of skill in the art. See, e.g., Piirson, M. et al., 1996, EMBO J. 15:1 -11, which is incorporated herein by reference in its entirety.

As used herein, the term “MO” refers to any nucleotide sequence capable of functioning in PV in the same manner as endogenous MO, i.e., is capable of complementing an MO mutation. Taking BPV as an example, an MO sequence, as described herein, would be one capable of complementing or replacing a BPV MO mutation. Likewise, the term “MME”, as used herein, refers to any nucleotide sequence capable of functioning in PV in the same manner as endogenous MME, i.e., is capable of complementing a MME mutation. For example, a MME sequence can be one containing multiple E2 binding sites. Taking BPV as an example, a MME sequence, as described herein, would be one capable of complementing or replacing a BPV MME mutation.

The pEHRE IRES and mammalian and bacterial selectable markers can be, for example, as those described above.

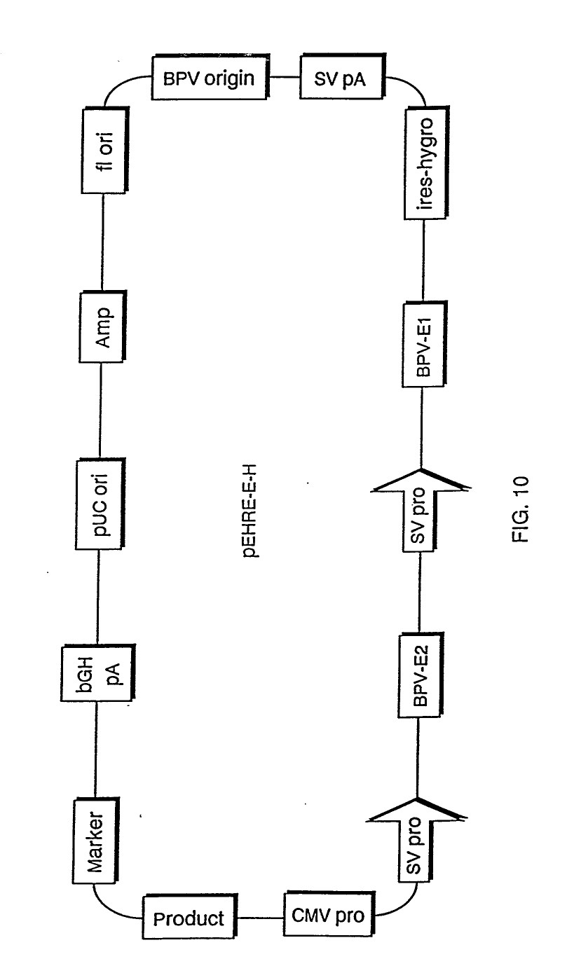

Depicted in FIG. 10 is an example of one pEHRE vector embodiment, termed pEHRE-E-H. In this vector, the E1 and E2 coding sequences are BPV sequences, and are in operative association with individual SV40 promoters. E1 is transcribed as part of a polycistronic message along with the selectable marker, hygro. In this embodiment, the replication cassette further comprises an SV40 pA site downstream of the IRES-marker. Further, the MO and MME sequences are BPV-derived (in the figure, both of these sequences are illustrated as “BPV origin”). The vector's expression cassette comprises a CMV promoter operatively associated with a sequence to be expressed (“product”), said sequence in operative association with an IRES-marker (the sequence to be expressed and the IRES-marker are illustrated as “marker” in the figure), which, in turn, is in operative association with a bgH poly-A site. Finally, the vector contains a pUC bacterial origin (Ori) of replication, an f1 Ori and an ampicillin bacterial selectable marker.

The episomal expression vectors of the invention, such as pEHRE, can be utilized for the production, including large scale production, of recombinant proteins. The vectors' desirable features, in fact, make them especially amenable to large scale production. Specifically, current methods of producing recombinant proteins in mammalian cells involve transfection of cells (e.g., CHO, NS/0 cells) and subsequent amplification of the transfected sequence using drugs (e.g., methotrexate or inhibitors of glutamine synthetase). Such approaches suffer for a variety of reasons, including the fact that amplicons are subject to statistical variation depending on their genomic integration loci, and from the fact that the amplicons are unstable in the absence of continued selection (which is impractical at production scale). The subject vectors, it should be pointed out, achieve such levels equal or higher than these naturally, that is, in the absence of outside selection.

Thus, the present invention provides a means for producing such proteins as proteins such as human serum albumin; human interferons; human antibodies; human insulin; erythropoietin, steel factor and other hematopoietic factors; blood clotting factors, particularly the rare human blood clotting factors such as Factor IX or VIII; thrombolytic factors such as tissue plasminogen activators; human growth factors; brain peptides; interleukins; endorphins; enzymes; prolactin; viral antigens; and even plant proteins.

The pEHRE vectors of the invention, in contrast, give consistently high episomal expression, making them genomic integration-independent. Further, the episomal pEHRE vectors are retained as stable nuclear plasmids even in the absence of selective pressure.

Further, pEHRE vectors can be utilized which employ an additional level of such internal, or self, selection (that is, selection which does not depend on the addition of outside selective pressures such as, e.g., drugs). For example, pEHRE vectors can be utilized which complement a defect the specific producer cell line being utilized for expression. By way of example, and not by way of limitation, such pEHRE selection elements can complement an auxotrophic mutation or can bypass a growth factor requirement (e.g., proline or insulin, respectively) from the cell media. Preferably, the coding sequence of the marker is transcribed as part of a polycistronic message along with the coding sequence of the proteins being recombinantly expressed. For example, such an expression/selection cassette can comprise, from 5′ to 3′: a transcriptional regulatory sequence, recombinant protein coding sequence, IRES, selection marker, poly-A site.

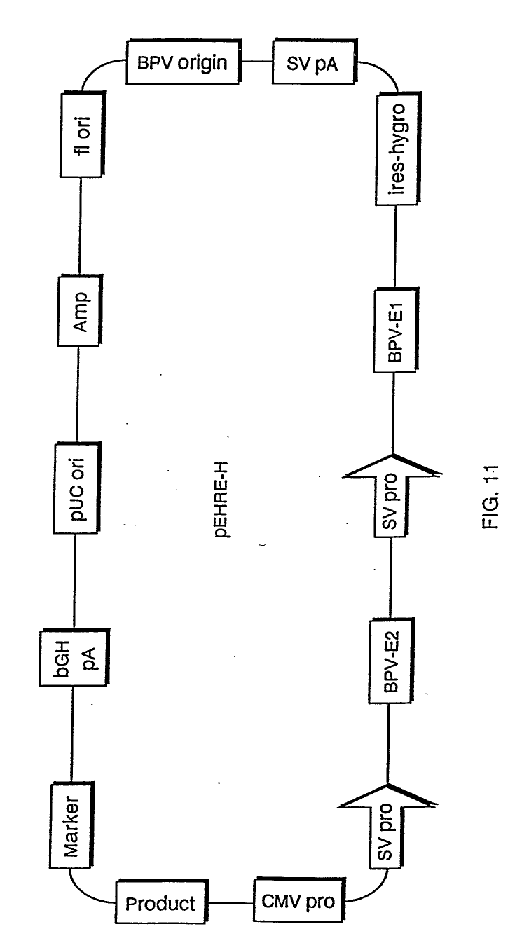

The vector depicted in FIG. 11, termed pEHRE-H, depicts one embodiment of a pEHRE vector that can be utilized for large scale production. The “Marker” element represents a “self-selection” marker as discussed above operatively attached to an IRES. “Product” in the figure refers to the coding sequence of the recombinant protein being expressed. The remainder of the elements of the vector are as described for the vector presented in FIG. 10, above.

The episomal pEHRE vectors of the invention can further be utilized, for example, in the delivery of large nucleic acid segments, e.g., chromosomal segments. In one such embodiment, pEHRE vectors can be utilized in connection with bacterial artificial chromosome (BAC) or yeast artificial chromosome (YAC) sequences to allow delivery of large genomic segments (e.g., segments ranging from tens of kilobases to megabases in length). For clarity, the discussion that follows describes vectors that utilize BAC sequences, but it is to be understood that vectors of the sort described here can, alternatively, utilize YAC sequences.

In one embodiment, pEHRE vectors can be combined with existing BAC clones to generate pEHRE/BAC hybrid constructs, comprising BACs into which pEHRE vector sequences have been inserted. Such pEHRE/BAC hybrids represent BACs that can replicate in a wide variety of mammalian, including human cells.

In general, pEHRE vectors which can be utilized to donate elements to BACs comprise a pEHRE replication cassette, MO and MME sequences, and a bacterial selectable marker, all flanked by BAC recombination sequences. The remainder of the vector can further comprise at least one bacterial origin of replication and a second bacterial selectable marker.

BAC recombination sequences can include any nucleotide sequence which can be cleaved and then used to recombine with BAC elements so as to incorporate the necessary pEHRE sequences described above. Any recombination site for which a compatible recombination site exists, or is engineered to exist, in the recipient BAC can be used. For example, such BAC recombination elements can include, but are not limited to, loxP, mutant loxP or FRT sites as described above.

Alternatively, CosN sites, whose nucleotide sequences are well known to those of skill in the art, can be utilized. Rather than a recombinase enzyme, such CosN sites are cleaved by lambda terminase enzyme. (For general BAC teaching, including CosN teaching, see, e.g., Shizuya, H. et al., 1992, Proc. Natl. Acad. Sci. USA 89:8794-8797; and Kim, U.-J. et al., 1996, Genomics 34:213-218, which are incorporated herein by reference in their entirety.)

In order to recombine pEHRE and BAC sequences, pEHRE vectors and BAC (containing a recombination site compatible with the chosen pEHRE vector) are treated together with the appropriate recombinase or terminase enzyme. When the CosN/terminase system is used, a subsequent ligation step is included.

The treatment will result in a low level of concatamerization. Concatamers representing the desired pEHRE/BAC hybrids can be selected for based upon their resistance to both the BAC selectable marker (usually chloramphenicol) and the pEHRE vector selectable marker within the pEHRE region meant to be donated. It is, therefore, desirable that the BAC and pEHRE selectable markers be different. In a preferred embodiment, the resulting constructs are further tested to ensure that the second pEHRE bacterial selectable marker is no longer present. Plasmids which have recombined the desired BAC and pEHRE elements, will be able to replicate in E. coli, as well as a wide range of mammalian cells, including human cells.

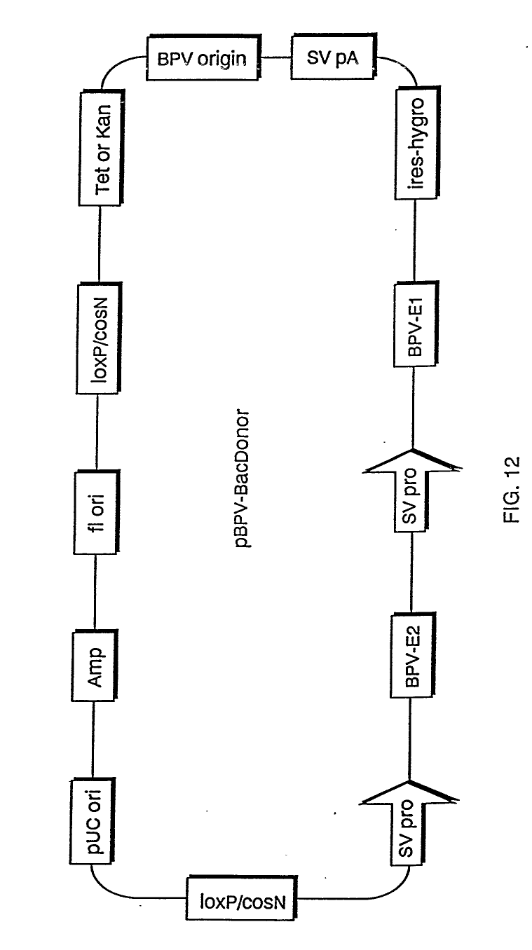

The vector depicted in FIG. 12, termed a pBPV-BacDonor vector, represents one embodiment of a pEHRE vector designed to donate essential pEHRE sequences to recipient BAC clones. The vector's recombination elements are depicted as containing loxP and/or CosN sites. The bacterial marker to be incorporated into the pEHRE/BAC hybrid is depicted as tetracycline or kanamycin. Finally, the vector contains a pUC bacterial origin (Ori) of replication, an f1 Ori and a second bacterial selectable marker, ampicillin.

In an alternative embodiment, pEHRE/BAC cloning vectors can be produced and utilized. Such vectors contain the pEHRE replication cassette, MO and MME sequences as described above, the nucleotide sequences necessary for BAC maintenance in E. coli (such sequences are well known to those of skill in the art; see, e.g., Shizuya and Kim, above), and a polylinker site.

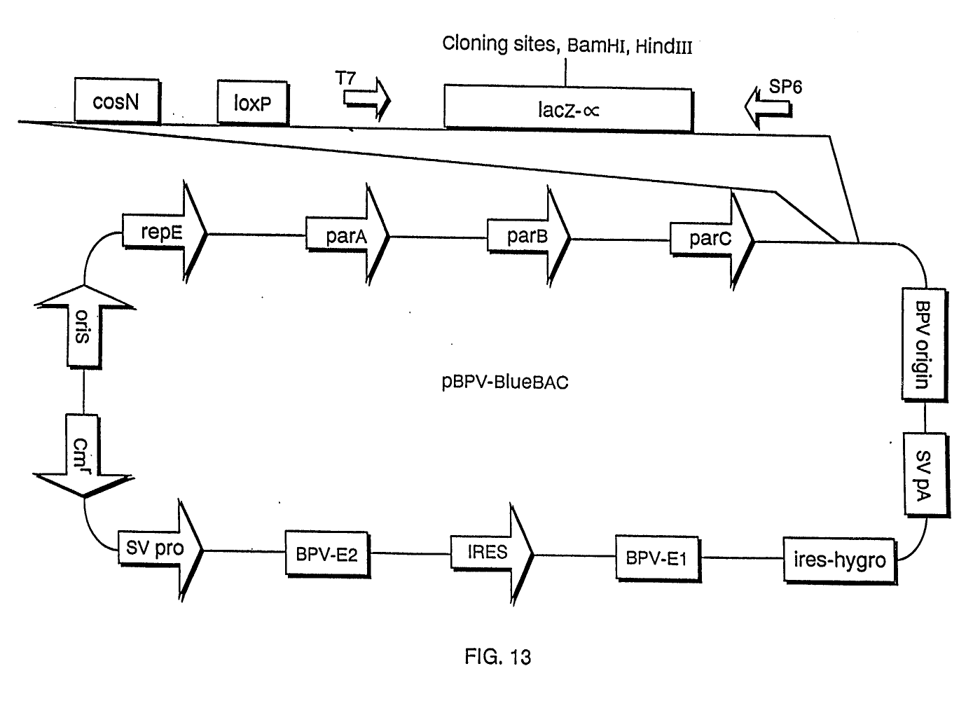

The vector depicted in FIG. 13, termed pBPV-BlueBAC, represents one embodiment of such a pEHRE/BAC cloning vector. In this vector, the E1 and E2 coding sequences are BPV sequences, and are in operative association with individual SV40 promoters. E1 is transcribed as part of a polycistronic message along with the selectable marker, hygro. In this embodiment, the replication cassette further comprises an SV40 pA site downstream of the IRES-marker. Further, the MO and MME sequences are BPV-derived (in the figure, both of these sequences are illustrated as “BPV origin”). The cloning site comprises a polylinker embedded within the alpha complementation fragment of lacZ, which allows blue/white selection of recombinants. T7 and SP6 promoters flank the lacZ sequence, and the vector additionally contains cosN and loxP sites for linearization. The remainder of the elements depicted are present for BAC maintenance in E. coli.

E) Antisense-gse Retroviral Vectors

Described herein are genetic suppressor element (GSE)-producing, replication-deficient retroviral vectors. Such vectors are designed to facilitate the expression of antisense GSE single-stranded nucleic acid sequences in mammalian cells, and can, for example, be utilized in conjunction with the antisense-based functional gene inactivation methods of the invention. The GSE element can also be a ribozyme, e.g., a hammerhead ribozyme or the like, which is being designed to, for example, inhibit expression of a target gene.

The GSE-producing retroviral vectors of the invention can comprise a replication-deficient retroviral genome containing a proviral excision element, a proviral recovery element and a genetic suppressor element (GSE) cassette.

The GSE-producing retroviral vectors can further comprise, (a) a 5′ LTR; (b) a 3′ LTR; (c) a bacterial Ori; (d) a mammalian selectable marker; (e) a bacterial selectable marker; and (f) a packaging signal.

The proviral recovery element, GSE cassette, bacterial Ori, mammalian selectable marker and bacterial selectable marker are located between the 5′ LTR and the 3′ LTR. The proviral excision element is located within the 3′ LTR. The proviral excision element can also flank the functional cassette without being present in the 3′ LTR.

The 5′ LTR, 3′ LTR, proviral excision element, bacterial selectable marker, mammalian selectable marker and proviral recovery element are as described above.

Each of the GSE cassette embodiments described below can further comprise a sense or antisense cDNA or gDNA fragment or full length sequence operatively associated within the polylinker. Moreover, the GSE cassettes can be oriented to transcribe in either the same or opposite orientation with respect to the LTR driving its transcription. That LTR can also be an intact LTR, or a self-inactivating (SIN) LTR.

The GSE cassette can, for example, comprise, from 5′ to 3′: (a) a transcriptional regulatory sequence; (b) a polylinker; and (c) polyadenylation signal. In one embodiment, the GSE cassette polyadenylation signal is located within the 3′ retroviral long terminal repeat.

Alternatively, the GSE cassette can comprise, from 5′ to 3′: (a) a transcriptional regulatory sequence; (b) a polylinker; (c) a cis-acting ribozyme sequence; (d) an internal ribosome entry site; (e) the mammalian selectable marker; and (f) a polyadenylation signal.

In a further alternative, a sense GSE can be constructed, in which case the GSE cassette can further comprise a polylinker containing a Kozak consensus methionine in front of the sense-orientation fragments to create a “domain library” for domain and fragment expression.

In such an embodiment, transcription from the transcriptional regulatory sequence produces a bifunctional transcript. The first half (i.e., the portion upstream of the ribozyme sequence) is likely to remain nuclear and represents the GSE. The portion downstream of the ribozyme sequence (i.e., the portion containing the selectable marker) is transported to the cytoplasm and translated. Such a bicistronic configuration, therefore, directly links selection for the selectable marker to expression of the GSE.

In another alternative, the GSE cassette can comprise, from 5′ to 3′: (a) an RNA polymerase III transcriptional regulatory sequence; (b) a polylinker; (c) a transcriptional termination sequence. In a particular embodiment, the transcriptional regulatory sequence and transcriptional termination sequence are adenovirus Ad2 VA RNAI transcriptional regulatory and termination sequences.

F) pEHRE Antisense-Genetic Suppressor Element Vectors

Described herein are genetic suppressor element (GSE)-producing, pEHRE vectors. Such vectors are designed to facilitate the expression of antisense GSE single-stranded nucleic acid sequences in mammalian cells, and can, for example, be utilized in conjunction with the antisense-based functional gene inactivation methods of the invention.

The GSE-producing pEHRE vectors of the invention can comprise a replication cassette, a genetic suppressor element (GSE) cassette and minimal cis-acting elements necessary for replication and stable episomal maintenance.

The GSE-producing pEHRE vectors can further comprise at least one bacterial origin of replication and at least one bacterial selectable marker.

The replication cassette, minimal cis-acting elements, bacterial origin of replication and bacterial selectable marker are as described above.

Each of the GSE cassette embodiments described below can further comprise a sense or antisense cDNA or gDNA fragment or full length sequence operatively associated within the polylinker.

The GSE cassette can, for example, comprise, from 5′ to 3′: (a) a transcriptional regulatory sequence; (b) a polylinker; and (c) polyadenylation signal. The GSE transcriptional regulatory sequence can be a constitutive or inducible one, and can represent, for example, retroviral long terminal repeat (LTR), cytomegalovirus (CMV), Va-1 RNA or U6 snRNA promoter sequence, nucleotide sequences of which are well known to those of skill in the art.

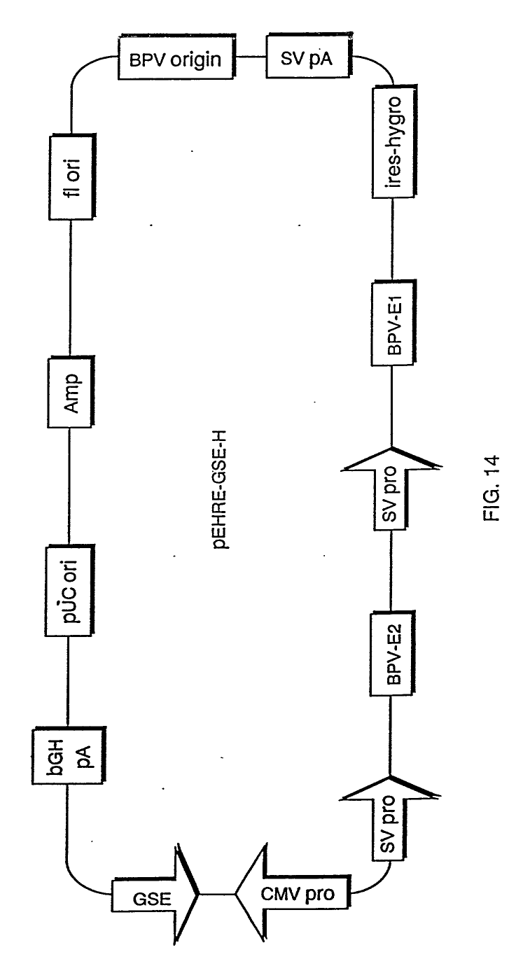

The vector depicted in FIG. 14 represents an example of such a pEHRE GSE vector. In this vector, the E1 and E2 coding sequences are BPV sequences, and are in operative association with individual SV40 promoters. E1 is transcribed as part of a polycistronic message along with the selectable marker, hygro. In this embodiment, the replication cassette further comprises an SV40 pA site downstream of the IRES-marker. Further, the MO and MME sequences are BPV-derived (in the figure, both of these sequences are illustrated as “BPV origin”). The vector's GSE cassette comprises a CMV promoter operatively associated with a sequence to be expressed as a GSE, which, in turn, is operatively attached to a bgH poly-A site. Finally, the vector contains a pUC bacterial origin (Ori) of replication, an f1 Ori and an ampicillin bacterial selectable marker.

Alternatively, the GSE cassette can comprise, from 5′ to 3′: (a) a transcriptional regulatory sequence; (b) a polylinker; (c) a cis-acting ribozyme sequence; (d) an internal ribosome entry site; (e) the mammalian selectable marker; and (f) a polyadenylation signal.

In another alternative, a sense GSE can be constructed, in which case the GSE cassette can further comprise a polylinker containing a Kozak consensus methionine in front of the sense-orientation fragments to create a “domain library” for domain and fragment expression.

In such an embodiment, transcription from the transcriptional regulatory sequence produces a bifunctional transcript. The first half (i.e., the portion upstream of the ribozyme sequence) is likely to remain nuclear and represents the GSE. The portion downstream of the ribozyme sequence (i.e., the portion containing the selectable marker) is transported to the cytoplasm and translated. Such a bicistronic configuration, therefore, directly links selection for the selectable marker to expression of the GSE.

In another alternative, the GSE cassette can comprise, from 5′ to 3′: (a) an RNA polymerase III transcriptional regulatory sequence; (b) a polylinker; (c) a transcriptional termination sequence.

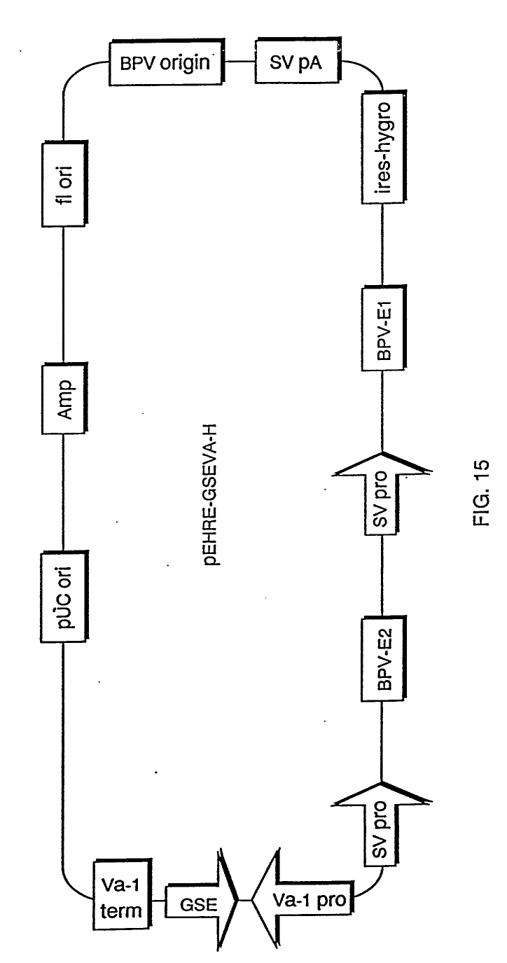

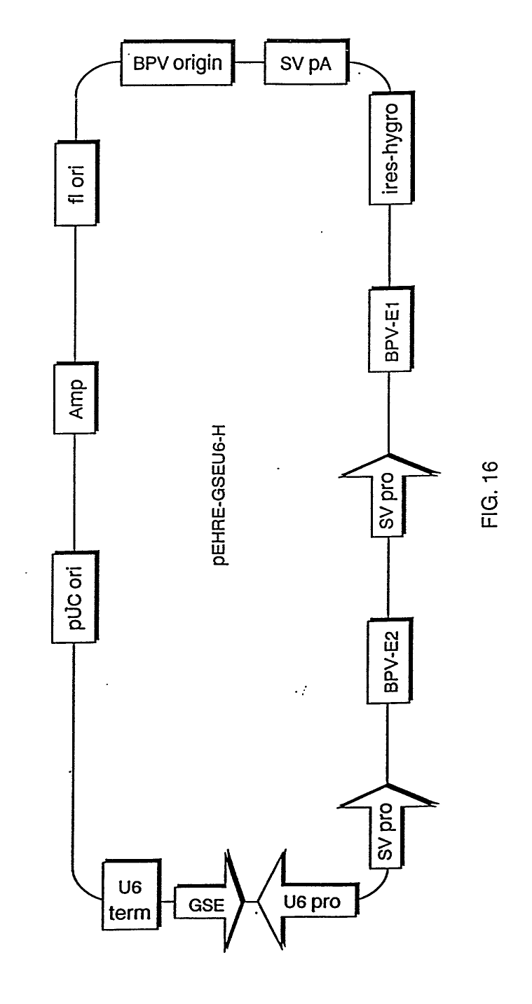

The vectors depicted in FIGS. 15 and 16 represent examples of this type of pEHRE GSE vector. The GSE cassette of the vector depicted in FIG. 15 comprises a Va-1 promoter which is operatively attached to a sequence to be expressed as a GSE, which is, in turn, operatively attached to a Va-1 termination sequence. The GSE cassette of the vector depicted in FIG. 16 comprises a U6 promoter which is operatively attached to a sequence to be expressed as a GSE, which is, in turn, operatively attached to a U6 termination sequence. The remainder of the elements depicted in the FIGS. 15 and 16 vectors are as described for the vector shown in FIG. 14.

In a particular embodiment, the transcriptional regulatory sequence and transcriptional termination sequence are adenovirus Ad2 VA RNA transcriptional regulatory and termination sequences.

G) Linked Marker for Antisense Development

An important use for antisense libraries comes in the refinement/optimization of the antisense sequences which can be used to effectively inhibit expression of a gene, or function of a structural RNA element. In order to provide high through screening techniques for detecting effective antisense sequences, the subject invention also provides a linked marker construct providing a convenient readout on the level of expression of the targeted gene. In particular, the linked marker is a fusion gene comprised of a coding or non-coding sequence for which an antisense construct is sought, e.g., it can include the coding sequence for a target protein. The fusion gene also includes a coding sequence for a marker protein, e.g., a protein whose expression can be detected, and preferably quantitated. A variety of marker genes are described above for selection, and many of those can be used to generate the subject linked marker. For instance, the marker can be a cell surface marker, a detectable enzyme, a gene product which complements a condition of the host cell, a transcription factor, etc. In preferred embodiments, the target sequence and linked marker encode a fusion protein including the marker protein. In the absence of antisense effective for inhibiting the expression of the target protein, the linked marker will be expressed and detected. However, antisense which can inhibit the expression of the target protein, e.g., by hybridizing to the fusion gene or a transcript thereof, will cause a reduction in the level of detectable marker. This method can also be used to screen libraries of ribozymes, e.g., hammerhead ribozymes, in order to identify ribozymes able to inhibit expression of the target gene.

According to one aspect of the invention, there is provided a library of vectors of the present invention including variegated population of transcrible gene sequences which, upon transcription, provide a population of potential antisense transcripts for a gene, e.g., a mammalian gene.

After identification in the subject method, one or more of the antisense sequences identified can be provided in a pharmaceutical preparation suitable for antisense therapy. As used herein, “antisense” therapy refers to administration or in situ generation of oligonucleotide probes or their derivatives which specifically hybridize (e.g. bind) under cellular conditions with cellular mRNA and/or genomic DNA encoding a target protein. The hybridization should inhibit expression of that protein, e.g. by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct identified by the method of the present invention can be prepared for in vivo delivery, for example, as an expression plasmid which, when transcribed in the cell, produces RNA which is complementary to at least a unique portion of the target cellular mRNA. Alternatively, the antisense construct is an oligonucleotide probe which is generated ex vivo and which, when introduced into the cell causes inhibition of expression by hybridizing with the mRNA and/or genomic sequences of a target gene. Such oligonucleotide probes are preferably modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668.

H) Vectors Displaying Random Peptide Sequences

Described herein are vectors usefull for the display of constrained and unconstrained random peptide sequences. Such vectors are designed to facilitate the selection and identification of random peptide sequences that bind to a protein of interest.

The integrated and episomal vectors of the present invention can be engineered to display random peptide sequences. Such vectors of the present invention can comprise, to illustrate, (a) a splice donor site or a LoxP site (e.g., LoxP511 site); (b) a bacterial promoter (e.g., pTac) and a shine-delgarno sequence; (c) a pel B or other secretion signal sequence for targeting fusion peptides to the periplasm; (d) a splice-acceptor site or another LoxP511 site (Lox P511 sites will recombine with each other, but not with the LoxP site in the 3′ LTR); (e) a peptide display cassette or vehicle; (f) an amber stop codon; (g) the M13 bacteriophage gene 111 protein C-terminus (amino acids 198-406); and optionally the vector may also comprise a flexible polyglycine linker.

A peptide display cassette or vehicle consists of a vector protein, either natural or synthetic into which a polylinker has been inserted into one flexible loop of the natural or synthetic protein. A library of random oligonucleotides encoding random peptides may be inserted into the polylinker, so that the peptides are expressed on the cell surface.

The display vehicle of the vector may be, but is not limited to, thioredoxin for intracellular peptide display in mammalian cells (Colas et al., 1996, Nature 380:548-550) or may be a minibody (Tramonteno, 1994, J. Mol. Recognit. 7:9-24) for the display of peptides on the mammalian cell surface. Each of these would contain a polylinker for the insertion of a library of random oligonucleotides encoding random peptides at the positions specified above. In an alternative embodiment, the display vehicle may be extracellular, in this case the minibody could be preceded by a secretion signal and followed by a membrane anchor, such as the one encoded by the last 37 amino acids of DAF-1 (Rice et al., 1992, Proc. Natl. Acad. Sci. 89:5467-5471). This could be flanked by recombinase sites (e.g., FRT sites) to allow the production of secreted proteins following passage of the library through a recombinase expressing host.

In one embodiment of the present invention, these cassettes would reside at the position normally occupied by the cDNA in the sense-expression vectors described above. In an amber suppressor strain of bacteria and in the presence of helper phage, these vectors would produce a relatively conventional phage display library which could be used exactly as has been previously described for conventional phage display vectors. Recovered phage that display affinity for the selected target would be used to infect bacterial hosts of the appropriate genotype (i.e., expressing the desired recombinases depending upon the cassettes that must be removed for a particular application). For example for an intracellular peptide display, any bacterial host would be appropriate (provided that splice sites are used to remove pelB in the mammalian host). For a secreted display, the minibody vector would be passed through bacterial cells that catalyze the removal of the DAF anchor sequence. Plasmids prepared from these bacterial hosts are used to produce virus for assay of specific phenotypes in mammalian cells.

In some cases, if the target is unknown the phage display step could be skipped and the vectors could be used for intracellular or extracellular random peptide display directly. The advantage of these vectors over conventional approaches is their flexibility. The ability to functionally test the peptide sequence in mammalian cells without additional cloning or sequencing steps makes possible the use of much cruder binding targets (e.g., whole fixed cells) for phage display. This is made possible by the ability to do a rapid functional selection on the enriched pool of bound phages by conversion to retroviruses that can infect mammalian cells.

I) Gene Trapping Vectors

Described herein are forms of the integrating viral vectors, such as replication-deficient retroviral gene, which can be engineered as gene trapping vectors. Such gene trapping vectors contain reporter sequences which, when integrated into an expressed gene, “tag” the expressed gene, allowing for the monitoring of the gene's expression, for example, in response to a stimulus of interest. The gene trapping vectors of the invention can be used, for example, in conjunction with the gene trapping-based methods of the invention for the identification of mammalian genes which are modulated in response to specific stimuli.

The replication-deficient retroviral gene trapping vectors of the invention can comprise: (a) a 5′ LTR; (b) a promoterless 3′ LTR (a SIN LTR); (c) a bacterial Ori; (d) a bacterial selectable marker; (e) a selective nucleic acid recovery element for recovering nucleic acid containing a nucleic acid sequence from a complex mixture of nucleic acid; (f) a polylinker; (g) a mammalian selectable marker; and (h) a gene trapping cassette. In addition, those elements necessary to produce a high titer virus are required. Such elements are well known to those of skill in the art and contain, for example, a packaging signal.

The bacterial Ori, bacterial selectable marker, selective nucleic acid recovery element, polylinker, and mammalian selectable marker are located between the 5′ LTR and the 3′ LTR. The bacterial selectable marker and the bacterial Ori are located in close operative association in order to facilitate nucleic acid recovery, as described below. The gene trapping cassette element is located within the 3′ LTR.

The 5′ LTR, bacterial selectable marker and mammalian selectable marker are as described in Section 5.1, above. The selective nucleic acid recovery element is as the proviral recovery element described above.

The 3′ LTR contains the gene trapping cassette and lacks a functional LTR transcriptional promoter.

The gene trapping cassette can comprise from 5′ to 3′: (a) a nucleic acid sequence encoding at least one stop codon in each reading frame; (b) an internal ribosome entry site; and (c) a reporter sequence. The gene trapping cassette can further comprise, upstream of the stop codon sequences, a transcriptional splice acceptor nucleic acid sequence.

The inclusion of the IRES sequence in the gene trapping vectors of the present invention offers a key improvement over conventional gene trapping vectors. The IRES sequence allows the vector to land anywhere in the mature message to create a bicistronic transcript, this effectively increases the number of integration sites that will report promoters by a factor of at least 10.

J) Retroviral and pEHRE Vector Derivatives

Described herein are derivatives of the retroviral vectors of the invention, including libraries, retroviral particles, integrated proviruses and excised proviruses. Also described herein are derivatives of the pEHRE vectors of the invention, including libraries, cells and animals containing such episomal vectors.

The compositions of the present invention further include libraries comprising a multiplicity of the retroviral and/or pEHRE vectors of the invention, said vectors further containing cDNA or gDNA sequences. A number of libraries may be used in accordance with the present invention, including but not limited to, normalized and non-normalized libraries for sense and antisense expression; libraries selected against specific chromosomes or regions of chromosomes (e.g., as comprised in YACs or BACs), which would be possible by the inclusion of the f1 origin; and libraries derived from any tissue source; and genomic libraries constructed using the BAC/pEHRE vectors of the invention.

The compositions of the present invention still further include retrovirus particles derived from the retroviral vectors of the invention. Such retrovirus particles are produced by the transfection of the retrovirus vectors of the invention into retroviral packaging cell lines, including, but not limited to, the novel retroviral packaging cell lines of the invention.

The compositions of the invention additionally include provirus sequences derived from the retrovirus particles of the invention. The provirus sequences of the invention can be present in an integrated form within the genome of a recipient mammalian cell, or may be present in a free, circularized form.

An integrated provirus is produced upon infection of a mammalian recipient cell by a retrovirus particle of the invention, wherein the infection leads to the production and integration into the mammalian cell genome of the provirus nucleic acid sequence.

The circularized provirus sequences of the invention are generally produced upon excision of the integrated provirus from the recipient cell genome.

The compositions of the present invention still further include cells containing the retroviral or pEHRE vectors of the invention. Such cells include, but are not limited to the packaging cell lines described, below. Additionally, the compositions of the invention include transgenic animals containing the retroviral or pEHRE vectors of the invention, including, preferably, animals containing vectors from which sequences (either sense or antisense) are expressed in one or more cells of the animal.

H) Transcription Profile

The present invention further provides a novel method to study transcription profile of a given biological sample, such as a cell in a specific state. This can be used to compare transcription profiles of different biological samples. For example, the transcription profile of cells from normal and disease tissues, and cells from the same disease tissue before and after certain treatments, etc. According to the present invention, mRNA from the given biological sample can first be isolated and reverse-transcribed into cDNA. Rolling circle amplification can then be employed to amplify the resulting cDNA for use in transcription profile analysis.

The present invention also provides a method to identify modulators of certain signal transduction pathways. According to the present invention, transcription profiles of certain signaling components before and after treatment by certain test compounds can be obtained using methods described above, and modulators of any specific signaling components can be identified by comparing the resulting transcription profiles.

4.4. Packaging Cell Lines

A major prerequisite for the use of retroviruses is to ensure the safety of their use, particularly with regard to the possibility of the spread of wild-type virus in the cell population.

Retroviral packaging functions comprise gag/pol and env packaging functions. gag and pol provide viral structural components and env functions to target virus to its receptor. Env function can comprise an envelope protein from any amphotropic, ecotrophic or xenotropic retrovirus, including but not limited to MuLV (such as, for example, an MuLV 4070A) or MoMuLV. Env can further comprise a coat protein from another virus (e.g., env can comprise a VSV G protein) or it can comprise any molecule that targets a specific cell surface receptor.

The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding one of the subject CCR-proteins, rendering the retrovirus replication defective. The replication defective retrovirus is then packaged into virions which can be used to infect a target cell through the use of a helper virus by standard techniques.

Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm. See for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573). Such prior art systems can be used to package the retroviral-based vectors described above.

However, we have created second-generation retrovirus producer lines for the generation of helper free ecotropic and amphotropic retroviruses. The lines are based on the use of the above-referenced episomal vectors to create a stable, episomal expression system providing the various packaging functions required for packaging replication-deficient retroviral vectors. Salient features of the resulting packaging cell lines are discussed in greater detail below, and include the long term stability of the line, the high titre production of the cell line, and the ability to use cell-lines which are also highly transfectable by such standard techniques as calcium phosphate mediated transfection or lipid-based transfection protocols, e.g., the cells can be highly amenable to transfection with the proviral vectors.

Previously, first-generation producer system were established using 293T cells as a packaging system for helper-free retroviral production. Into 293T cells were placed defective constructs capable of producing gag-pol, and envelope protein for ecotropic and amphotropic viruses. These lines were called BOSC23, and Bing, respectively. See, for example, Pear et al. (1993) PNAS 90:8392. The utility of these lines was that one could produce small amounts of recombinant virus transiently for use in small-scale experimentation. The lines offered advantages over previous stable systems in that virus could be produced in days rather than months. However, two problems are apparent with these and other packaging cell lines in use.

First, these cells are often unstable and need vigilant checking for retroviral production capacity. Second the structure of the vectors used for protein production were not considered fully safe for helper virus production do not possible homologous recombination events between the expression vector of the packaging cell line and retroviral vector

To overcome these obstacle, we have made several improvements. First, we added the facility to monitor gag-pol and/or env production on a cell-by-cell basis by introducing an IRES-marker gene which as part of a polycistronic construct with the gag-pol and/or env coding sequences. Thus, marker gene expression is a direct reflection of expression of the polycistron, and accordingly of the gag-pol and/or env genes. In addition to being a valuable selection tool for early passage of packaging cells, this marker system can also be used to monitor the stability of the producer cell population over time, particularly with respect to it's ability to produce virion proteins. As described below, by proper selection of the marker gene, its expression in the cells can be readily monitored, and utilized to select cells, by flow cytometry.

Second, for the virion protein coding sequence, e.g., both the gag-pol and envelope constructs, non retroviral promoters were used to minimize recombination potential. In preferred embodiments, one could go so far as to even use different promoters for gag-pol and envelope so as to further minimize their inter-recombination potential.

By this technique, several packaging cell lines were created. As described in the appended examples, the envelope coding sequences, Gag-pol and env, were each individually introduced as part of tricistronic messages with a drug selection marker (such as hygromycin) and a FACS tag as the co-selectable markers. The illustrative line LinX, is capable of carrying such episomes for long-term stable production of retrovirus. These lines are readily testable by flow cytometry for stability of envelope expression by way of the FACS tags. Indeed, after more than 60 weeks, the linX line appears more stable than the first-generation line BOSC. Moreover, the subject packaging lines can also be used to transiently produce virus in a few days. Thus, these new lines are fully compatible with transient, episomal stable, and library generation for retroviral gene transfer experiments. Thus, we have provided a means to deliver large libraries of retroviruses into nearly any mammalian cell type, e.g., mouse or human. The viral titre can be to a level, e.g., infectious titers in the range of 10⁵-10⁷/ml or greater, which permits the sampling of complex nucleic acid libraries with enough dynamic range that even relatively rare species in the library have some reasonable chance of being expressed in infected cells.

Thus, one is provided with such viral preparations as purified virus, conditioned media, and/or packaging cell lines producing infectious virus. When working with non-adherent cells, one has the choice of infecting by adding the retroviral supernatant directly to the cells or co-cultivating the non-adherent cells with the retroviral producer cells. The advantage of the latter is that there is ongoing retroviral production; however, this must be weighed against the disadvantage of harvesting producer cells together with the target cells.

Thus, in a preferred embodiment, a retroviral packaging cell line containing a tricistronic expression cassette is used as a founder line for selection of novel efficient, stable retroviral packaging cell lines. The tricistronic message cassette comprises a gene sequence important for efficient packaging of retroviral-derived nucleic acid into functional retroviral particles in operative association with a selectable marker and a quantifiable marker. The gene sequence, the selectable marker and the quantifiable marker are transcribed onto a single message whose expression is controlled by a single set of regulatory sequences. In such an embodiment, the gene sequence important for packaging can represent, for example, a gal/pol or an env gene sequence.

In an alternative embodiment, the retroviral packaging cell line contains a polycistronic expression cassette comprising at least two gene sequences important for efficient packaging of retroviral-derived nucleic acid into functional retroviral particles in operative association with a selectable marker and a quantifiable marker. The gene sequences, the selectable marker and the quantifiable marker are transcribed onto a single message whose expression is controlled by a single set of regulatory sequences. For example, in such an embodiment the gene sequences important for packaging can represent gag/pol and env gene sequences.

The polycistronic, such as, for example, tricistronic, message approach allows for a double selection of desirable packaging cell lines. First, selection for the selectable marker ensures that only those cells expressing the gene sequence important for packaging are selected for. Second, those cells exhibiting the highest level of quantifiable marker (and, therefore, exhibiting the highest level of expression of the gene sequence important for packaging) can be selected.

In a variation of the above embodiment, cell lines containing greater than one polycistronic, e.g., tricistronic, message cassette can be utilized. For example, one message cassette comprising a first gene sequence important for retroviral packaging, a first selectable marker and a first quantifiable marker can be utilized to select for the greatest expression of the first gene sequence, while a second message cassette comprising a second gene important for efficient retroviral packaging, a second selectable marker and a second quantifiable marker can be utilized to select for the greatest expression of the second gene sequence, thereby creating a packaging cell line which is optimized for both the first and the second gene sequences important for packaging.

The quantifiable marker is, for example, any marker gene described above that can be quantified by florescence activated cell sorting (FACS) methods, e.g., a FACS tag. Such a quantifiable marker can include, but is not limited to, any cell surface marker, such as, for example, CD4, CD8 or CD20, in addition to any synthetic or foreign cell surface marker. Further, such a quantifiable marker can include an intracellular fluorescent marker, such as, for example, green fluorescent protein. Additionally, the quantifiable marker can include any other marker whose expression can be measured, such as, for example, a beta galactosidase marker.

The selectable marker chosen can include, for example, any selectable drug marker or the like described above, including, but not limited to hygromycin, blasticidin, neomycin, puromycin, histidinol, zeocin and the like.

High level expression can be achieved by a variety of means well known to those of skill in the art. For example, expression of sequences encoding viral functions can be regulated and driven by regulatory sequences comprising inducible and strong promoters including, but not limited to, CMV promoters.

Alternatively, high copy numbers of polycistronic cassettes can be achieved via a variety of methods. For example, stable genomic insertion of high copy numbers of polycistronic cassettes can be obtained. In one method, extrachromosomal cassette copy number can first be achieved, followed by selection for stable high-copy number insertion. For example, extrachromosomal copy number can be increased via use of SV40 T antigen and SV40 origin of replication in conjunction with standard techniques well known to those of skill in the art.

High stable extrachromosomal cassette copy number can also be achieved. For example, stable extrachromosomal copy number can be increased by making the polycistronic cassettes part of an extrachromosomal replicon derived from, for example, bovine papilloma virus (BPV), human papovavirus (BK) or Epstein Barr virus (EBV) which maintain stable episomal plasmids at high copy numbers (e.g., with respect to BPV, up to 1000 per cell) relative to the 5-10 copies per cell achieved via conventional transfections. In this method the cassettes remain episomal, i.e., there is no selection for integration.

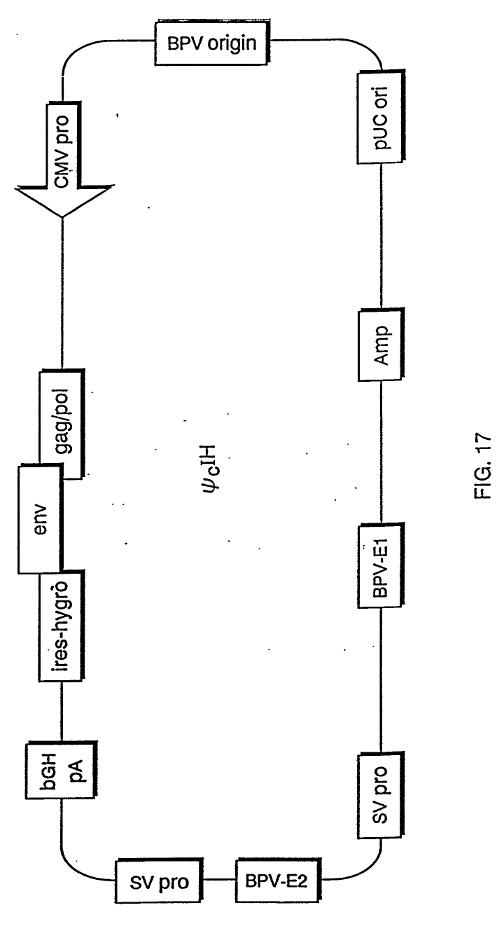

The preferred embodiment for such achieving and utilizing such high level, stable extrachromosomal copy number employs the pEHRE vectors of the invention. FIGS. 17-22 depict pEHRE vectors designed for use in such packaging cell lines. In each of these vectors, the E1 and E2 coding sequences are BPV sequences, and are in operative association with individual SV40 promoters. E1 is transcribed as part of a polycistronic message along with the selectable marker, hygro. In this embodiment, the replication cassette further comprises an SV40 pA site downstream of the IRES-marker. Further, the MO and MME sequences are BPV-derived (in the figure, both of these sequences are illustrated as “BPV origin”).

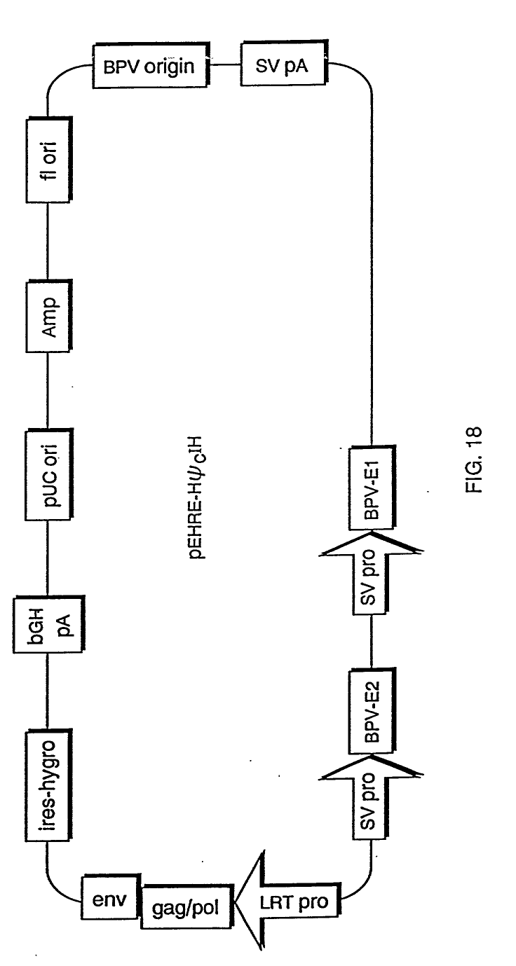

The pEHRE vectors depicted in FIGS. 17 and 18, termed ψ_{c}IH and pEHRE-ψ_{c}IH, respectively, represent two different embodiments of pEHRE vectors whose expression cassette expresses a polycistronic gag/pol env message. The FIG. 17 expression cassette comprises a CMV promoter which is operatively attached to gag/pol, env coding sequences, which are operatively attached to an IRES-hygro construct, which is, in turn, operatively attached to a bGH poly-A site. The FIG. 18 expression cassette is identical to that of FIG. 17, except the promoter utilized is an LTR promoter.

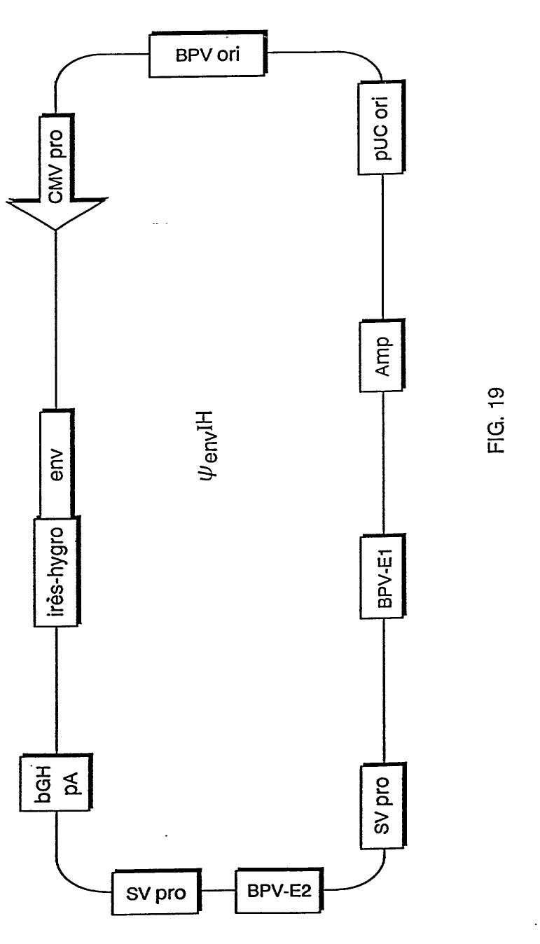

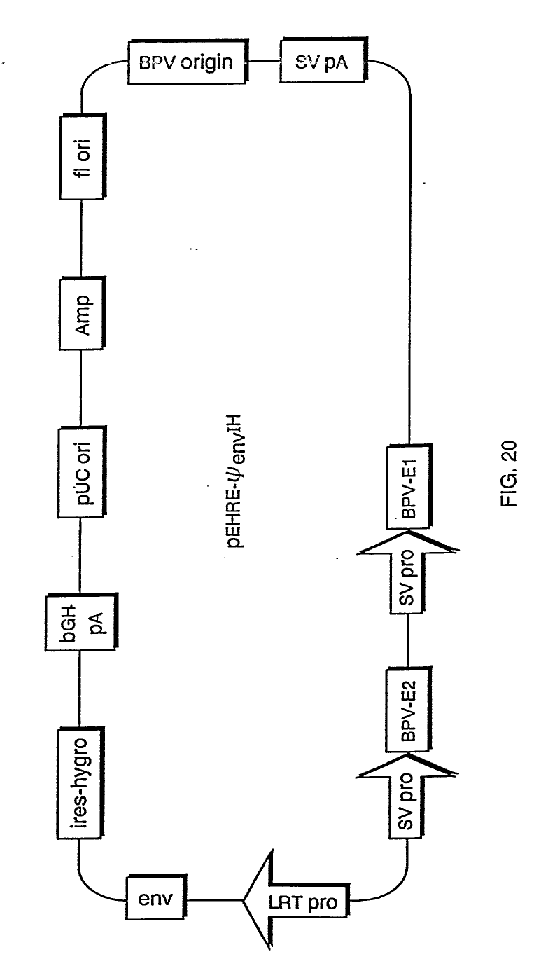

The pEHRE vectors depicted in FIGS. 19 and 20, termed ψₑₙᵥIH and pEHRE-ψₑₙᵥIH respectively, represent two different embodiments of pEHRE vectors whose expression cassette expresses an env message. The FIG. 19 expression cassette comprises a CMV promoter which is operatively attached to an env coding sequence, which is operatively attached to an IRES-hygro construct, which is, in turn, operatively attached to a bGH poly-A site. The FIG. 20 expression cassette is identical to that of FIG. 19, except the promoter utilized is an LTR promoter.

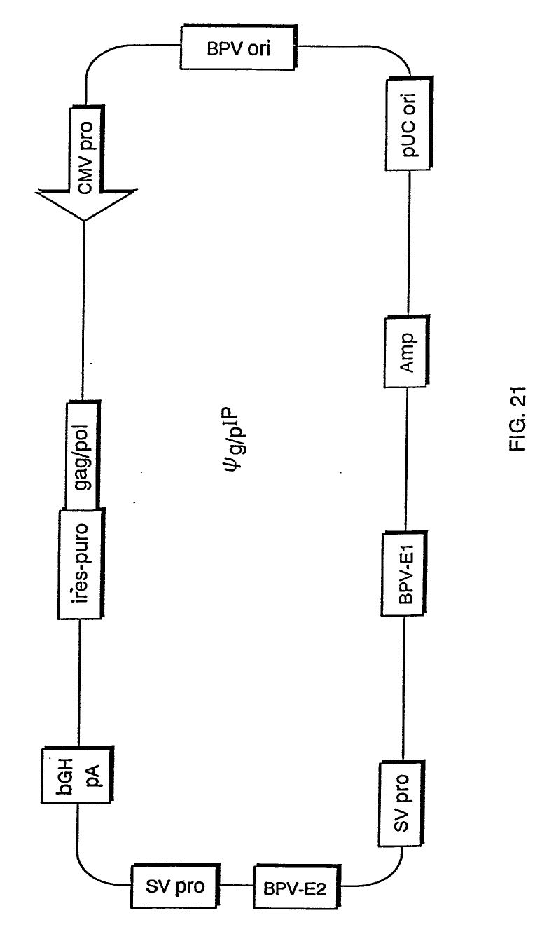

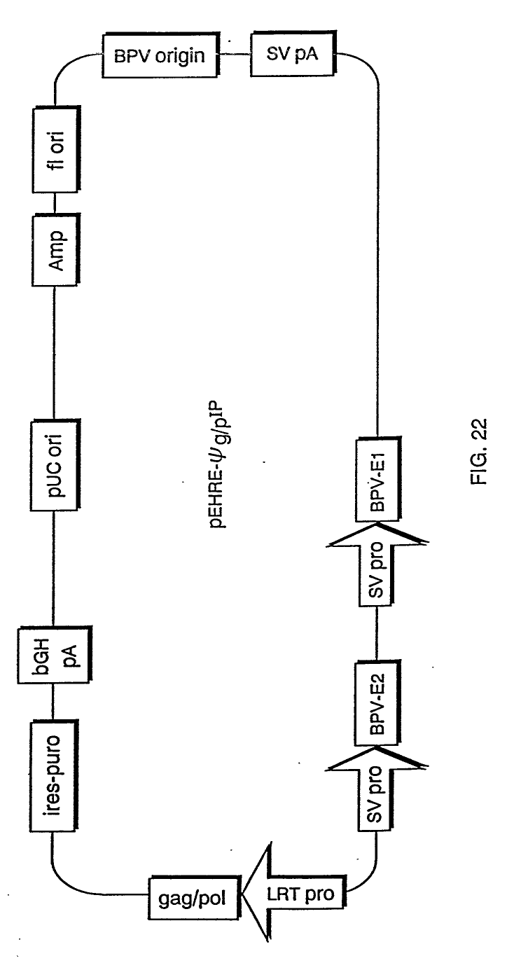

The pEHRE vectors depicted in FIGS. 21 and 22, termed ψ_{g/p}IH and pEHRE-ψ_{g/p}IH, respectively, represent two different embodiments of pEHRE vectors whose expression cassette expresses a polycistronic gag/pol message. The FIG. 21 expression cassette comprises a CMV promoter which is operatively attached to an gag/pol coding sequence, which is operatively attached to an IRES-hygro construct, which is, in turn, operatively attached to a bGH poly-A site. The FIG. 22 expression cassette is identical to that of FIG. 21, except the promoter utilized is an LTR promoter.

Among the cell lines which can be used in connection with pEHRE vectors to produce packaging cell lines are cells that express replication-competent T antigen, such as, for example, COS cells. COS cells express an SV40 T antigen that is capable of promoting replication from the SV40 origin. With respect to packaging cell lines, this can be exploited, first, to allow amplification of replication-deficient retroviral vectors. In this way, expression of retroviral RNA will be increased and higher titers should result, in that it appears that retroviral RNA abundance is the limiting factor for titers in most packaging cell lines. An alternative mechanism for increasing levels introduces a PV, preferably BPV Ori, as described for the pEHRE vectors of the invention, into the retroviral vectors described herein.

The presence of T-antigen can also be utilized to allow amplification of helper functions. This can be accomplished by including an SV40 origin of replication within the pEHRE vectors to achieve higher level expression of helper functions in replication-competent T antigen expressing cells.

Thus, the presence of T-antigen in COS cells can be exploited both to increase the levels of viral genomic RNA and to increase levels of helper functions. In the event that runaway replication of viral genomic RNA is toxic or saturates the packaging system, copy number of the retroviral vectors can be suppressed by the inclusion of BPV sequences just as are copy numbers of the vectors carrying the helper functions.

High cassette copy numbers can also be achieved via gene amplification techniques. Such techniques include, but are not limited to, gene amplification driven by extrachromosomal replicons derived from, for example, BPV, BK, or EBV, as described above. Alternatively, the polycistronic, e.g., tricistronic, message cassettes can further comprise a gene amplification segment including, but not limited to, a DHFR or an ADA segment, which, when coupled with standard amplification techniques well known to those of skill in the art, can successfully amplify message cassette copy number.

The novel retroviral packaging cell lines of the invention can incorporate further modifications which optimize expression from retroviral LTR promoters. In one embodiment, the cell lines exhibit enforced expression of transcription factors that are known to activate retroviral LTR-driven expression in murine T cells. Such transcription factors include, but are not limited to, members of the ets family, cbf (e.g., cbf-a and cbf-b), CTF/NF-1c, glucocorticoid receptor, GRE, NF1, C/EBP, LVa, LVb, and LVc. Retroviral packaging cell lines of this embodiment are designed to more efficiently produce, for example, murine leukemia virus-derived retroviral particles, including but not limited to, Moloney murine leukemia virus (MoMuLV)-derived retroviral particles.

Packaging cell lines with a capacity for increased transcription from the MuMoLv LTR can also be selected in a genetic screen which is executed as described in section 5.7, below. A representative selection scheme begins with a precursor cell line containing a quantifiable marker whose expression is linked to a MoMuLV LTR. Preferably, such an LTR/quantifiable marker construct is excisable. As such, the construct can further comprise an excision element which is equivalent to the proviral excision element described, above.

Precursor cells are infected with a cDNA library derived from murine T-cells. Cells with increased expression, as assayed by the expression of the quantifiable marker, are then identified. Recovery of the library DNA from such cells then identifies gene sequences responsible for such increased expression rates.

The resulting packaging cell lines produced via such a selection scheme exhibit an expression pattern of genes encoding retroviral regulatory factors which closely resembles a murine T-cell pattern of expression for such factors.

Packaging cell lines can be developed which express gag, pol and/or env proteins modified in a manner that promotes an increased viral titer and/or infectivity range. For example, MuLV-based viruses are limited to the infection of proliferating cells. The block to MuLv infection is at the level of entry of the preintegration complex into the nucleus. The complex remains cytoplasmic until dissolution of the nuclear envelope during cell division. Lentiviruses escape this block by incorporating a nuclear targeting signal into the viral capsid. This signal however, must also allow targeting of capsid proteins for assembly at the cytoplasmic face of the cell membrane during viral assembly and budding. This problem is resolved by the fact the nuclear targeting signal of lentiviral capsids is conditional.

In order to overcome the block to MuLv infection of nonproliferating cells, nuclear targeting signals can be incorporated into MuLv virons during assembly in the packaging cell lines of the invention. For example, modified gag proteins can be expressed by the packaging cell lines which can, at low levels, become incorporated into virion capsids during assembly. Nuclear targeting signal sequences are well known to those of skill in the art, and expression of such modified gag proteins can, for example, be via the pEHRE vectors of the invention.

To successfully achieve the goal of creating MuLv virions capable of infecting nonproliferating cells, the gag fusion protein bearing the target signal should be incorporated into the virion capsid as a minority species. Further, the nuclear targeting signal should be a conditional one, such that the fusion is targeted to the nucleus only in infected cells.

In one embodiment of such a modified gag fusion protein, the nuclear targeting signal is one that requires ligand binding for nuclear localization. For example, the glucocorticoid family of receptors have such a ligan-dependent nuclear targeting characteristic.

Alternatively, nuclear targeting of infected cells can be achieved by providing in the infected cell a protein which has affinity for a retroviral capsid (or a tagged retroviral capsid) and also has a nuclear targeting capability, thereby shuttling a virion to the nucleus of infected cells. For example, a single chain antibody can be expressed or introduced which recognizes capsid or capsid tag, wherein the antibody is fused to a nuclear localization signal.

It is also contemplated that similar packaging lines can be derived for adeno-associated viral vectors. For instance, the rep and cap genes are required in trans to provide functions for replication and encapsidation of AAV vectors, and AAV rep and cap coding region can accordingly be provided on the episomal vectors in a manner similar to the retroviral gag-pol and env genes.

4.5. Complementation Screening Methods

Mammalian cell complementation screening methods are described herein. Such methods can include, for example, a method for identification of a nucleic acid sequence whose expression complements a cellular phenotype, comprising: (a) infecting a mammalian cell exhibiting the cellular phenotype with a retrovirus particle derived from a cDNA or gDNA-containing retroviral vector of the invention, or, alternatively, transfecting such a cell with a pEHRE vector of the invention wherein, depending on the vector, upon infection an integrated retroviral provirus is produced or upon transfection an episomal sequence is established, and the cDNA or gDNA sequence is expressed; and (b) analyzing the cell for the phenotype, so that suppression of the phenotype identifies a nucleic acid sequence which complements the cellular phenotype.

The term “suppression”, as used herein, refers to a phenotype which is less pronounced in the presence in the cell expressing the cDNA or gDNA sequence relative to the phenotype exhibited by the cell in the absence of such expression. The suppression may be a quantitative or qualitative one, and will be apparent to those of skill in the art familiar with the specific phenotype of interest.

The present invention also includes methods for the isolation of nucleic acid molecules identified via the complementation screening methods of the invention. Such methods utilize the proviral excision and the proviral recovery elements described above.

In one embodiment of such a method, the proviral excision element comprises a loxP recombination site present in two copies within the integrated provirus, and the proviral recovery element comprises a lacO site, present in the provirus between the two loxP sites. In this embodiment, the loxP sites are cleaved by a Cre recombinase enzyme, yielding an excised provirus which, upon excision, becomes circularized. The excised, circular provirus, which contains the lacO site is recovered from the complex mixture of recipient cell genomic nucleic acid by lac repressor affinity purification. Such an affinity purification is made possible by the fact that the lacO nucleic acid specifically binds to the lac repressor protein.

In an alternative embodiment, the excised provirus is amplified in order to increase its rescue efficiency. For example, the excised provirus can further comprise an SV40 origin of replication such that in vivo amplification of the excised provirus can be accomplished via delivery of large T antigen. The delivery can be made at the time of recombinase administration, for example.

In a preferred embodiment, the excised provirus is amplified by rolling circle amplification (RCA) using an isothermal DNA polymerase, such as the Phi29 DNA polymerase, although other isothermal DNA polymerases can also be used for this purpose.

Alternatively, the whole or partial genomic DNA of isolated positive cells with the desired phenotype can be amplified prior to excision and recovery of the provirus. Such amplification can be done by growing cells, or by PCR amplification of the whole or selected regions (such as the ones encompassing the inserted provirus or other integrated heterologous DNA) of the genomic DNA. In a preferred embodiment, the genomic DNA is first amplified by rolling circle amplification (RCA) using an isothermal DNA polymerase, such as the Phi29 DNA polymerase, although other isothermal DNA polymerases can also be used for this purpose.

In another alternative embodiment, the excised provirus may be recovered by use of a Cre recombinase. For example, the isolated DNA is fragmented to a controlled size. The provirus containing fragments are isolated via LacO/LacI. Following IPTG elution, circularization of the provirus can be accomplished by treatment with purified recombinase.

In a preferred embodiment, the isolated DNA can be amplified before recovery by use of a recombinase such as Cre. The amplification can be either before or after the enrichment/isolation of DNA by proviral recovery element or its equivalents. The amplification is preferably achieved via rolling circle amplification (RCA), although PCR or simply growing cells can also be used.

The coupling of DNA amplification with selective rescue/excision and recovery of heterologous DNA, particularly provirus in phenotypically positive cells, is especially powerful in mammalian cell-based genetic screen. Given the sensitivity of the method, it can be employed to isolated and clone genes from a single positive cell, in a simple and fast reaction under isothermal conditions. This will help to avoid growing back positive cells resulting from the screen—usually a long and tedious process with low efficiency, thus circumventing a traditionally “bottle-neck” step of mammalian cell-based genetic screen.

4.6. Antisense Methods

Antisense genetic suppressor element (GSE)-based methods for the functional inactivation of specific essential or non-essential mammalian genes are described herein. Such methods include methods for the identification and isolation of nucleic acid sequences which inhibit the function of a mammalian gene. The methods include ones which directly assess a gene's function, and, importantly, also include methods which do not rely on direct selection of a gene's function. These latter methods can successfully be utilized to identify sequences which affect gene function even in the absence of knowledge regarding such function, e.g., in instances where the phenotype of a loss-of-function mutation within the gene is unknown.

An inhibition of gene function, as referred to herein, refers to an inhibition of a gene's expression in the presence of a GSE, relative to the gene's expression in the absence of such a GSE. Preferably, the inhibition abolishes the gene's activity, but can be either a qualitative or a quantitative inhibition. While not wishing to be bound by a particular mechanism, it is thought that GSE inhibition occurs via an inhibition of translation of transcript produced by the gene of interest.

The nucleic acid sequences identified via such methods can be utilized to produce a functional knockout of the mammalian gene. A “functional knock-out”, as used herein, refers to a situation in which the GSE acts to inhibit the function of the gene of interest, and can be used to refer to functional knockout cell or transgenic animal.

In one embodiment, a method for identifying a nucleic acid sequence which inhibits the function of a mammalian gene of interest can comprise, for example, (a) infecting a mammalian cell with a retrovirus derived from a GSE-producing retroviral vector containing a nucleic acid sequence from the gene of interest, or, alternatively, transfecting such a cell with a pEHRE-GSE vector of the invention containing a nucleic acid sequence from the gene of interest, wherein the cell expresses a fusion protein comprising an N-terminal portion derived from an amino acid sequence encoded by the gene and a C-terminal portion containing a selectable marker, preferably a quantifiable marker, and wherein an integrated retroviral provirus is produced, or, depending on the vector, an episomal established, that expresses the cDNA or gDNA sequence; (b) selecting for the selectable marker; and (c) assaying for the quantifiable or selectable marker, so that if the selectable marker is inhibited, a nucleic acid sequence which inhibits the function of the mammalian gene is identified.

In one preferred embodiment of this identification method, the fusion protein is encoded by a nucleic acid whose transcription is controlled by an inducible regulatory sequence so that expression of the fusion protein is conditional. In another preferred embodiment of the identification method, the mammalian cell is derived from a first mammalian species and the gene is derived from a second species, a different species as distantly related as is practical.

In a fusion protein-independent embodiment, the nucleic acid encoding the selectable marker can be inserted into the gene of interest at the site of the gene's initiation codon, so that the selectable marker is translated instead of the gene of interest. This embodiment is useful, for example, in instances in which a fusion protein may be deleterious to the cell in which it is to be expressed, or when a fusion protein cannot be made.

The method for identifying a nucleic acid sequence which inhibits the function of a mammalian gene, in this instance, comprises: (a) infecting a mammalian cell expressing a selectable marker in such a fashion with a retrovirus derived from a GSE-producing retroviral vector containing a nucleic acid sequence derived from the gene of interest, or, alternatively, transfecting such a cell with a pEHRE-GSE vector of the invention containing a nucleic acid sequence derived from the gene of interest, wherein, upon infection, an integrated provirus is formed, or, depending on the vector, an episomal sequence is established, and the nucleic acid sequence is expressed; (b) selecting for the selectable marker; and (c) assaying for the selectable marker, so that if the selectable marker is inhibited, a nucleic acid sequence which inhibits the function of the mammalian gene is identified. Selection for the marker should be quantitative, e.g., by FACS.

In an additional embodiment, the gene of interest and the selectable marker can be placed in operative association with each other within a bicistronic message cassette, separated by an internal ribosome entry site, whereby a single transcript is produced encoding, from 5′ to 3′, the gene product of interest and then the selectable marker. Preferably, the sequence within the bicistronic message derived from the gene of interest includes not only coding, but also 5′ and 3′ untranslated sequences.

The method for identifying a nucleic acid sequence which inhibits the function of a mammalian gene, in this instance, comprises: (a) infecting a mammalian cell expressing a selectable marker as part of such a bicistronic message with a retrovirus derived from a GSE-producing retroviral vector containing a nucleic acid sequence derived from the gene of interest, or, alternatively, transfecting such a cell with a pEHRE-GSE vector of the invention containing a nucleic acid sequence derived from the gene of interest, wherein, depending on the vector, upon infection, an integrated provirus is formed, or an episomal sequence is established, and the nucleic acid sequence is expressed; (b) selecting for the selectable marker; and (c) assaying for the selectable marker, so that if the selectable marker is inhibited, a nucleic acid sequence which inhibits the function of the mammalian gene is identified.

In an alternative embodiment, such a method can include a method for identifying a nucleic acid which influences a mammalian cellular function, and can comprise, for example, (a) infecting a cell exhibiting a phenotype dependent upon the function of interest with a retrovirus derived from a GSE-producing retroviral vector containing a test nucleic acid sequence, or, alternatively, transfecting such a cell with a pEHRE-GSE vector of the invention containing a test nucleic acid sequence, wherein, upon infection the an integrated provirus is formed, or, depending on the vector, an episomal sequence is established, and the test nucleic acid is expressed; and (b) assaying the infected cell for the phenotype, so that if the phenotype is suppressed, the test nucleic acid represents a nucleic acid which influences the mammalian cellular function. Such an assay is the same as a sense expression complementation screen except that the phenotype, in this case, is presented only upon loss of function.

The above methods are independent of the function of the gene of interest. The present invention also includes antisense methods for gene cloning which are based on function of the gene to be cloned. Such a method can include a method for identifying new nucleic acid sequences based upon the observation that loss of an unknown gene produces a particular phenotype, and can comprise, for example, (a) infecting a cell with a retrovirus derived from a GSE-producing retroviral vector containing a test nucleic acid sequence, or, alternatively, transfecting such a cell with a pEHRE-GSE vector of the invention containing a test nucleic acid sequence, wherein, upon infection, an integrated provirus is formed, or, depending on the vector, an episomal sequence is established, and the test nucleic acid is expressed; and (b) assaying the infected cell for a change in the phenotype, so that new nucleic acid sequences may be isolated based upon the observation that loss of an unknown gene produces a particular phenotype. Such an assay is the same as a sense expression complementation screen except that the phenotype, in this case, is presented only upon loss of function.

The present invention also includes novel methods for the construction of unidirectional, randomly primed cDNA libraries which can be utilized as part of the function-based methods described above. Such cDNA construction methods can comprise: (a) first strand cDNA synthesis comprising priming the first strand using a nuclease resistant oligonucleotide primer that encodes a restriction site; and (b) second strand cDNA synthesis comprising synthesizing the second strand using an exonuclease deficient polymerase. The nuclease resistant oligonucleotide avoids the removal of a restriction site that marks orientation, thereby allowing for the construction of a unidirectional cDNA random primed cDNA library.

For example, a nuclease resistant chimeric oligonucleotide may be of the general structure: 5′-GCG GCG gga tcc gaa ttc nnn nnn nnn-3′. The modified backbone nucleotides are shown in upper-case, and is generally 4-6 bases, which is followed by one or two restriction sites comprised of normal DNA and nine degenerate nucleotides. A nuclease-deficient polymerase, such as the polymerase from bacteriophage Phi-29, can be used.

The present invention also includes methods for the isolation of nucleic acid molecules identified via the antisense screening methods of the invention. Such methods utilize the proviral excision and the proviral recovery elements, as described above.

In one embodiment of such a method, the proviral excision element comprises a loxP recombination site present in two copies within the integrated provirus, and the proviral recovery element comprises a lacO site, present in the provirus between the two loxP sites. In this embodiment, the loxP sites are cleaved by a Cre recombinase enzyme, yielding an excised provirus which, upon excision, becomes circularized. The excised, circular provirus, which contains the lacO site is recovered from the complex mixture of recipient cell genomic nucleic acid by lac repressor affinity purification. Such an affinity purification is made possible by the fact that the lacO nucleic acid specifically binds to the lac repressor protein.

In an alternative embodiment, the excised provirus is amplified in order to increase its rescue efficiency. For example, the excised provirus can further comprise an SV40 origin of replication such that in vivo amplification of the excised provirus can be accomplished via delivery of large T antigen. The delivery can be made at the time of recombinase administration, for example.

In a preferred embodiment, the excised provirus is amplified by rolling circle amplification (RCA) using an isothermal DNA polymerase, such as the Phi29 DNA polymerase, although other isothermal DNA polymerases can also be used for this purpose.

Alternatively, the whole or partial genomic DNA of isolated positive cells with the desired phenotype can be amplified prior to excision and recovery of the provirus. Such amplification can be done by growing cells, or by PCR amplification of the whole or selected regions (such as the ones encompassing the inserted provirus or other integrated heterologous DNA) of the genomic DNA. In a preferred embodiment, the genomic DNA is first amplified by rolling circle amplification (RCA) using an isothermal DNA polymerase, such as the Phi29 DNA polymerase, although other isothermal DNA polymerases can also be used for this purpose.

4.7. Gene Trapping Methods

The present invention further relates to gene trapping-based methods for the identification and isolation of mammalian genes which are modulated in response to specific stimuli. These methods utilize retroviral particles of the invention to infect cells, which leads to the production of provirus sequences which are randomly integrated within the recipient mammalian cell genome. In instances in which the integration event occurs within a gene, the gene is “tagged” by the provirus reporter sequence, whose expression is controlled by the gene's regulatory sequences. By assaying reporter sequence expression, then, the expression of the gene itself can be monitored.

The gene trapping-based methods of the present invention have several key advantages, including, but not limited to, (1) the presence in the 3′ LTR of a gene trapping cassette that is duplicated upon integration of the provirus into the host genome. This duplication results in the placement of the gene trapping cassette adjacent to genomic DNA such that polymerase entering the virus from an adjacent gene would transcribe the gene trapping cassette before encountering the polyadenylation signal that is present in the LTR. The inclusion of an IRES sequence in the gene trapping cassette allows the fusion between cellular and viral sequence to occur at any point within the mature mRNA, effectively increasing the number of possible integration sites that result in a functionally “tagged” transcript; and (2) the use of a quantifiable selectable marker that can be assessed by live sorting in the FACS, allowing for the isolation of clones that are induced, but also, of clones that tag genes that are repressed.

The term “modulation”, as used herein, refers to an up- or down-regulation of gene expression in response to a specific stimulus in a cell. The modulation can be either a quantitative or a qualitative one.

Gene trapping methods of the invention can include, for example, a method which comprises: (a) infecting a mammalian cell with a retrovirus derived from a gene trapping vector of the invention, wherein, upon infection, an integrated provirus is formed; (b) subjecting the cell to the stimulus of interest; assaying the cell for the expression of the reporter sequence so that if the reporter sequence is expressed, it is integrated within, and thereby identifies, a gene that is expressed in the presence of the stimulus.

In instances wherein the gene is not expressed, or, alternatively, is expressed at a different level, in the absence of the stimulus, such a method identifies a gene which is expressed in response to a specific stimulus.

The present invention also includes methods for the isolation of nucleic acid sequence expressed in the presence of, or in response to, a specific stimulus. Such methods can comprise, for example, digesting the genomic nucleic of a cell which contains a provirus integrated into a gene which is expressed in the presence of, or in response to, the stimulus of interest; and recovering nucleic acid containing a sequence of the gene by utilizing the means for recovering nucleic acid sequences from a complex mixture of nucleic acid.

In one embodiment, the means for recovery is a lacO site, present in the integrated provirus. The digest fragment which contains the lacO site is recovered from the complex mixture of recipient cell genomic nucleic acid by lac repressor affinity purification. Such an affinity purification is made possible by the fact that the lacO nucleic acid specifically binds to the lac repressor protein.

Such methods serve to recover proviral nucleic acid sequence along with flanking genomic sequence (i.e., sequence contained within the gene of interest). The isolated sequence can be circularized, yielding a plasmid capable of replication in bacteria. This is made possible by the presence of a bacterial origin of replication and a bacterial selectable marker within the isolated sequence.

Upon isolation of flanking gene sequence, the sequence can be used in connection with standard cloning techniques to isolate nucleic acid sequences corresponding to the full length gene of interest.

4.8. Embodiments of the Screening Assay

As stated above, the methods of the present invention include methods for the identification and isolation of nucleic acid molecules based upon their ability to complement a mammalian cellular phenotype, antisense-based methods for the identification and isolation of nucleic acid sequences which inhibit the function of a mammalian gene, and gene trapping methods for the identification and isolation of mammalian genes which are modulated in response to specific stimuli.

The compositions of the present invention include replication-deficient retroviral vectors, such as complementation screening retroviral vectors, antisense-genetic suppressor element (GSE) vectors, vectors displaying random peptide sequences, gene trapping vectors, libraries comprising such vectors, retroviral particles produced by such vectors and novel packaging cell lines. The following provides specific embodiments for the utilization of such methods, vectors and compositions for the elucidation of mammalian gene function.

The compositions of the present invention further include pEHRE vectors, such as complementation screening retroviral vectors, antisense-genetic suppressor element (GSE) vectors, vectors displaying random peptide sequences, libraries, cells and animals comprising such vectors, and novel packaging cell lines. The following provides specific embodiments for the utilization of such methods, vectors and compositions for the elucidation of mammalian gene function.

A) Bypass of Conditional Phenotypes

Many phenotypes can be conferred upon mammalian cells in culture by conditional overexpression of known genes (e.g., growth arrest, differentiation). The interference with such phenotypes can be examined by overexpression of sense orientation genes or by functional knock-out (via GSE expression). Examples of this type of screening are given below.

i. Bypass of p53-Mediated Growth Arrest and Apoptosis.

Increases in the level of p53 can cause either growth arrest (generally by cell cycle arrest in G1) or programmed cell death. Cells lines that conditionally overexpressing p53 and contain a p53 functional knock-out will allow for the dissection of both of these processes. In the first case, mouse embryo fibroblasts (MEF) which lack endogenous p53 genes (from p53 knock-out mice) are engineered to conditionally express a fluorescently tagged p53 protein. When activated the fluorescent p53 is localized to the nucleus and enforces cell cycle arrest. Bypass of the arrest can be accomplished by overexpression of sense cDNAs or by expression of GSE fragments. Such a screen might identify components of the p53-degradative pathway, genes that do not affect p53 but allow cell cycle progression even in the presence of p53 and genes that affect p53 localization (p53 is not mutated but is mislocalized in a significant percentage of breast tumors and neuroblastomas). Therefore, use of a fluorescent p53 protein provides information as to the mechanism of bypass.

A very similar cell line can be used to dissect p53-mediated cell death. While p53 alone induces growth arrest in most fibroblasts, combination with certain oncogenes (myc, in particular) causes cell death. MEF cells that conditionally overexpress both myc and p53 are engineered. When activated in combination these genes induce cell death in a substantial fraction of cells. Rescue from this cell death via overexpression of sense oriented cDNAs can be used to identify anti-apoptotic genes (and possible p53-regulators as above). Rescue by GSE expression might identify components of the pathways by which myc and p53 induce cell death (downstream targets) or cellular genes that are required for the apoptotic program.

ii. Bypass of the M1 Component of Cellular Immortalization.

Immortalization of mammalian cells can be divided into two functional steps, M1 and M2. M1 (senescence) can be overcome in fibroblasts by viral oncoproteins that inactivate tumor suppressors, p53 and pRB. SV40 large T antigen is one such protein. Conditionally immortal cells have been derived using temperature sensitive or inducible versions of T-antigen. Upon T inactivation these cells senesce and cease proliferation. The growth of such cells may be rescued by introduction of sense and antisense libraries.

Similar screens can be undertaken with any gene that confers a phenotype upon overexpression. Essentially identical growth-rescue screens can also be undertaken using cytokines that induce growth arrest or apoptosis (e.g., TGF-beta in HMEC or Hep3B cells, respectively).

B) Identification of Cytokines in cis and trans.

Historically, several cytokines have been identified functionally by production in mammalian systems. Specifically, COS cells that express pools of transfected cDNAs have been used to prepare conditioned media that was then tested for the ability to induce growth of factor-sensitive cells. Growth regulatory cytokines may be identified (or survival factors that suppress cell death) by expression of cDNA libraries directly in the target cells. Such an approach has been hampered in the past by the low transfection efficiencies of the target cell types. For example, survival of hematopoietic stem cells is promoted by a variety of known and unknown factors. Therefore, upon infection of such cells with cDNA libraries derived from stromal cells that promote the growth and survival of stem cell populations, selection for surviving infected cells may identify those that carry cDNAs encoding necessary factors. Such factors would be produced in an autocrine mode. While this approach will identify trans-acting factors, cDNA that also act in cis (e.g., by short-circuiting growth-regulatory signal transduction pathways) will also be identified. These can be eliminated by searching for secreted growth regulatory factors using a two-cell system. In this case, one cell type is infected with a library and used as a factory to produce cDNA products, some of which will be secreted proteins. A second cell type that is factor-responsive is then plated over the cDNA expressing cells in a medium (e.g., soft-agar) that restricts diffusion. Responsive cells plated over the producing cells that elaborate the required factor will grow and the appearance of a colony of responsive cells will mark the underlying cells that elaborate the specific factor. The advantage of a two-cell system is more evident in the case where extracellular factors induce growth arrest or terminal differentiation. In such cases, expression in cis would be impractical since selection would be against the population expressing the desired gene. In trans, however, changes in recipient cells can be scored visually and the underlying expressing cells can be rescued for isolation of the desired gene. Similar two cell screens could be developed using the methods of the present invention to screen for factors that promote cell migration or cell-adhesion.

C) Identification of Synthetic Peptides that can Affect Cellular Processes

The present invention provides methods for the identification and isolation of peptides sequences by complementation type screens using vectors capable of displaying random synthetic peptide sequences that interact with a protein of interest in mammalian cells. Conventional screening methods of identifying proteins of interest have been conducted using phage systems and two hybrid screens in yeast. The present invention provides a novel screening method to extend this paradigm to mammalian cells.

i. Intracellular Peptide Display.

As set out above, in another aspect of the present invention the subject vectors can be used for generating peptide display libraries. For embodiments featuring an intracellular peptide library, particular where the peptides are relatively to short, e.g., 5-30 amino acid residues, the peptide can be provided as part of a fusion protein with a conformation-constrained protein (i.e., a protein that decreases the flexibility of the amino and carboxy termini of the protein). In general, conformation-constraining proteins act as scaffolds or platforms, which limit the number of possible three dimensional configurations the peptide or protein of interest is free to adopt. Preferred examples of conformation-constraining proteins are thioredoxin or other thioredoxin-like sequences, but many other proteins are also useful for this purpose. Preferably, conformation-constraining proteins are small in size (generally, less than or equal to 200 amino acids), rigid in structure, of known three dimensional configuration, and are able to accommodate insertions of proteins of interest without undue disruption of their structures. A key feature of such proteins is the availability, on their solvent exposed surfaces, of locations where peptide insertions can be made (e.g., the thioredoxin active-site loop).

As mentioned above, one preferred conformation-constraining protein according to the invention is thioredoxin or other thioredoxin-like proteins. The three dimensional structure of E. coli thioredoxin is known and contains several surface loops, including a distinctive Cys-Cys active-site loop between residues Cys33 and Cys36 which protrudes from the body of the protein. This Cys-Cys active-site loop is an identifiable, accessible surface loop region and is not involved in interactions with the rest of the protein which contribute to overall structural stability. It is therefore a good candidate as a site for prey protein insertions. Both the amino- and carboxyl-termini of E. coli thioredoxin are on the surface of the protein and are also readily accessible for fusion construction.

It may be preferred for a variety of reasons that test peptide be fused within the active-site loop of thioredoxin or thioredoxin-like molecules. The face of thioredoxin surrounding the active-site loop has evolved, in keeping with the protein's major function as a nonspecific protein disulfide oxido-reductase, to be able to interact with a wide variety of protein surfaces. The active-site loop region is found between segments of strong secondary structure and this provides a rigid platform to which one may tether prey proteins. A small heterologous peptide inserted into the active-site loop of a thioredoxin-like protein is present in a region of the protein which is not involved in maintaining tertiary structure. Therefore the structure of such a fusion protein is stable.

Such libraries of random peptide sequences can be expressed from the subject vectors in mammalian cells. Expressed peptides that confer particular phenotypes can be isolated in genetic screens similar to those described above. The cellular targets of these peptides can then be isolated based upon peptide binding in vitro or in vivo.

ii. Extracellular Peptide Display.

It is well established that the interaction between extracellular signaling molecules (e.g., growth factors) and their receptors occurred over large protein surfaces. The present invention provides a novel screen that allows for rapid identification of peptides in mammalian cells by expressing constrained peptides on the surface of receptor-bearing cells and selecting directly for biological function. A synthetic peptide can be displayed in a mammalian system by replacing one flexible loop of a synthetic peptide display vehicle or cassette, the minibody, with a polylinker into which a library of random oligonucleotides encoding random peptides may be inserted. The resulting synthetic chimera can be tethered to the membrane so that it appears on the cell surface by providing a heterologous membrane anchor, such as that derived from the c. elegans decay accelerating factor (DAF). This chimeric protein could then serve as an extracellular peptide display vehicle. Peptide libraries in a retroviral vector could be screened directly for the ability to activate receptors, or screening in vivo could follow a pre-selection of a mini-library by phage display.

To further elaborate, the membrane anchor domain may be any moiety capable of causing attachment to the cell surface. A variety of such moieties are known in the art and include, but are not limited to, transmembrane domains derived from known proteins, a span of hydrophobic amino acid residues sufficient to effect transmembrane spanning, an amino acid sequence that is targeted for post-translational modification by the covalent attachment of lipid molecules and polypeptides having sufficient affinity for a transmembrane protein to effect binding of the molecule to the surface of the cell membrane. Transmembrane domains, both natural and artificial, are known in the art and may be present in multiple copies separated by a sufficient number of amino acid residues to allow multiple membrane spanning by the domains. Typically, a transmembrane domain contains a number of hydrophobic amino acid residues sufficient to span a membrane, and includes at least one and usually several positively charged amino acid residues C-terminal to the hydrophobic amino acids. The positively charged amino acids prevent further transfer of the nascent protein through the membrane. Suitable membrane anchoring domains that function by lipid modification include, but are not limited to, the decay accelerating factor (DAF) which is modified by covalent linkage to glycosyl phosphatidyl inositol (GPI). Such are preferred embodiments and allow for subsequent specific cleavage of the protein from the cell surface.

D) Resistance to Parasite and Viral Infection

Viruses and a number of parasitic organisms require intracellular environments for reproduction. The screens of the present invention may be utilized (e.g., sense overexpression, GSE expression, intracellular peptide display, extracellular peptide display) to identify routes to viral and parasite resistance.

For example, it has recently been demonstrated that resistance to HIV infection can be conferred by expression of a specific mutant gene. The methods of present invention may also be applied to develop a screen for other genes (natural, mutant or synthetic) that confer resistance to HIV infection or that interfere with the viral life cycle.

The methods of the present invention may also be applied to develop a screen for genes that interfere with the viral life cycle of an intracellular parasite, e.g., plasmodium.

E) Identification of Drug-screening Targets for Tumor Cells that Lack Specific Tumor Suppressors

A number of studies have identified two major tumor suppression pathways which are lost in a high percentage of human tumors. The p53 protein is functionally inactivated in approximately 50% of all tumors and the p16/Rb pathway is affected at an even higher frequency. Loss of these pathways for growth control is one of the most obvious distinctions between normal and tumor cells. Many chemotherapeutic drugs act by inducing cell death, and their selectivity is based upon the fact that tumor cells are proliferating while most of the normal cells in the body are quiescent. The methods of the present invention may also be applied to develop screens to identify gene products whose inactivation induces cell death specifically in cells lacking one or both of the two major tumor suppression pathways. This should provide drug screening targets that could lead to compounds that distinguish cells not based upon their proliferation index but based on their genotype.

Identification of such drug screening targets will depend upon that isolation of GSE sequences that can induce apoptosis specifically in the absence of p53 or in the absence of the p16/Rb pathway or both. Cells which conditionally lack either p53, p16/Rb or both can be prepared using conditional viral oncoproteins. For example, p53 can be conditionally inactivated using an inducible E6 protein or using a temperature sensitive T-antigen that has also lost the ability to bind Rb. Conditional loss of p16/Rb can be accomplished using conditionally expressed E7 or again with a ts-T antigen that is mutant for p53 binding. Such cells will be infected with a GSE library and passaged under conditions where p53 or p16/Rb regulation is intact. Those sequences that induce death in normal cells will be naturally counter-selected. The desired tumor suppression pathway will then be specifically inactivated and apoptotic cells will be purified by magnetic separation techniques that rely on the ability of annexing V to bind to the membrane of apoptotic cells. DNA prepared from apoptotic populations will then be used to rescue viral libraries. Several rounds of such screening should enrich for populations of GSE sequences that induce cell death in response to loss of tumor suppressor function.

F. Identification of Genes Involved in Metastasis (In Vivo Selections)

The methods of the present invention may also be applied to develop screens to identify genes involved in metastasis. There are a number of well-characterized systems in which the ability of tumor cells to metastasize can be studied in vivo. The most common is the mouse footpad microinjection assay. Populations of non-metastatic cells can be infected with sense and antisense libraries. These can be injected into the mouse footpad and metastatic cells can be isolated after outgrowth of remote tumors. Rescue of viruses from such cells can be used to identify genes that regulate the ability of tumor cells to metastasize.

Alternatively, in vitro systems can be set up to study the migratory and/or invasive behavior of certain cells, either established mammalian cell lines or primary cells, in tissue culture setting such as trans-well migration assay. Genes affecting migration/invasion can then be isolated from positive cells obtained from these assays. ps 4.9. Target Cell Genome Amplification

In certain embodiments the method of the invention makes use of methods for amplifying the entire target cell genome. Target cell genome amplification can be achieved by PCR-based methods known in the art. For example, PCT WO 00/17390 (the contents of which are herein incorporated by reference) describes methods of whole genome amplification (WGA) which utilize two artificially designed primers adapted to ends of digested genomic DNA to increase the number of copies of genomic segments. Whole genome amplification (WGA) is a method by which more complex amplifications of the DNA from minute samples are generated (Sun, F, et al., 1995, Nucleic Acids Res. 23(15):3034-3040, Barrett, M. T., et al., 1995, Nucleic Acids Res. 23(17):3488-3492.).

PCT WO 00/17390 describes one approach to effecting such a whole genome amplification. Briefly, the method involves: providing a sample of at least one copy of a target cell genome; and digesting the DNA to be amplified with a restriction endonuclease under conditions suitable to obtain DNA fragments of similar length, wherein said restriction endonuclease is capable of providing 5′ overhangs wherein the terminal nucleotide of the overhang is phosphorylated or 3′ overhangs wherein the terminal nucleotide of the overhang is hydroxylated on said DNA fragments; annealing at least one primer to the DNA fragments where simultaneously or subsequently, oligonucleotides representing a first primer are hybridized to the 5′ overhangs on the DNA fragments of the digestion step and where oligonucleotides representing a second primer hybridize to 3′ overhangs generated by the first primer and wherein said first and second primer are of different length; the second primer is ligated to the 5′ overhangs; and said first primer is removed from said DNA fragments; or simultaneously or subsequently, oligonucleotides representing a first primer wherein the nucleotide at the 5′ terminus is phosphorylated are hybridized to the 5′ overhangs on the DNA fragments of the digestion step and wherein oligonucleotides representing a second primer hybridize with the first primer; and the first and second primers are ligated to the DNA fragments; or oligonucleotides representing the primer are hybridized to the 3′ overhangs so that 5′ overhangs are generated; and the primer is ligated to recessed 5′ ends of said DNA fragments; or oligonucleotides representing the primer are ligated to the 5′ overhangs; filling in generated 5′ overhangs; and finally amplifying the DNA fragments with primers which are capable of hybridizing with the complementary strand of said primer(s) of the annealing step.

However, one significant disadvantage of this PCR-based whole genome amplification method is that it is most suitable for amplifying small genomic fragments, such as those ones generated by restriction endonucleases recognizing 4 base-pairs. The average length of these fragments are only between 200-300 bp (4⁴=256), which is less than most full-length genes. Thus, it is not ideally suited for subsequent recovery of heterologous DNA, such as an inserted provirus. In addition, for efficient PCR, a 20-mer is usually needed as a primer, which is ligated to each end of a genomic fragment to be amplified. Thus, more than 10% (40/300) of the amplified product constitute this artificial primer sequence.

Other methods for effecting amplification of a target cell genome are also available for use in the method of the invention. Several documents relating to these other methods are cited throughout the text of this section. Each of the documents cited herein (including any manufacturer's specifications, instructions, etc.) are hereby incorporated by reference; however, there is no admission that any document cited is indeed prior art of the present invention. PCR (polymerase chain reaction) is an extremely powerful in vitro method for the amplification of DNA, which was initially introduced in 1985 (Saiki (1985), Science 230, 1350-1354). By repeated thermal denaturation, primer annealing and polymerase extension, PCR can amplify a single target DNA molecule to easily detectable quantities. Although PCR was initially applied to amplify a single locus in target DNA, it is increasingly being used to amplify multiple loci simultaneously. Frequently used primers for this general amplification of DNA are those based on repetitive sequences within the genome, which allow amplification of segments between suitable positioned repeats.

Interspersed repetitive sequence PCR (IRS PCR) has been used to create human chromosome- and region-specific libraries (Nelson (1989), Proc. Nat. Acad. Sci. USA 86, 6686-6690). In humans, the most abundant family of repeats is the Alu family, estimated to comprise 900,000 elements in the haploid genome, thus giving an average spacing of 3-4 kb (Hwu (1986), Proc. Nat. Acad. Sci. USA 83, 3875-3879). However, a major disadvantage of IRS-PCR is that repetitive sequences like Alu or L1 are not uniformly distributed throughout the genome. Alu elements, for example, are preferentially found in the light bands of human chromosomes. Therefore, such a PCR method results in a bias toward these regions while other regions are less represented and thus not amplified or an amplification can only be obtained below detectable levels. Furthermore, this technique is only applicable to those species where abundant repeat families have been identified, whereas other species such as Drosophila and less well characterized animals and plants cannot be subjected to this method.

A more general amplification than with ISR-PCR can be achieved with “degenerate oligonucleotide-primed PCR” (DOP-PCR), with the additional advantage of species independence (Telenius (1992), Genomics 13, 718-725). DOP-PCR is based on the principle of priming from short sequences specified by the 3′-end of partially degenerate oligonucleotides used, during initial low annealing temperature cycles of the PCR protocol. Since these short sequences occur frequently, amplification of target DNA proceeds at multiple loci simultaneously. DOP-PCR can be applied for generating libraries containing a high level of single copy sequences, provided pure and a substantial amount of DNA of interest can be obtained, e. g. flow-sorted chromosomes, microdissected chromosome bands or isolated yeast artificial chromosomes (YACs). However, DOP-PCR seems to be not capable of providing a sufficient, uniform amplification of the DNA content of a single cell (Kuukasjdrvi (1997), Genes, Chromosomes & Cancer 18, 94-101).

The sensitivity of PCR allows for the analysis of a specific target DNA in a single cell (Li (1988), Nature 335, 414-417). This led to the development of preimplantation genetic disease diagnosis using single cells from early embryos (Handyside (1989), Lancet 1, 347-349) and genetic recombination analysis using a single sperm (Cui (1989), Proc. Nat. Acad. Sci. USA 86, 9389-9393) or oocyte (Cui (1992), Genomics 13, 713-717). However, in all these cases the single cell can be analyzed only once for a given target sequence and independent confirmation of the genotype of any one cell is impossible.

A method called “primer-extension preamplification” (PEP) is directed to circumvent this problem by making multiple copies of the DNA sequences present in a single cell. PEP uses a random mixture of 15-base fully degenerated oligonucleotides as primers, thereby leading to amplification of DNA sequences from randomly distributed sites. It is estimated that about 78% of the genomic sequences in a single human haploid cell can be copied no less than 30 times (Zhang (1992), Proc. Natl. Acad. Sci. USA 89, 5847-5851). However, up to now, a complete and uniform amplification of a whole genome of a single cell has not been documented with methods such as PEP.

A method called representational difference analysis (RDA) is a subtractive DNA hybridization technique that discovers the differences between paired normal and tumor genomes (Lisitsyn (1993), Science 259, 946-951). The minimal amount of DNA needed for RDA shown is 3 ng, corresponding to=1×10³ cells. Generally, 70% of the genomic sequences can be reproducibly amplified by RDA (Lucito (1998), Proc. Natl. Acad. Sci. USA 95, 4487-4492).

RCA is a promising new method for whole genome amplification. According to the instant invention, genomic DNA from a source (e.g., a single cell) can be isolated using any of the well-known methods, and optionally, the genomic DNA can be “cleaned” by protease digestion using a heat-labile protease, for example, Protease K, which can then be inactivated following heat inactivation. Random primers, preferably random hexamers, is then added and rolling circle amplification is initiated at isothermal conditions using a suitable DNA polymerase (e.g., Phi 29 DNA polymerase). This method is advantageous to the traditional PCR-based amplification in that it is carried out under isothermal conditions, thereby obviate the inconvenience and the need for expensive thermal cycling. In addition, due to the high processivity of the enzyme, relatively long genomic templates can be efficiently amplified. This is particularly useful for subsequent cloning of heterologous DNA from the genomic fragments, such as an inserted provirus. Furthermore, no artificial primer DNA exists in the final amplified genomic DNA.

The same method can also be applied to amplification of cDNA, either cDNA library cloned in plasmids, or linear cDNA synthesized from RNA. For linear cDNA amplification, an optional size-fractionation step might be employed to enrich for substantially full-length cDNA or size-select certain sizes of cDNA.

Any DNA polymerase suitable for RCA, such as those disclosed by the instant application, can be used for this purpose. The size of the random primers largely determine the average distance between two annealed primers, and thus the lower end of the smallest amplified genomic fragments. For example, if random hexamers are used, the smallest fragment is on average 4.1 kb (4⁶=4096). While random pentamer gives an average size of 1 kb (4⁵=1024).

4.10. Methods of Excising the Target Nucleic Acid

The invention provides means for the excision of the target nucleic acid sequence, which is preferably a vector sequence such as a retroviral vector sequence, from its integrated state in a target cell genome as well as from tandem repeats of the target nucleic acid sequence that might be produced, for example, by rolling circle DNA polymerase-mediated replication of the target nucleic acid sequence. Excision of the target nucleic acid sequence can be achieved by any of a number of methods known in the art. Particularly preferred methods for excision are by site-specific recombinase-mediated recombination of vector sequence elements as well as standard methods of excision of target nucleic acid sequences such as by restriction with a restriction endonuclease which cuts inside or immediately outside of the target nucleic acid sequence followed by ligation of the resulting free ends to effect circularization of the target nucleic acid sequence. In the case of restriction enzymes, the excised retroviral sequences can remain linear, or can be circularized by religation.

A) Excision by Recombinase

In certain embodiments, the invention provides for the excision of the target nucleic acid sequence by recombinase-mediated recombination. The retroviral vectors' proviral excision element allows for excision of retroviral provirus from the genome of a recipient cell. The element comprises a nucleotide sequence which is specifically recognized by a recombinase enzyme, a restriction enzyme, or other enzyme or agent capable of selectively cleaving genomic DNA in a sequence-dependent manner. The recombinase enzyme cleaves nucleic acid at its site of recognition in such a manner that excision via recombinase action leads to circularization of the excised nucleic acid molecules.

Any site-specific enzyme may be suitable for use in effecting recombinase-mediated excision in the method of the present invention. For example, a type I topoisomerase or a site-specific recombinase such as lambda integrase, FLP recombinase, P I Cre protein, Kw recombinase, and the like (Pan, et al, J Biol. Chem. 268:3683-3689, 1993; Nunes-Duby, et al, EMBO J. 13:4421-443 0, 1994; Hallet and Sherratt, FEMS Microbio. Revs 21:157-178, 1997; Ringrose, et al, Eur J Biochem 248:903 912, 1997).

Enzyme-assisted site-specific integration systems are known in the art and can be applied to the vector system of the invention to excise the viral DNA. Examples of such enzyme-assisted integration systems include the Cre recombinase—lox target system (e.g., as described in Baubonis, W. and Sauer, B. (1993) Nucl. Acids Res. 21:2025-2029; and Fukushige, S. and Sauer, B. (1992) Proc. Natl. Acad. Sci. U.S.A. 89:7905-7909) and the FLP recombinase—FRT target system (e.g., as described in Dang, D. T. and Perrimon, N. (1992) Dev. Genet. 13:367-375; and Fiering, S. et al. (1993) Proc. Natl. Acad. Sci. U.S.A. 90:8469-8473); the Piv site-specific DNA recombinase from Moraxella lacunata (e.g., described by Lenich et al. (1994) J Bacteriol 176) 4160); Lambda integrase (e.g, Kwon et al. (1997) Science 276:126). It is important to note that the preferred site-specific recombinases for the purposes of excision and excision/amplification of the target nucleic acid sequence are dictated in large part by the identity of the site-specific integration system utilized in the particular vector.

A particularly preferred enzyme-assisted site-specific recombinase for use in the invention is the Kw recombinase, as integrase from Kluyveromyces waltii (see Rignrose et al. (1997) Eur J Biochem 248: 903-12, the contents of which are hereby incorporated by reference). Site-specific recombinases of the integrase family share limited amino acid sequence similarity, but use a common reaction mechanism to recombine distinct DNA target sites. The Kw site-specific recombinase, encoded on the 2 μ-like plasmid pKWS1 from the yeast Kluyveromyces waltii, is able to bind and to recombine its putative DNA target site. Recombination is conservative and the Kw target site has a spacer of seven base pairs. The Kw recombinase is able to mediate recombination in a mammalian cell line.

By “recombinase target site” (RTS) herein is meant a nucleic acid sequence which is recognized by a recombinase for the excision of the intervening sequence. It is to be understood that two RTSs are required for excision. Thus, when the Cre recombinase is used, each RTS comprises a loxP site; when loxP sites are used, the corresponding recombinase is the Cre recombinase. That is, the recombinase must correspond to or recognize the RTSs. When the FLP recombinase is used, each RTS comprises a FLP recombination target site (FRT); when FRT sites are used, the corresponding recombinase is the FLP recombinase.

A number of different site specific recombinase systems can be used, including but not limited to the Cre/lox system of bacteriophage P1, the FLP/FRT system of yeast, the Gin recombinase of phage Mu, the Pin recombinase of E. coli, and the R/RS system of the pSR1 plasmid. The two preferred site specific recombinase systems are the bacteriophage P1 Cre/lox and the yeast FLP/FRT systems. In these systems a recombinase (Cre or FLP) will interact specifically with its respective site-specific recombination sequence (lox or FRT respectively) to invert or excise the intervening sequences. The sequence for each of these two systems is relatively short (34 bp for lox and 47 bp for FRT). Currently the FLP/FRT system of yeast is the preferred site specific recombinase system since it normally functions in a eukaryotic organism (yeast), and is well characterized.

In a preferred embodiment, the recombinase recognition site is located within the 3′ LTR at a position which is duplicated upon integration of the provirus. This results in a provirus that is flanked by recombinase sites.

In an exemplary embodiment, the proviral excision element comprises a loxP recombination site located in the LTR. Contacting Cre recombinase to an integrated provirus derived from the retroviral vector results in excision of the provirus nucleic acid. In the alternative, a mutant lox P recombination site any be used (e.g., lox P511 (Hoess et al., 1986, Nucleic Acids Research 14:2287-2300)) that can only recombine with an identical mutant site.

In yet another preferred embodiment, an FRT recombination site, which is cleavable by a FLP recombinase enzyme, is utilized in conjunction with FLP recombinase enzyme, as described above for the loxP/Cre embodiment. A “Flip Recombination Target site” (FRT) refers to a nucleotide sequence that serves as a substrate in the site-specific yeast flip recombinase system. The FRT recombination region has been mapped to an approximately 65-base pair (bp) segment within the 599-bp long inverted repeats of the 2μ-m circle (a commonly occurring plasmid in Saccharomyces cerevisiae). The enzyme responsible for recombination (FLP) is encoded by the 2-μm circle, and has been expressed at high levels in human cells. FLP catalyzes recombination within the inverted repeats of the molecule to cause intramolecular inversion. FLP can also promote efficient recombination between plasmids containing the 2-μm circle repeat with very high efficiency and specificity. See, e.g., Jayaram (1985) Proc. Natl. Acad. Sci. USA 82:5875-5879; and O'Gorman (1991) Science 251:1351-1355. A “minimum FRT site” (e.g., a minimal FLP substrate) has been described in the art and is defined herein as a 13-bp dyad symmetry plus an 8-bp core located within the 65-bp FRT region. Jayaram et al., supra. Both FRT sites and FLP expression plasmids are commercially available from Stratagene (San, Diego, Calif.).

In still another preferred embodiment, an R recombinase site and R recombinase from Zygosaccharomyces rouxii can be utilized, as described above, in place of the loxP/Cre embodiment. EC 2.7.7.-(R′ recombinase). See also Chen et al. (1991) PNAS 88: 5944.

Another suitable enzyme for use in the invention method is a type II or a type I topoisomerase. For example, vaccinia DNA topoisomerase I binds to duplex DNA and cleaves the phosphodiester backbone of one strand. The enzyme exhibits a high level of sequence specificity, akin to that of a restriction endonuclease. Cleavage occurs at a consensus pentapyrimidine element ′-(C/T)CCTT in the scissile strand. In the cleavage reaction, bond energy is conserved via the formation of a covalent adduct between the 3′ phosphate of the incised strand and a tyrosyl residue of the protein. Vaccinia topoisomerase can religate the covalently held strand across the same bond originally cleaved (as occurs during DNA relaxation) or it can religate to a heterologous acceptor DNA and thereby create a recombinant molecule.

When the substrate is configured such that the scissile bond is situated near (within 10 basepairs of) the 3′ end of a DNA duplex, cleavage is accompanied by the spontaneous dissociation of the downstream portion of the cleaved strand. The resulting topoisomerase-DNA complex, containing a 5′ single-stranded tail, can religate to an acceptor DNA if the acceptor molecule has a 5′ OH tail complementary to that of the activated donor complex. The use of vaccinia topoisomerase type I for cloning is described in detail in copending U.S. patent application Ser. No. 08/358,344, filed Dec. 19, 1994, incorporated by reference herein in its entirety.

B) Excision by Restriction and Religation

In yet another alternative embodiment, a rare-cutting restriction enzyme (e.g., Not I) may be used in place of the recombinase site. The recovered DNA would be digested with Not I and then recircularized with ligase. In this embodiment, the Not I site is included in the vector next to loxP. In other embodiments, the restriction enzyme can be 8 or higher base cutter, e.g., requires at least 8 basepairs for specificity.

Restriction followed by recircularization to effect excision of a target nucleic acid sequence is a method well known in the art and commonly employed to effect plasmid rescue. For example, plasmid rescue experiments typically consist of digesting integrated transgenes outside of the required genetic elements with restriction enzymes, circularizing the restriction fragments, electroporating bacteria, and then selecting (e.g. for antibiotic resistance encoded by the target nucleic acid sequence such as ampicillin resistance, kanamycin resistance (neo rescue), etc.). In the case of neo rescue experiments, one can also grow the colonies on Xp gal plates and measure the percentage of recovered plasmids with corrected transgenes by blue/white colony screening. These in vitro experiments demonstrate that all the constructs required for gene correction are working properly.

In certain preferred embodiments of the invention, yeast homothallic switching endonuclease (HO endo), a sequence-specific double-strand nuclease involved in mating-type switching, is employed for DNA cleavage of the target nucleic acid sequence. HO endo contains discrete functional domains: a N-terminal nuclease and a C-terminal DNA-binding domain, thereby allowing construction of a chimeric nuclease with the cutting site distinct from the original HO recognition sequence. The expression of the nuclease can be engineered to be controlled by a tightly regulated, inducible promoter (see Liang and Girard (1999) Methods 17: 95-103). Methods of using the HO endo (also known as SceI) restriction endonuclease from Saccharomyces cerevisiae for cloning manipulations, such as in plasmid rescue/restriction religation methods, are well known in the art (see e.g. Viret (1993) Biotechniques 14: 325, incorporated herein by reference).

4.11. Amplification and Cloning of the Target Nucleic Acid

Methods for polymerase-mediated amplifications are well known in the art. Particularly preferred methods for amplification employ Phi 29 DNA polymerase, although polymerases, for example DNA polymerases of dsDNA viruses, are understood to be included within the scope of the invention.

Rolling-circle amplification (RCA) driven by DNA polymerase can replicate circularized oligonucleotide probes with either linear or geometric kinetics under isothermal conditions. In the presence of two primers, one hybridizing to the + strand, and the other, to the − strand of DNA, a complex pattern of DNA strand displacement ensues that generates 10⁹ or more copies of each circle in 90 minutes, enabling detection of point mutations in human genomic DNA. Using a single primer, RCA generates hundreds of tandemly linked copies of a covalently closed circle in a few minutes. If matrix-associated, the DNA product remains bound at the site of synthesis, where it may be tagged, condensed and imaged as a point light source. Linear oligonucleotide probes bound covalently on a glass surface can generate RCA signals, the color of which indicates the allele status of the target, depending on the outcome of specific, target-directed ligation events. As RCA permits millions of individual probe molecules to be counted and sorted using color codes, it is particularly amenable for the analysis of rare somatic mutations. RCA also shows promise for the detection of padlock probes bound to single-copy genes in cytological preparations (see Lizardi et al. (1998) Nat Gen 19: 225, the contents of which are incorporated herein by reference).

RCA has been exploited in a number of biotechnology applications. For example, circularizing oligonucleotide probes (e.g. padlock probes) have the potential to detect sets of gene sequences with high specificity and excellent selectivity for sequence variants, but sensitivity of detection has been limiting. By using a rolling circle amplification (RCA) mechanism, circularized but not unreacted probes can yield a powerful signal amplification. In order for the reaction to proceed efficiently the probes must be released from the topological link that forms with target molecules upon hybridization and ligation. If the target strand has a nearby free 3′ end, then the probe-target hybrids can be displaced by the polymerase used for replication. The displaced probe can then slip off the target strand and a rolling circle amplification is initiated. Alternatively, the target sequence itself can prime an RCA after its non-based-paired 3′ end has been removed by exonucleolytic activity. The Φ29 DNA polymerase is superior to the Klenow fragment in displacing the target DNA strand, and it maintained the polymerization reaction for at least 12 h, yielding an extension product that represents several thousand-fold the length of the padlock probe (see Baner et al. (1998) NAR 26: 5073, the contents of which are incorporated herein by reference).

Phi 29-mediated amplification has been described—see, for example, WO 99/49079 hereby incorporated by reference, which describes Phi 29-mediated amplification of certain padlock probes). Amplification of a circular nucleic acid molecule free in solution by rolling circle amplification reaction may be achieved simply by hybridizing a primer to the circular nucleic acid and providing a supply of nucleotides and a polymerase enzyme. However, certain problems may arise when the circular nucleic acid is not free in solution. DNA polymerases useful in the rolling circle amplification step of RCA must perform rolling circle amplification of primed single-stranded circles. Such polymerases are referred to herein as rolling circle DNA polymerases. For rolling circle amplification, it is preferred that a DNA polymerase be capable of displacing the strand complementary to the template strand, termed strand displacement, and lack a 5′ to 3′ exonuclease activity. Strand displacement is necessary to result in synthesis of multiple tandem copies of the ligated OCP (Open Circle Probe). A 5′ to 3′ exonuclease activity, if present, might result in the destruction of the synthesized strand. It is also preferred that DNA polymerases for use in the disclosed method are highly processive. The suitability of a DNA polymerase for use in the disclosed method can be readily determined by assessing its ability to carry out rolling circle amplification. Preferred rolling circle DNA polymerases are bacteriophage [Phi] 29 DNA polymerase (U.S. Pat. Nos. 5,198,543 and 5,001,050 to Blanco et al.), phage M2 DNA polymerase (Matsumoto et al., Gene 84:247 (1989)), phage [Phi] PRD1 DNA polymerase (Jung et al., Proc. Natl. Acad. Sci. USA 84:8287 (1987)), VENT [r] DNA polymerase (Kong et al., J. Biol. Chem. 268:1965-1975 (1993)), Klenow fragment of DNA polymerase I (Jacobsen et al., Eur. J. Biochem. 45:623-627 (1974)), T5 DNA polymerase (Chatterjee et al., Gene 97:13-19 (1991)), PRD1 DNA polymerase (Zhu and Ito, Biochim. Biophys. Acta. 1219:267-276 (1994)), and T4 DNA polymerase holoenzyme (Kaboord and Benkovic, Curr. Biol. 5:149-157 (1995)). [Phi] 29 DNA polymerase is most preferred.

Strand displacement can be facilitated through the use of a strand displacement factor, such as helicase. It is considered that any DNA polymerase that can perform rolling circle amplification in the presence of a strand displacement factor is suitable for use in the disclosed method, even if the DNA polymerase does not perform rolling circle amplification in the absence of such a factor. Strand displacement factors useful in RCA include BMRF1 polymerase accessory subunit (Tsurumi et al., J. Virology 67(12):7648-7653 (1993)), adenovirus DNA-binding protein (Zijderveld and van der Vliet, J. Virology 68(2):1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

The ability of a polymerase to carry out rolling circle amplification can be determined by using the polymerase in a rolling circle amplification assay such as those described in Fire and Xu, Proc. Natl. Acad. Sci. USA 92:4641-4645 (1995).

Another type of DNA polymerase can be used if a gap-filling synthesis step is used, such as in gap-filling LM-RCA. When using a DNA polymerase to fill gaps, strand displacement by the DNA polymerase is undesirable. Such DNA polymerases are referred to herein as gap-filling DNA polymerases. Unless otherwise indicated, a DNA polymerase referred to herein without specifying it as a rolling circle DNA polymerase or a gap-filling DNA polymerase, is understood to be a rolling circle DNA polymerase and not a gap-filling DNA polymerase. Preferred gap-filling DNA polymerases are T7 DNA polymerase (Studier et al., Methods Enzmol. 185:60-89 (1990)), DEEP VENTS [r] DNA polymerase (New England Biolabs, Beverly, Mass.), and T4 DNA polymerase (Kunkel et al., Methods Enzmol. 154:367-382 (1987)). An especially preferred type of gap-filling DNA polymerase is the Thermus flavus DNA polymerase (MBR, Milwaukee, Wis.). The most preferred gap-filling DNA polymerase is the Stoffel fragment of Taq DNA polymerase (Lawyer et al., PCR Methods Appl. 2(4):275-287 (1993), King et al., J. Biol. Chem. 269(18):13061-13064 (1994)).

A variation of the RCA is call “hyper-branched rolling circle amplification (HRCA)”. Lizardi et al. (Nature Genetics, 19: 225-232, 1998) described in detail of the HRCA process, the entire contents of which are herein incorporated by reference. In brief, two primers are used in HRCA instead of just one that hybridize to the closed padlock probe or circular template. The second primer will bind to each complementary sequence in the tandem single-stranded DNA, initiating sequential primer extension reactions. As each extending primer runs into the product of a downstream primer, strand displacement will ensue, generating single-stranded tandem repeats of the sequence of the original circularized probe. This displaced strand will in turn contain multiple binding sites for the first RCA primer. Thus, alternate-strand copying and strand displacement processes generate a continuously expanding pattern of DNA branches connected to the original circle. Strand displacement also generates a discrete set of free DNA fragments comprising double-stranded pieces of the unit length of a circle, and multiples thereof. If random primers are used for amplification of linear DNA, due to the nature of the reaction, both RCA and HRCA will function to give rise to the final amplification product.

Any RCA enzymes in theory can be used for HRCA. Preferred HRCA DNA polymerase is exo(−) Vent DNA polymerase, the large fragment of Bst DNA polymerase (New England Biolabs, Beverly, Mass.), the Sequenase 2.0 variant of T7 DNA polymerase, and Phi 29 DNA polymerase. The efficiency of RCA or HRCA may be increased at the presence of proteins that bind ssDNA (single strand DNA), such as the E. coli single-strand binding protein (SSB), phage T4 gene-32 protein. The yield of HRCA reaction can be potentially increased by using polymerase with relatively slow polymerization rates (for example, about 16.5 nt per second, as reported for Vent DNA polymerase) which permits a more efficient use of potential priming sites.

The amplified DNA can be resolved using any of the methods as described in section 4.10 above. The resulting DNA fragments can be cloned using standard molecular biology protocols, such as those described in Current Protocols in Molecular Biology, Ausubel, F. M. et al. (eds.) Greene Publishing Associates, (1989).

Since RCA is a powerful, fast and efficient isothermal DNA amplification method that can achieve 10⁹ fold amplification of the input DNA, it is particularly useful for cloning genes or heterologous DNA fragments directly from a single cell. To illustrate, small quantities of cells obtained from mammalian cell-based genetic screen usually need to be grown back to sufficient quantity for isolation of the genomic DNA and recovery of the heterologous genetic elements. This is a time-consuming process that frequently results in cell loss since many mammalian cells fail to grow at low density. RCA/HRCA make it possible to quickly obtain the genomic DNA from positive cells, thus facilitating efficient cloning of the gene responsible for the phenotypic change. Coupled with excisable provirus, additional rounds of rescreen is possible so that the overall reliability of the system is improved, enabling screening under relatively high experimental backgrounds.

In a preferred embodiment, the excised DNA may be enriched before cloning. The enrichment step is effected by using a DNA recovery element (such as a proviral recovery element) in the target heterologous DNA as described above.

The ability of a polymerase to fill gaps can be determined by performing gap-filling LM-RCA. Gap-filling LM-RCA is performed with an open circle probe that forms a gap space when hybridized to the target sequence. Ligation can only occur when the gap space is filled by the DNA polymerase. If gap-filling occurs, TS-DNA can be detected, otherwise it can be concluded that the DNA polymerase, or the reaction conditions, is not useful for gap-filling using DNA polymerase.

Any RNA polymerase which can carry out transcription in vitro and for which promoter sequences have been identified can be used in the disclosed rolling circle transcription method. Stable RNA polymerases without complex requirements are preferred. Most preferred are T7 RNA polymerase (Davanloo et al., Proc. Natl. Acad. Sci. USA 81:2035-2039 (1984)) and SP6 RNA polymerase (Butler and Chamberlin, J. Biol. Chem. 257:5772-5778 (1982)) which are highly specific for particular promoter sequences (Schenborn and Meirendorf, Nucleic Acids Research 13:6223-6236 (1985)). Other RNA polymerases with this characteristic are also preferred. Because promoter sequences are generally recognized by specific RNA polymerases, the OCP or ATC should contain a promoter sequence recognized by the RNA polymerase that is used. Numerous promoter sequences are known and any suitable RNA polymerase having an identified promoter sequence can be used. Promoter sequences for RNA polymerases can be identified using established techniques.

In the particular case of a padlock primer which is catenated to its target, it might reasonably be expected that the target would inhibit a rolling circle amplification reaction. If the target is circular, then rolling circle amplification of a padlock primer catenated to the target is effectively prevented. Where a target is linear, it is possible in principle for a padlock formed thereon to slide along the target molecule and off the end, thereby becoming free in solution and available for amplification by a rolling circle amplification reaction. The target sequence, the site of binding by the padlock probe, may be digested by exonucleolysis, allowing the remaining target strand to prime rolling circle amplification. The sliding and uncoupling effect is possible to a limited extent even with long linear targets. Thus, converting the circular 7 kb M13 genome by restriction at a single site into a linear 7 kb nucleic acid molecule WO 99/49079 with 3.5 kb upstream and 3.5 kb downstream of the hybridization site of the padlock primer, limited amplification of the padlock primer was possible by rolling circle amplification. Where the target nucleic acid is substantially shorter, e.g. a few tens or hundreds of bases, rolling circle amplification of a padlock primer formed thereon is much more rapid and efficient.

Amplification techniques can be grouped into those requiring temperature cycling (PCR, (Saiki, R. K. et al. (1985), Science 230:1350-54 (incorporated herein by reference)) , ligase chain reaction, (Wu, D. Y. et al.(1989), Genomics 4:560-69; Barringer, K. et al. (1990), Gene 89:117-22; Barany, F. (1991), Proc. Natl. Acad. Sci. USA 88:189-93 (all incorporated herein by reference)), and transcription-based amplification (Kwoh, D. Y. et al. (1989), Proc. Natl. Acad. Sci. USA 86:1173-77 (incorporated herein by reference))] and isothermal systems [self-sustained sequence replication (Guatelli, J. C. et al. (1990), Proc. Natl. Acad. Sci. USA 87:1874-78 incorporated herein by reference)) and a Qβ replicase system (Lizardi, P. M. et al. (1988), Biotechnology 6:1197-1202 (incorporated herein by reference))] [for a comparative review, see Kwoh and Kwoh (Kwoh, D. Y. et al. (1990), Am. Biotechnol. Lab. 8:14-25 (incorporated herein by reference))].

In preferred embodiments, the invention makes use of strand displacement amplification methodologies for amplification of the target nucleic acid sequence. Strand displacement amplification (SDA) is a DNA amplification technique that uses readily available enzymes and does not require temperature cycling. The technique is based upon the ability of a restriction enzyme to nick the unmodified strand of a hemimodified DNA recognition site, and the ability of a 5′-3′ exonuclease-deficient DNA polymerase to extend the 3′ end at the nick and displace the downstream strand. Exponential target DNA amplification is achieved by coupling sense and antisense reactions in which strands displaced from a sense reaction serve as a target for an antisense reaction and vice versa. SDA has been applied to genomic DNA samples from M. tuberculosis and M. bovis using a portion of the IS6110 element as target sequence where amplifications of 10⁶-fold have been achieved (see e.g. Walker et al. (1992) PNAS, USA 89: 392, herein incorporated by reference). Therefore SDA is a valid isothermal alternative for amplifying specific DNA sequences.

In preferred embodiments of the invention, amplification of the target nucleic acid and/or the target genomic DNA is effected by Phi 29 DNA polymerase. Phi 29 DNA replication starts at both DNA ends by a protein priming mechanism. The formation of the terminal protein-dAMP initiation complex is directed by the second nucleotide from the 3′ end of the template. The transition from protein-primed initiation to normal DNA elongation has been proposed to occur by a sliding-back mechanism that is necessary for maintaining the sequences at the Phi 29 DNA ends. Structure-function studies have been carried out in the Phi 29 DNA polymerase. By site-directed mutagenesis of amino acids conserved among distantly related DNA polymerases we have shown that the N-terminal domain of Phi 29 DNA polymerase contains the 3′-5′ exonuclease activity and the strand-displacement capacity, whereas the C-terminal domain contains the synthetic activities (protein-primed initiation and DNA polymerization). Viral protein p6 stimulates the initiation of Phi 29 DNA replication. The structure of the protein p6-DNA complex has been determined, as well as the main signals at the Phi 29 DNA ends recognized by protein p6. The DNA binding domain of protein p6 has been studied. The results indicate that an alpha-helical structure located in the N-terminal region of protein p6 is involved in DNA binding through the minor groove. The Phi 29 protein p5 is the single-stranded DNA binding (SSB) protein involved in Phi 29 DNA replication, by binding to the displaced single-stranded DNA (ssDNA) in the replication intermediates. In addition, protein p5 is able to unwind duplex DNA. The properties of the Phi 29 SSB-ssDNA complex are described. Using the four viral proteins, terminal protein, DNA polymerase, protein p6 and the SSB protein, it was possible to amplify the 19,285-bp Phi 29 DNA molecule by a factor of 4000 after 1 h of incubation at 30 degrees C. The infectivity of the in vitro amplified DNA was identical to that of Phi 29 DNA obtained from virions (see Salas et al. (1995) FEMS Microbiol Rev 17: 73-82).

An amplification target circle, when replicated, gives rise to a long DNA molecule containing multiple repeats of sequences complementary to the amplification target circle. This long DNA molecule is referred to herein as tandem sequences DNA (TS-DNA). TS-DNA contains sequences complementary to the primer complement portion and, if present on the amplification target circle, the detection tag portions, the secondary target sequence portions, the address tag portions, and the promoter portion. These sequences in the TS-DNA are referred to as primer sequences (which match the sequence of the rolling circle amplification primer), spacer sequences (complementary to the spacer region), detection tags, secondary target sequences, address tags, and promoter sequences. Amplification target circles are useful as tags for specific binding molecules.

A rolling circle replication primer (RCRP) is an oligonucleotide having sequence complementary to the primer complement portion of an OCP or ATC. This sequence is referred to as the complementary portion of the RCRP. The complementary portion of a RCRP and the cognate primer complement portion can have any desired sequence so long as they are complementary to each other. In general, the sequence of the RCRP can be chosen such that it is not significantly complementary to any other portion of the OCP or ATC. The complementary portion of a rolling circle amplification primer can be any length that supports specific and stable hybridization between the primer and the primer complement portion. Generally this is 10 to 35 nucleotides long, but is preferably 16 to 20 nucleotides long.

It is preferred that rolling circle amplification primers also contain additional sequence at the 5′ end of the RCRP that is not complementary to any part of the OCP or ATC. This sequence is referred to as the non-complementary portion of the RCRP. The non-complementary portion of the RCRP, if present, serves to facilitate strand displacement during DNA replication. The non-complementary portion of a RCRP may be any length, but is generally 1 to 100 nucleotides long, and preferably 4 to 8 nucleotides long. The rolling circle amplification primer may also include modified nucleotides to make it resistant to exonuclease digestion. For example, the primer can have three or four phosphorothioate linkages between nucleotides at the 5′ end of the primer. Such nuclease resistant primers allow selective degradation of excess unligated OCP and gap oligonucleotides that might otherwise interfere with hybridization of detection probes, address probes, and secondary OCPs to the amplified nucleic acid. A rolling circle amplification primer can be used as the tertiary DNA strand displacement primer in strand displacement cascade amplification.

Primers used for secondary DNA strand displacement are referred to herein as DNA strand displacement primers. One form of DNA strand displacement primer, referred to herein as a secondary DNA strand displacement primer, is an oligonucleotide having sequence matching part of the sequence of an OCP or ATC. This sequence is referred to as the matching portion of the secondary DNA strand displacement primer. This matching portion of a secondary DNA strand displacement primer is complementary to sequences in TS-DNA. The matching portion of a secondary DNA strand displacement primer may be complementary to any sequence in TS-DNA. However, it is preferred that it not be complementary TS-DNA sequence matching either the rolling circle amplification primer or a tertiary DNA strand displacement primer, if one is being used. This prevents hybridization of the primers to each other. The matching portion of a secondary DNA strand displacement primer may be complementary to all or a portion of the target sequence. In this case, it is preferred that the 3′ end nucleotides of the secondary DNA strand displacement primer are complementary to the gap sequence in the target sequence. It is most preferred that nucleotide at the 3′ end of the secondary DNA strand displacement primer falls complementary to the last nucleotide in the gap sequence of the target sequence, that is, the 5′ nucleotide in the gap sequence of the target sequence. The matching portion of a secondary DNA strand displacement primer can be any length that supports specific and stable hybridization between the primer and its complement. Generally this is 12 to 35 nucleotides long, but is preferably 18 to 25 nucleotides long.

It is preferred that secondary DNA strand displacement primers also contain additional sequence at their 5′ end that does not match any part of the OCP or ATC. This sequence is referred to as the non-matching portion of the secondary DNA strand displacement primer. The non-matching portion of the secondary DNA strand displacement primer, if present, serves to facilitate strand displacement during DNA replication. The non-matching portion of a secondary DNA strand displacement primer may be any length, but is generally 1 to 100 nucleotides long, and preferably 4 to 8 nucleotides long.

Another form of DNA strand displacement primer, referred to herein as a tertiary DNA strand displacement primer, is an oligonucleotide having sequence complementary to part of the sequence of an OCP or ATC. This sequence is referred to as the complementary portion of the tertiary DNA strand displacement primer. This complementary portion of the tertiary DNA strand displacement primer matches sequences in TS-DNA. The complementary portion of a tertiary DNA strand displacement primer may be complementary to any sequence in the OCP or ATC. However, it is preferred that it not be complementary OCP or ATC sequence matching the secondary DNA strand displacement primer. This prevents hybridization of the primers to each other. Preferably, the complementary portion of the tertiary DNA strand displacement primer has sequence complementary to a portion of the spacer portion of an OCP. The complementary portion of a tertiary DNA strand displacement primer can be any length that supports specific and stable hybridization between the primer and its complement. Generally this is 12 to 35 nucleotides long, but is preferably 18 to 25 nucleotides long. It is preferred that tertiary DNA strand displacement primers also contain additional sequence at their 5′ end that is not complementary to any part of the OCP or ATC. This sequence is referred to as the non-complementary portion of the tertiary DNA strand displacement primer. The non-complementary portion of the tertiary DNA strand displacement primer, if present, serves to facilitate strand displacement during DNA replication. The non-complementary portion of a tertiary DNA strand displacement primer may be any length, but is generally 1 to 100 nucleotides long, and preferably 4 to 8 nucleotides long. A rolling circle amplification primer is a preferred form of tertiary DNA strand displacement primer.

DNA strand displacement primers may also include modified nucleotides to make them resistant to exonuclease digestion. For example, the primer can have three or four phosphorothioate linkages between nucleotides at the 5′ end of the primer. Such nuclease resistant primers allow selective degradation of excess unligated OCP and gap oligonucleotides that might otherwise interfere with hybridization of detection probes, address probes, and secondary OCPs to the amplified nucleic acid. DNA strand displacement primers can be used for secondary DNA strand displacement and strand displacement cascade amplification, both described below.

Peptide nucleic acids (PNA) are a modified form of nucleic acid having a peptide backbone. Peptide nucleic acids form extremely stable hybrids with DNA (Hanvey et al., Science 258:1481-1485 (1992); Nielsen et al., Anticancer Drug Des. 8:53-63 (1993)), and have been used as specific blockers of PCR reactions (Orum et al., Nucleic Acids Res., 21:5332-5336 (1993)). PNA clamps are peptide nucleic acids complementary to sequences in both the left target probe portion and right target probe portion of an OCP, but not to the sequence of any gap oligonucleotides or filled gap space in the ligated OCP. Thus, a PNA clamp can hybridize only to the ligated junction of OCPs that have been illegitimately ligated, that is, ligated in a non-target-directed manner. The PNA clamp can be any length that supports specific and stable hybridization between the clamp and its complement. Generally this is 7 to 12 nucleotides long, but is preferably 8 to 10 nucleotides long. PNA clamps can be used to reduce background signals in rolling circle amplifications by preventing replication of illegitimately ligated OCPs.

Open circle probes, gap oligonucleotides, rolling circle amplification primers, detection probes, address probes, amplification target circles, DNA strand displacement primers, and any other oligonucleotides can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

In certain embodiments, the invention provides methods for ligating the free ends of a target nucleic acid. For example, a target nucleic acid construct with free ligatable ends may be ligated intramolecularly to produce a closed circular vector. Alternatively, a target nucleic acid with free ligatable ends may be ligated intermolecularly to a vector with free compatible ends. Any DNA ligase is suitable for use in the disclosed amplification method. Preferred ligases are those that preferentially form phosphodiester bonds at nicks in double-stranded DNA. That is, ligases that fail to ligate the free ends of single-stranded DNA at a significant rate are preferred. Thermostable ligases are especially preferred. Many suitable ligases are known, such as T4 DNA ligase (Davis et al., Advanced Bacterial Genetics-A Manual for Genetic Engineering (Cold Spring Harbor Laboratory, Cold Spring Harbor, N.Y., 1980)), E. coli DNA ligase (Panasnko et al., J. Biol. Chem. 253:4590-4592 (1978)), AMPLIGASE [r] (Kalin et al., Mutat. Res., 283(2):119-123 (1992); Winn-Deen et al., Mol Cell Probes (England) 7(3):179-186 (1993)), Taq DNA ligase (Barany, Proc. Natl. Acad. Sci. USA 88:189-193 (1991), Thermus thermophilus DNA ligase (Abbott Laboratories), Thermus scotoductus DNA ligase and Rhodothermus marinus DNA ligase Thorbjarnardottir et al., Gene 151:177-180 (1995)). T4 DNA ligase is referred for ligations involving RNA target sequences due to its ability to ligate DNA ends involved in DNA:RNA hybrids (Hsuih et al., Quantitative detection of HCV RNA using novel ligation-dependent polymerase chain reaction, American Association for the Study of Liver Diseases (Chicago, Ill., Nov. 3-7, 1995)).

4.12. Hybrid Capture Applications

As described above, the invention provides for methods of switching or flipping a target nucleic acid library from one vector system to another. The invention further provides methods for altering a target nucleic acid library to, for example, convert a partial cDNA library, such as a secretion trap library, to a library of full-length cDNA target nucleic acid molecules. This method of conversion may also be used, optionally, to also effect switching or flipping of the library from one vector system to another. For example, a mammalian secretion trap library in a first vector system can be converted to a full-length cDNA library in a second vector system by first excising the partial cDNA target nucleic acid sequences of the secretion trap library by a recombinase or traditional restriction and ligation cloning techniques. A circularized sequence containing the partial target nucleic acid sequence is then used to generate a single-stranded hybrid capture probe using rolling circle amplification and, for example, a Phi 29 DNA polymerase. The hybrid capture probe population is then used to select a corresponding full-length cDNA probe by, for example, hybridization to a single-stranded full-length or denatured double-stranded full-length cDNA library (hybrid selection). The partial cDNA target nucleic acid sequences present on the hybrid capture probe will select out complementary full-length cDNA sequences present in the second library. The second library may utilize the same expression vector as was used in the first expression library—in which case a partial cDNA library is simply converted to a corresponding full-length cDNA library based upon the same vector. Alternatively, the second library may utilize a different expression library from the first—in which case a partial cDNA library based upon a first expression vector is converted into a full-length expression vector based upon a second expression vector. Methods for effecting these library switching/flipping or conversion to full-length expression systems will be apparent to the skilled artisan in light of the foregoing disclosures. Additional library construction and manipulation methods are described below.

Methods of synthesis of target nucleic acid libraries, for example target nucleic acid cDNA libraries, are well known in the art. Purification of mRNA is a technique well known to those of skill in the art. The exact methods used for purification depend on the organism or cell type from which the mRNA is to be obtained. Briefly, the cells are lysed, DNA is digested with RNase free DNase, total RNA is either ethanol precipitated or banded in CsCl, and mRNA is purified from total RNA by chromatography on an oligo(dT)-cellulose or poly(U)-Sepharose column (Berger, S. L., Methods in Enzymology 152:215-219 (1987); MacDonald, R. J. et al., Methods in Enzymology 152:219-227 (1987); Jacobson, A., Methods in Enzymology 152:254-261 (1987)). One should prepare mRNA using a method most suitable for the cell type they are using.

Purified poly A(+) mRNA is precipitated and redissolved at 250 ng/μl in 5 mM HEPES pH 7.5, 0.1 mM EDTA. This RNA is denatured by mixing 8.0 μl (2.0 μg) with 0.9 μl 100 mM methyl mercury hydroxide and warming at 65° C. for 5 minutes. This is neutralized by addition of 0.9 μl 350 mM beta-mercaptoethanol. The resulting solution is vortexed and centrifuged briefly in a microfuge to spin down the drops. Neutralization is allowed to occur for at least 1 minute. The first strand synthesis reaction is prepared by mixing 8.0 μl of reverse transcription primer (the primer must not be phosphorylated at the 5′ end), 8.2 μl H₂O, 8.0 μl 5×BRL RT buffer (reverse transcription buffer from Bethesda Research Laboratories), 2.0 μl 100 mM dithiothreitol, 1.6 μl 10 mM dNTP mix, and 0.4 μl RNasin. This is warmed to 37° C. and then Superscript II reverse transcriptase is added at 1.0 μl per μg RNA. This is warmed at 37° C. for 60 minutes. The reaction is stopped by addition of 1.0 μl 500 mM EDTA and warming at 65° C. for 10 minutes. The amount of reverse transcriptase primer used in this reaction will vary depending on the specific primer. Final primer concentrations in the range of 1-30 μM are normally used. The expected yield is 0.2-0.4 μg of first strand per μg of input mRNA when a random primer is used and 0.5-1.0 μg when an oligo dT primer is used.

The first strand synthesis reaction is fractionated on a Sepharose CL-4B column (Eschenfeldt, W. H. and Berger, S. L., Methods in Enzymology 152:335-337 (1987)). This separates high from low molecular weight material and will remove unincorporated dNTPs, excess primers and other low molecular weight material. The high molecular weight fractions are pooled. If the column is run using a buffer of 10 mM Tris pH 7.4, 10 mM KCl, 0.1 mM EDTA the pooled fractions can be used directly in the following step. Otherwise, ethanol precipitate the nucleic acid and resuspend in the said buffer.

Mix 1 μg heteroduplex cDNA produced in the first strand synthesis, 40 μl 10×1 for-all buffer (this is 250 mM Tris acetate pH 7.7, 500 mM potassium acetate, 100 mM magnesium acetate), 4.0 μl 1M ammonium sulfate, 20.0 μl 100 mM dithiothreitol, 10.0 μl 2 mM dNTP mix (prepare a dNTP mix at about 1.0 Curie/millimole in ³²P dATP which is 1.5×10⁶ disintegrations per minute (dpm) per microgram of second strand or 0.82×10⁶ dpm/μg of ds cDNA), 4.0 μl 15 mM NAD (nicotine adenine dinucleotide), 4 μl 10 mg/ml bovine serum albumin, 1.6 μl RNase H at 1unit/μl, 2.0 μl E. coli ligase at 2units/μl, DNA polymerase I at 10units/μl , and H₂O to give final volume of 400 μl. Incubate this at 14° C. for 8-16 hours. Stop the reaction by addition of 10 μl 500 mM EDTA. The ds cDNA is then purified. Any type of purification step can be used, a wide variety of columns for such purposes being commercially available. The Qiaquick PCR cleanup procedure has been found to work well. This column is eluted with approximately 501 μl of 10 mM Tris pH 8.5, 1.0 mM EDTA. This second strand synthesis step converts the heteroduplex cDNA quantitatively to ds cDNA.

In practice the step of second strand synthesis leaves many cDNA molecules with ragged ends, i.e., they are not fully double stranded at both ends but may have an overhang of single stranded region. This polishing step removes any single stranded ends of the cDNA. Polishing is accomplished by mixing 2.0 μg ds cDNA, 10.0 μl 10×-1-for-all buffer, 1.0 μl 100 mM dithiothreitol, 3.0 μl 10 mM dNTP mix (yielding 300 μM of each dNTP), 1.4 μl 1.5 mM NAD, 4.0 μl RNase A at 1 ng/μl, 1.6 μl RNase H at 1unit/μl, 4.0 μl E. coli ligase at 2units/μl, 1.65 μl T4 DN polymerase at 4units/μl, 1.34 μl T7 DNA polymerase at 10units/μl, and bring to 100 μl volume with H₂O. Incubate at 15° C. for 15 minutes then stop the reaction by addition of 3.0 μl 500 mM EDTA. Phenol:chloroform extract the mix and back extract with ammonium acetate:isopropanol with 1 μg of glycogen carrier. Rinse with 80% ethanol, dry in a lyophilizer, and resuspend the DNA in 10 μl of TE.

In certain embodiments, an adaptor of known sequence may be ligated to the end of the cDNA. This adaptor is used to design two nested primers to be used in the two rounds of PCR which are performed in later steps of the invention. The adaptor consists of two complementary strands of DNA. The “top” strand of the adaptor, which corresponds to the mRNA strand, is synthesized such that it is a normal strand of DNA with a 3′ OH group. The “lower” strand of the adaptor, corresponding to the first strand of cDNA synthesized, is made such that it has a 5′ OH group and a 3′ NH₂ group. The adaptor is added as follows: the two strands of the adaptor are added to a solution such that the final concentration of each is 25 μM. Add to this 2.0 μl 10×1 for-all buffer and bring to a total of 20 μl 1 with H₂O. Incubate at 90° C. for 30 seconds, 65° C. for 5 minutes, then leave at room temperature for 5 minutes. This allows for hybridization of the two strands of adaptor to each other.

The ligation reaction consists of mixing 10.0 μl of the cDNA preparation from above, 6.0 μl of the hybridized adaptors, 2.4 μl 10×1 for-all buffer, 3.0 μl 10 mM hexamine cobalt (III) chloride, 3.0 μl of a 0.5 mM ATP, 50 mM dithiothreitol mix, 0.75 μl of T4 DNA ligase at 400units/μl (New England Biolabs), and adjusting to 30 μl total volume with H₂O. Incubate at 14° C. for 18 hours. Add EDTA and heat kill the reaction at 65° C. for 10 minutes. Clean-up of the ligated cDNA.

Fractionate the ligated cDNA on a Sepharose CL-4B column to remove excess primers and other low molecular weight impurities. Pool the high molecular weight fractions. If the column is run using 10 mM Tris pH 8.4, 10 mM KCl, 0.1 mM EDTA the pooled fractions can be used directly in the following PCR without precipitation.

Perform a standard polymerase chain reaction on the cDNA with the attached adaptor. Polymerase chain reaction protocols are well known to those of skill in the art. See M. A. Innis et al., PCR Protocols: A Guide to Methods and Applications (Academic Press, Inc., New York (1990)). If several different cDNA preparations with attached adaptors were prepared, the one with the greatest abundance of the desired gene is chosen. The abundance of cDNA in the various preparations can be tested by performing limiting dilution analysis PCR using two gene specific primers. Since the object is to recover the 5′ end of the gene, these two primers should be near the 5′ end of the already known sequence, roughly 100 base pairs apart from each other. Limiting dilution analysis PCR is simply performing PCR on a series of dilutions of a sample and determining how much the sample can be diluted and still show a discrete amplified band of DNA when the products are run on a gel. Once the best cDNA sample has been chosen perform PCR as follows: as one primer use a gene specific oligomer located approximately 100-200 bases from the 5′ end of the already known sequence of the cDNA. The second primer is complementary to the 5′ region of the ligated adaptor. It is preferable to use a hot start PCR. The preferred conditions are to use TaqPlus DNA polymerase with 1×C-PCR buffer (20.0 mM TrisHCI pH 9.0, 8.5 mM NaCl, 10.0 mM KCl, 10.0 mM (NH₄)₂SO₄, 2.0 mM MgSO₄, 0.1% Triton X-100). PCR conditions are 95° C. for 30 seconds followed by cycles of 96° C. for 4 seconds, 65°-70° C. (depending on the primers) for 10 seconds, and 72° C. The 72° C. time depends on the length of the expected product, leaving this extension reaction at 72° C. for 1 minute per kilobase of length of the longest expected product.

It is useful to set up the PCR as a cycle titration, using 0.2-2.0 ng of cDNA as the substrate for each reaction. Trace label these by making the reaction solutions approximately 0.1 Curie/millimole in dATP so that quantification can be performed at later steps. Several PCRs are performed to titrate between 18 to 30 cycles. Run each of the titrations on an agarose gel and choose the number of cycles which gives an even, nonsaturated smear as the substrate for the next step. The reactions will often show bands, but these are likely to be spurious unless the transcript was quite abundant. The amplified product from the best titration cycle is then purified. This can be done by gel purifying on an agarose gel, recovering DNA from the region of 500-5000 base pairs, or by chromatography on a CL-4B or CL-6B column. The DNA is ethanol precipitated, dried, and resuspended in TE. The DNA concentration can be determined from the specific activity of the DNA.

Gene specific enrichment is calculated at this stage by limiting dilution analysis PCR. Use two gene specific oligomer primers for this. This result is compared with the value determined from the limiting dilution analysis PCR previously performed on the initially synthesized cDNA solution to determine whether enrichment has occurred.

The amplified DNA is enriched in the product of interest, but in practice it will be only a small percentage, quite possibly only a fraction of 1%, of the amplified material. This is especially true if the specific mRNA was of low abundance. The method of the invention allows for hybrid selection using a hybrid capture probe generated by rolling circle amplification of a first library (e.g. a partial cDNA secretion library) to select corresponding full-length cDNAs from a second library (e.g. a full-length cDNA library in a mammalian retroviral vector). A hybrid capture step can result in a fairly dramatic enrichment for the cDNA of interest, enrichments of at least 10-100 fold commonly being seen and enrichments of 100-1000 fold sometimes being seen. Hybrid capture is performed by preparing a biotin labeled gene specific oligomer (B-GSO), hybridizing this in solution with the amplified cDNA which has been denatured, and capturing the cDNA bound to the B-GSO with a magnet. This is a known technique, wherein the biotin binds to streptavidin which is attached to paramagnetic particles and it is these which are attracted to the magnet while holding the biotin which in turn is attached to the gene specific oligomer which is hybridized to the cDNA of interest. Unbound molecules can be washed away. The only amplified cDNA which is captured is that which hybridizes with the biotin labeled gene specific oligomer. Other spuriously amplified DNA or DNA which by chance was complementary to GSP#R1 (which had been used as the specific primer for PCR) but is not complementary to B-GSO will be washed away. The specific steps for hybrid capture are as follow: mix 5-50 ng of the primary amplified cDNA into a solution consisting of final concentrations of 10 mM sodium phosphate pH 7.0, 1 mM EDTA, 2.4M tetraethyl ammonium chloride, and 0.1 nM of the biotin labeled gene specific oligomer. This is conveniently done by first preparing a 50×capture mix consisting of 79.0 μl H₂O, 1.0 μl B-GSO at 1 μM, 100.0 μl 1M sodium phosphate pH 7.0 and 20.0 μl 500 mM EDTA. Mix 2.0 μl of this 50×capture mix with 18.0 μl amplified cDNA containing 5-50 ng of the cDNA and 80.0 μl 3 M tetraethyl ammonium chloride (Sigma). Incubate this at 90° C. for 5 minutes followed by 25° C. (or room temperature) for 60 minutes. To this add 10.0 μl washed streptavidin paramagnetic particles (Dyna) (the beads are washed in a solution (bead wash and stringency wash solution) consisting of 1×capture mix, 2.4M tetraethyl ammonium chloride and diluted 5 fold in this same solution). Leave at 25° C. (room temperature) for 60 minutes with occasional stirring. Capture the beads with a magnet. Add 100.0 μl of the stringency wash solution to resuspend the beads and incubate at 35° C. for 10 minutes. Recapture with a magnet. Wash the captured beads twice with 100.0 μl of 10.0 mM Tris pH 8.7, 50 mM KCl, 0.1 mM EDTA at room temperature. Resuspend the beads in 15.0 μl of 5 mM Tris pH 8.7, 0.1 mM EDTA.

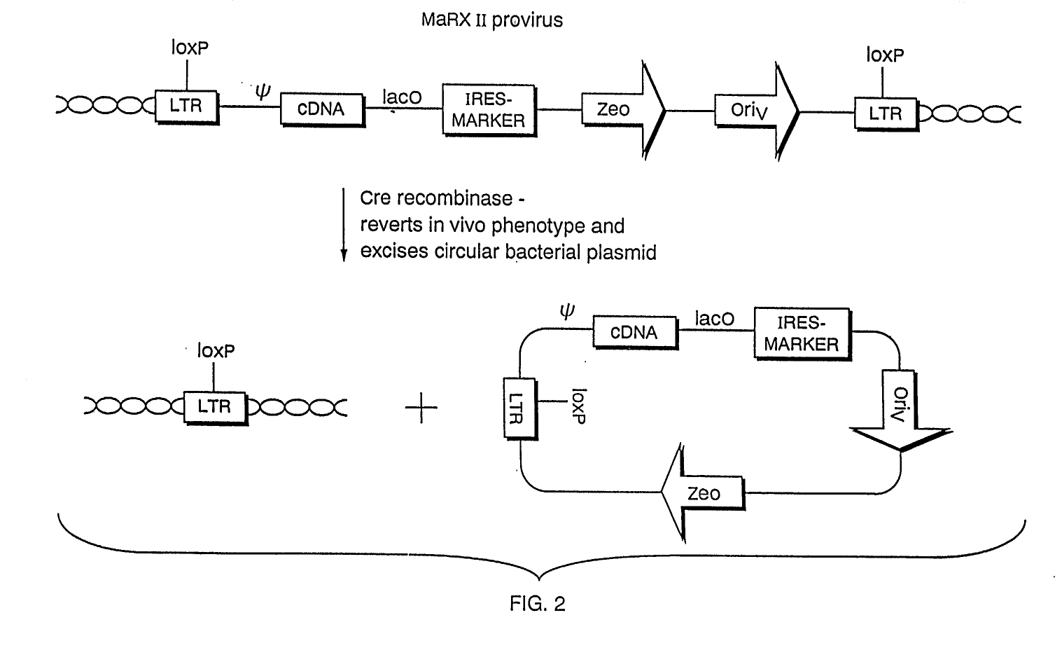

A second round of PCR is performed using the hybrid captured cDNA as the template. A pair of nested primers, internal to the primers used for the first round of PCR, is to be used. The 3′ primer is a gene specific primer, shown as GSP#R2 in FIG. 2. The 5′ primer, called Anchor 2B as shown in FIG. 2, is complementary to the adaptor, but will be internal (3′) to Anchor 2A. PCR is then carried out as with the first round of PCR. Again, it is preferred to use hot start PCR, to do a cycle titration of 18-30 cycles, trace label the reactions by including approximately 0.1 Curie/millimole dATP, and check the reaction products on a gel. This round of PCR is likely to yield discrete bands and these correspond to the gene of interest. This amplified material can be gel purified and cloned or sequenced directly. If material is limiting it can simply be reamplified. If a smear is seen instead of a discrete band, choose the number of cycles which gives an even, nonsaturated smear and run on an agarose gel to purify size fractions or chromatograph on a CL-4B or CL-6B column and clone and sequence the products.

All of the preceding examples as well as the discussion have described the invention as being solely directed to use with cDNA. The invention is quite adaptable to use with other nucleic acid such as with genomic DNA. For this method, one purifies genomic DNA, denature the DNA, dephosphorylate the genomic DNA, prepare a gene specific oligomer primer, mix the dephosphorylated genomic DNA and single primer, and allow synthesis of a single strand as an extension of the primer. This is done by addition of the 4 dNTPs, DNA polymerase, buffers, etc. by methods well known to those skilled in the art. Following this first strand synthesis, an adaptor is ligated to the 3′ end. The adaptor is designed to have a 3′ amino group to prevent it from ligating to the 5′ end of any free primer or newly synthesized DNA. The anchor may ligate to 3′ ends of genomic DNA, but such pieces of DNA will amplify in only a linear fashion during the following PCR and will result in only minor “impurities”. A PCR is then performed using a gene specific oligomer as one primer and an oligomer complementary to the adaptor as the second primer.

The disclosed invention illustrates a new technique to purify and analyze rapidly the ends of genes. It obviates the need for library screening. It can be biased to result in purification of either a 5′ end or a 3′ end of a gene. The method can be performed using cDNA or genomic DNA. The technique uses 3 steps of enrichment-two separate rounds of PCR and a hybrid capture step. These steps result in production of a product which may be so enriched in the desired product that the final DNA produced may be sequenced without cloning. In some instances it may be necessary to clone the product, but it is necessary to characterize only a few clones to find one of interest. This can easily be done by preparing DNA minipreps and sequencing this miniprep DNA. The method is rapid and by eliminating the necessity of library screening is much less labor intensive than earlier methods of finding and studying genes.

Many modifications of this procedure will be apparent to those of skill in the art. Only a few of the possibilities have been described herein. Obvious modifications which have not been presented are considered to be equivalent to the disclosed methods.

### EXAMPLES

### Example 1

### Construction of the Retroviral MaRX II Vector

The following example provides the methods for the construction of replication-defective retrovirus, pMaRX II. The starting vector is pBABE-puro (Morgenstern, 1990, Nucleic Acids Res. 18: 3587-3596), which is modified as follows:

The insertion of a synthetic linker comprising a loxP site was into the NheI site. The sequence of the linker containing the loxP site is as follows:

| | |
|---|---|
| 5′CTAGCATAACTTCGTATAATGTATGCTATACGAAG | (SEQ ID No.1) |
| TTATGTATTGAAGCATATTACATACGATATGCTTCAA | |
| TAGATC-3′. | |

The insertion of this synthetic linker creates a loxP site while simultaneously destroying the 3′ NheI site, leaving a unique NheI site.

The insertion of a polylinker between the BamHI and SalI sites of pBABE-puro which contains a primer binding site for the universal (−20) sequencing primer and the lac operator sequence (lacO). The sequence of the upper strand of the polylinker is as follows:

| | |
|---|---|
| 5′GGATCCGTAAAACGACGGCCAGTTTAATTAAGAAT | (SEQ ID No.2) |
| TCGTTAACGCATGCCTCGAGTGTGGAATTGTGAGCGG | |
| ATAACAATTTGTCGAC-3′. | |

The insertion of a PCR fragment comprised of the bacterial EM7 promoter and the zeocin resistance gene was amplified from pZEO SV (Invitrogen) such that the SalI and StuI sites were included at the 5′ end of the fragment and the BspEI and ClaI sites were included at the 3′ end of the fragment. The modified pBABE-puro vector was digested with SalI and ClaI and ligated with the PCR fragment. The sequence of the upper strand of the PCR fragment is as follows:

| | |
|---|---|
| 5′gtcgacaggcctCGGACCTGCAGCACGTGTTGACA | (SEQ ID No.3) |
| ATTAATCATCGGCATAGTATATCGGCATAGTATAATA | |
| CGACTCACTATAGGAGGGCCACCATGGCCAAGTTGAC | |
| CAGTGCCGTTCCGGTGCTCACCGCGCGCGACGTCGCC | |
| GGAGCGGTCGAGTTCTGGACCGACCGGCTCGGGTTCT | |
| CCCGGGACTTCGTGGAGGACGACTTCGCCGGTGTGGT | |
| CCGGGACGACGTGACCCTGTTCATCAGCGCGGTCCAG | |
| GACCAGGTGGTGCCGGACAACACCCTGGCCTGGGTGT | |
| GGGTGCGCGGCCTGGACGAGCTGTACGCCGAGTGGTC | |
| GGAGGTCGTGTCCACGAACTTCCGGGACGCCTCCGGG | |
| CCGGCCATGACCGAGATCGGCGAGCAGCCGTGGGGGC | |
| GGGAGTTCGCCCTGCGCGACCCGGCCGGCAACTGCGT | |
| GCACTTCGTGGCCGAGGAGCAGGACTGAttccggatt | |
| tatcgat-3′. | |

The insertion of a PCR fragment comprised of the RK2 Oriᵥ which was amplified from the plasmid pMYC3 (Shah et al., 1995, J. Mol. Biol. 254: 608-622). The minimal Oriᵥ was chosen as defined in Shah et al. This PCR fragment contained a BspEI site at its 5′ end and BglII and ClaI sites at its 3′ end. The modified pBABE-puro vector and the PCR fragment were both digested with BspEI and ClaI and ligated together. The sequence of the top strand of the PCR fragment is as follows:

| | |
|---|---|
| 5′TCCGGAcgagtttcccacagatgatgtggacaagc | (SEQ ID No.4) |
| ctggggataagtgccctgcggtattgacacttgaggg | |
| gcgcgactactgacagatgaggggcgcgatccttgac | |
| acttgaggggcagagtgatgacagatgaggggcgcac | |
| ctattgacatttgaggggctgtccacaggcagaaaat | |
| ccagcatttgcaagggtttccgcccgtttttcggcca | |
| ccgctaacctgtcttttaacctgcttttaaccaatat | |
| ttataaaccttgtttttaaccagggctgcgccctggc | |
| gcgtgaccgcgcacgccgaaggggggtgccccccctt | |
| ctcgaaccctcccggAGTCTatcgat3′. | |

The inclusion of a pUC origin (Ori_{pUC}) of replication in an equivalent position to the RK2 Oriᵥ in either orientation was found to reduce both viral titer and expression levels in infected cells.

The f1 origin of replication was also inserted in the modified pBABE-puro vector. The f1 origin of replication was amplified from pBluescript SK+ (Stratagene) and NotI restriction sites were added to the 5′ and 3′ ends. This fragment was inserted into the modified vector following digestion of both the modified pBABE-puro vector and the fragment with NotI. An orientation of the f1 origin was chosen that would yield, upon helper rescue, the sense strand of the cDNA. The sequence of the amplified f1 fragment is as follows:

| | |
|---|---|
| 5′gcggccgcGGGACGCGCCCTGTAGCGGCGCATTAA | (SEQ ID No.5) |
| GCGCGGCGGGTGTGGTGGTTACGCGCAGCGTGACCGC | |
| TACACTTGCCAGCGCCCTAGCGCCCGCTCCTTTCGCT | |
| TTCTTCCCTTCCTTTCTCGCCACGTTCGCCGGCTTTC | |
| CCCGTCAAGCTCTAAATCGGGGGCTCCCTTTAGGGTT | |
| CCGATTTAGTGCTTTACGGCACCTCGACCCCAAAAAA | |
| CTTGATTAGGGTGATGGTTCaCGTAGTGGGCCATCGC | |
| CCTGATAGACGGTTTTTCGCCCTTTGACGTTGGAGTC | |
| CACGTTCTTTAATAGTGGACTCTTGTTCCAAACTGGA | |
| ACAACACTCAACCCTATCTCGGTCTATTCTTTTGATT | |
| TATAAGGGATTTTGCCGATTTCGGCCTATTGGTTAAA | |
| AAATGAGCTGATTTAACAAAAATTTAACGCGAATTTT | |
| AACAAAATATTAACGTTTACAAgcggccgc-3′. | |

The vector was further modified by the insertion of a Pacd site between the BglII and ClaI sites of the modified pBABE-puro vector using the following synthetic fragment:

| | |
|---|---|
| 5′-GATCTTTAATTAAAT-3′ | (SEQ ID No.6) |
| 3′-AAATTAATTTAGC-5′ | (SEQ ID No.7) |

The vector was still further modified by the insertion of a PmeI site into the BspEI of the modified pBABE-puro vector site using the following synthetic fragment:

| | |
|---|---|
| 5′-CCGGGTTTAAACT-3′ | (SEQ ID No.8) |
| 3′-CAAATTTGAGGCC-5′ | (SEQ ID No.9) |

The insertion of this fragment destroys one BspEI site, leaving the second site intact.

The vector was further modified by the insertion of a fragment comprising an IRES(EMCV)-Hygromycin resistance marker. The IRES hygromycin resistance cassette was created by amplification of the Hygromycin sequence from pBabe-Hygro (Morgenstern et al., 1990, Nucl. Acids Res. 18: 3587-3596) such that it lacked the first methionine of the hygromycin coding sequence and such that ClaI and SalI sites were added following the stop codon. This was inserted into the IRES-containing vector, pCITE (digested MscI-SalI) such that the first methionine of the hygromycin protein was donated by the vector. Methionine placement is critical for efficient function of the IRES. This cassette was amplified by PCR such that a SalI site was added upstream of the functional IRES and was re-inserted into the pBabe-Hygro following digestion of both with SalI and ClaI. This fragment was excised and inserted into the SalI site of the modified vector such that SalI sites were reformed on both sides.

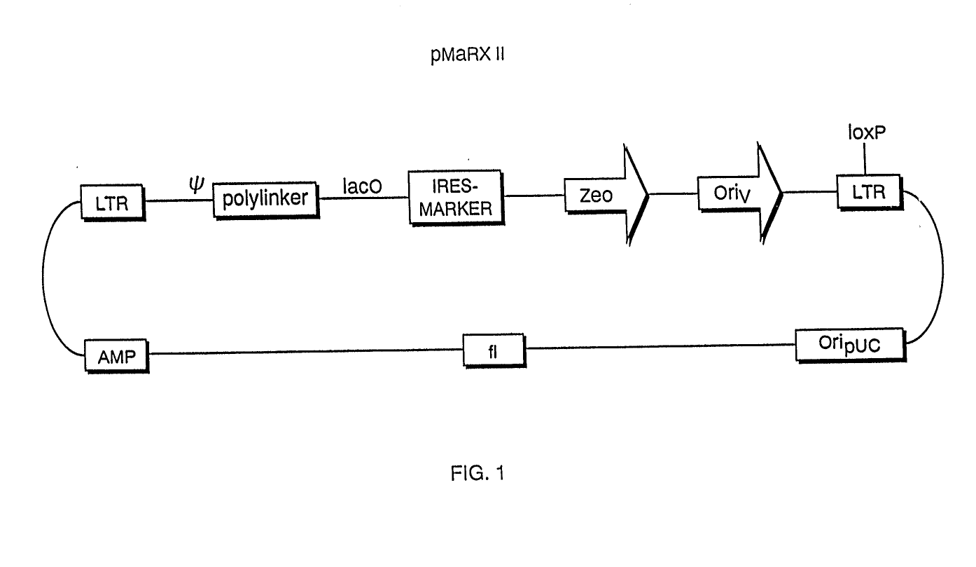

The resulting vector is the MaRX II backbone (FIG. 1). The derivation of the specific purpose vectors from the MaRX II backbone is described below.

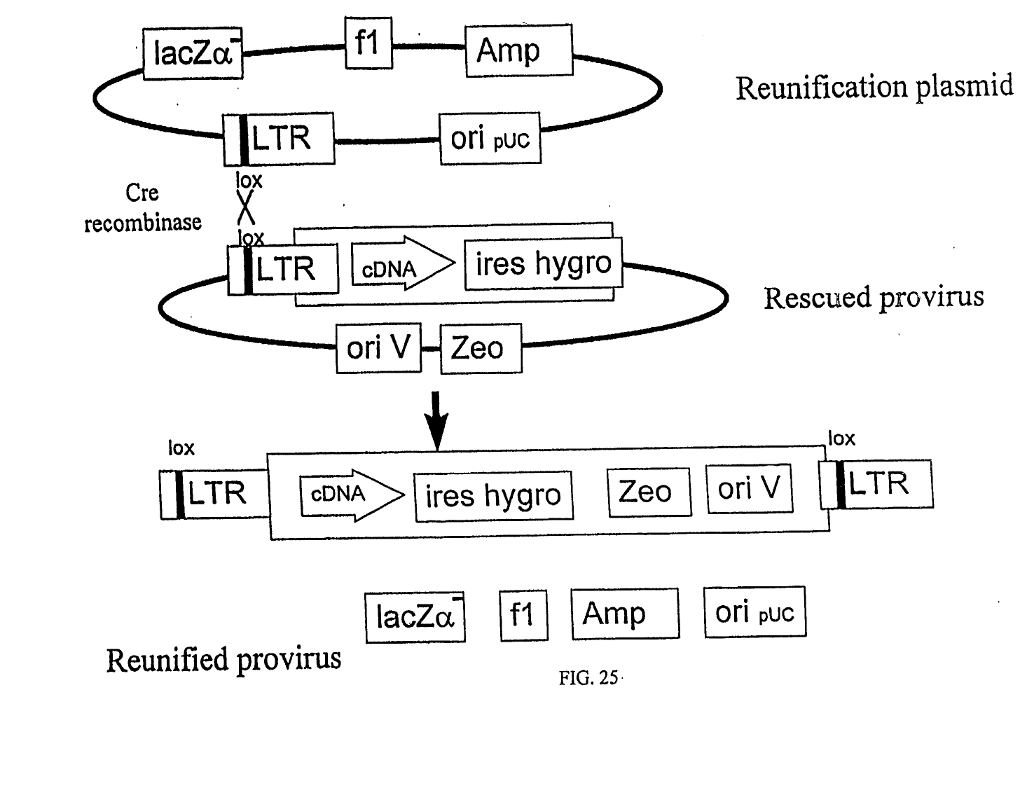

In the illustrated MaRX II vector, excision of the provirus by recombinase treatment or the like, because of the location of the recombinase sites in the LTR sequences, results in a closed, circular vector with only one LTR. In the illustrated embodiment, the defective LTR cannot be used to make virus. However, another aspect of the present invention provides a convenient means for adding back LTR elements necessary for generating an infectious (though still replication-deficient) retroviral vector. As illustrated in FIG. 25, we derived the so-called reunification vector to provide, by recombinase mediated ligation, a vector in which the LTR sequences have been restored and the resulting vector can be used, e.g., upon isolation from bacterial cells in which it may be amplified, in the transient transfection of the packaging cell lines and the generation of a second round of infectious viral particles. In its simplest of embodiments, the subject method provides an second construct (the reunification vector) having an LTR with a recombination site which can bring about cross-ligation of the second construct with the retroviral vector so as to recapitulate a vector which includes the original retroviral sequences now being flanked by LTR sequences at both the 5′ and 3′ ends. The reunification vector may contain other sequence elements in addition to the modified LTR with a recombination site (such as loxP). For example, the reunification plasmid may also contain a bacteria replication origin sequence (such as Ori_{pUC}), an antibiotic resistance gene (such as resistance genes for Ampicilin, Kanamycin, or tetracyclin, etc.), an f1 sequence, an lacZα⁻ sequence, or any other functional elements. These elements need not be in the particular order on the reunification plasmid as shown in FIG. 25, and, unless specifically stated, each particular element may use either of the two possible orientations when appropriate.

### Example 2

### Construction of the Retroviral Vector for Sense Complementation Screening

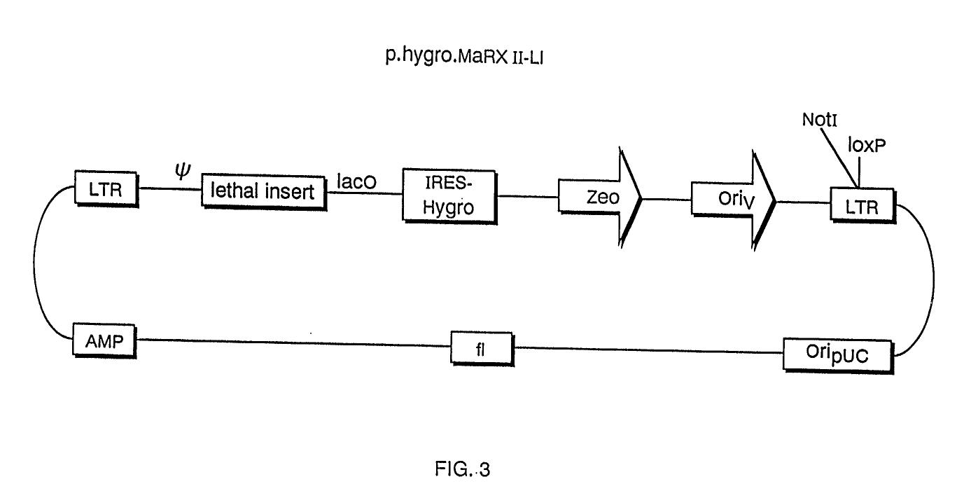

This example provides the methods for constructing the sense-expression complementation screening vector, a pMaRX II derivative vector, pHygro MaRX II-LI (FIG. 3). The starting point for the construction of this vector begins with the MaRX II vector, as described above.

The vector is further modified by the insertion of a synthetic NotI linker which was ligated into the NheI site such that only one NheI site was left intact. The sequence of the NotI linker is as follows:

| | |
|---|---|
| 5′-CTAGATGCGGCCGCTAG-3′ | (SEQ ID No.10) |
| 3′-TACGCCGGCGATCGATC-5′ | (SEQ ID No.11) |

A PCR fragment comprising the SV40 origin (below) was ligated into the PmeI site (in either orientation) to allow for replicative excision. The sequence of the fragment is as follows:

| | |
|---|---|
| 5′GGGGTTTAAACGACTAATTTTTTTTATTTATGCA | (SEQ ID No.12) |
| GAGGCCGAGGCCGCCTCTGCCTCTGAGCTATTCCAG | |
| AAGTAGTGAGGAGGCTTTTTTGGAGGCCCC-3′. | |

The NsiI-NsiI fragment was deleted from pZero (Invitrogen) and this served as a template for the amplification of the lethal insert with primers that recognized the 5′ end of the pTac promoter and the 3′ end of the ccdB coding sequence (the leathal gene). These primers added EcoRI and XhoI sites, respectively. The fragment was inserted following digestion of both the plasmid and the PCR product with EcoRI and XhoI.

This forms the basic sense expression vector. Other markers can replace the IRES-Hygromycin resistance cassette (e.g. IRES-Puromycin resistance, IRES-neomycin resistance, IRES-blasticidin resistance etc.). This vector has been used to produce virus population with titers exceeding 10⁶ particles/ml (as measured on NIH 3T3 cells). This is equivalent to titers obtained from the original pBabe vector. Thus, modifications have not compromised the ability of the vector to produce virus. Furthermore, expression levels obtained from the p.Hygro.MaRX II vectors approximate those obtained with other retroviral vectors (e.g. pBabe). This vector infects with high efficiency a wide variety of tissue culture cells including but not limited to: NIH-3T3, Mv1Lu, IMR-90, WI38, Hep3B, normal human mammary epithelial cells (primary culture), HT1080, HS578t. This vector has been used to test reversion/excision with the result that following infection with a Cre-encoding virus, >99% of cells lose the phenotype conferred by the MaRX II provirus. Following recovery protocols detailed below, >×10³ independent colonies can be routinely recovered from 100 μg of genomic DNA containing the provirus (without T-antigen driven amplification).

### Example 3

### Construction of the Retroviral Vector for Antisense Complementation Screening

This example provides the methods for constructing the antisense screening vectors, the MaRX IIg series, a pMaRX II derivative vector.

Construction of the MaRX IIg series began with a MaRX II vector as described above, except that it lacked the Pacd site. A marker, in most cases hygromycin-resistance, is inserted into the unique SalI site created.

### MaRX IIg

The pMARX II vector was modified by the following steps:

A synthetic polylinker of the following sequence was added between the BamHI and SalI sites of MaRX II.

| | |
|---|---|
| 5′-GATCGTTAATTAACAATTGG-3′ | (SEQ ID No.13) |
| 3′-CAATTAATTGTTAACCAGCT-5′ | (SEQ ID No.14) |

A synthetic NotI linker of the following sequence was ligated into the NheI site such that only one NheI site was left intact.

| | |
|---|---|
| 5′-CTAGATGCGGCCGCTAG-3′ | (SEQ ID No.10) |
| 3′-TACGCCGGCGATCGATC-5′ | (SEQ ID No.11) |

The CMV promoter was inserted into the modified pMARX II vectors as follows. The CMV promoter sequence was amplified from pcDNA3 (Invitrogen) and this served as a template for amplification of the lethal insert with primers using the following oligonucleotides:

| 5′-GGGAGATCTACGGTAAATGGCCCGCC-3′ | (SEQ ID No.15) |
|---|---|
| 5′-CCCATCGATTTAATTAAGTTTAAACGGGCCCTC | (SEQ ID No.16) |
| TAGGCTCGAG-3′ | |

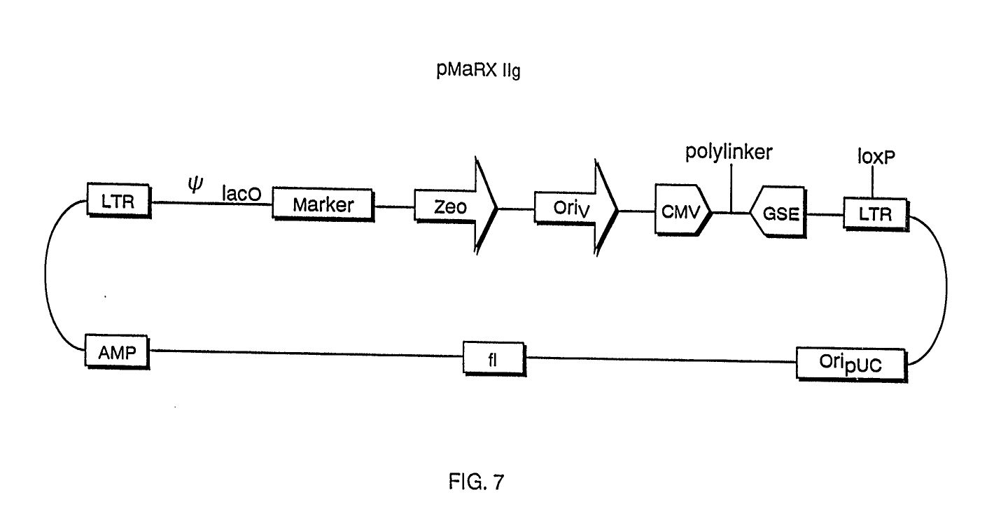

The amplification product was digested with BglII and ClaI and inserted into a similarly digested MaRX II derivative. The polylinker was then altered by the insertion of the EcoRI-XhoI fragment of the MaRX II polylinker between the EcoRI and XhoI sites of the modified vector. This formed the MaRX IIg vector where the CMV promoter drives GSE expression using the 3′LTR polyadenylation signal to terminate the transcript (FIG. 7).

### MaRX IIg-dcmv

The MaRX II derivative from above was digested with NheI. A CMV promoter fragment was prepared by amplification of pHM.3-CMV with the following oligonucleotides:

| 5′-GGGGCTAGCACGGTAAATGGCCCGCC-′3 | (SEQ ID No.17) |
|---|---|
| 5′-CCCTCTAGATTAATTAAGTTTAAACGGGCCCTC | (SEQ ID No.18) |
| TAGGCTCGAG-3′ | |

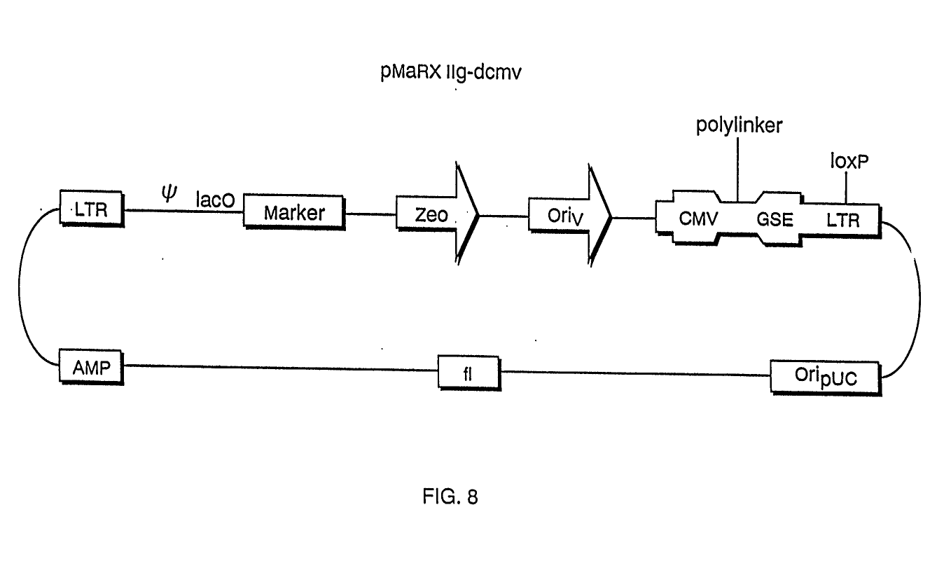

The CMV fragment was digested with NheI and XbaI and ligated to the MaRX II derivative. An orientation was chosen such that transcription proceeded in the same direction as does transcription from the LTR promoter (FIG. 8).

### MaRX IIg-VA

The MaRX II derivative from above (MaRX IIg section) was digested with NheI. An adenovirus VA RNA cassette was prepared by amplification of a modified VA RNA gene (see Gunnery, 1995 Mol Cell Biol 15, 3597-3607 (1995)) with the following oligonucleotides:

| A.5′-GGGGCTAGCCTAGGACCGTGCAAAATGAGAG | (SEQ ID No.19) |
|---|---|
| CC-3′ | |
| B.5′-GGGTCTAGATTAATTAAGTTTAAACGGCCAA | (SEQ ID No.20) |
| AAAAGCTTGCGC-3′ | |

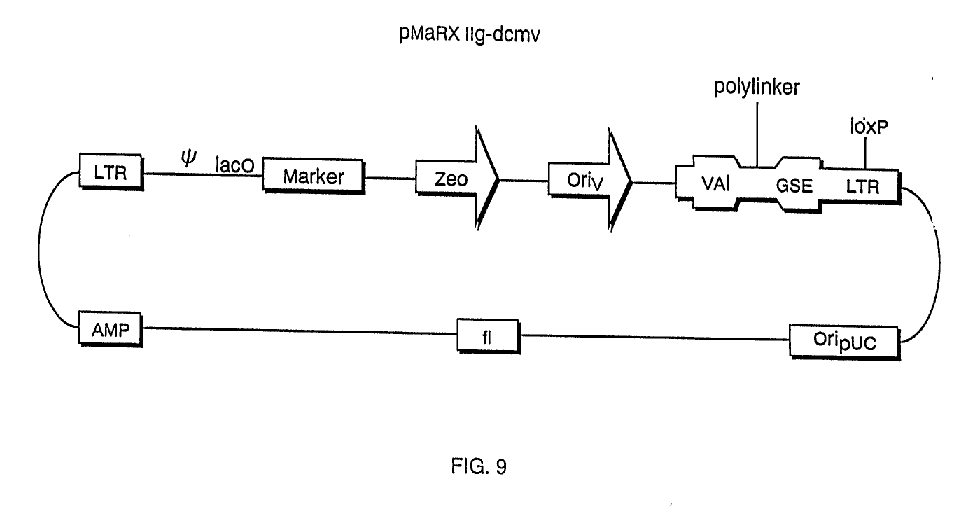

This fragment was digested with NheI and XbaI and ligated into the digested MaRX II derivative. An orientation was chosen such that transcription proceeded in the same direction as does transcription from the LTR promoter (FIG. 9).

All three types of antisense vectors have been used to generate high-titer retroviruses which perform equivalently to p.hygro.MaRX II.

### Example 4

### Construction of the retroviral Vector for Gene Trapping

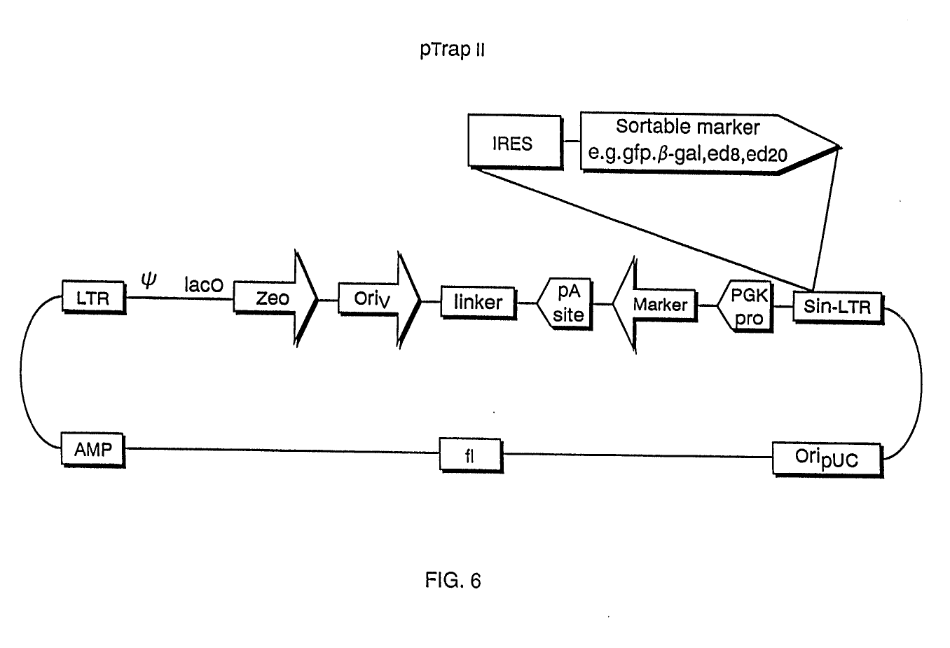

This example provides the methods for the construction of the gene trapping vectors—pTRAP II, a pMaRX II derivative vector (FIG. 6).

The pTRAP II vectors are prepared in a MaRX II backbone, as described above.

The pMaRX II vector was modified by the following steps:

A synthetic polylinker was added between the BamHI and SalI sites of MaRX II, of the following sequence:

| | |
|---|---|
| 5′-GATCGTTAATTAACAATTGG-3′ | (SEQ ID No.13) |
| 3′-CAATTAATTGTTAACCAGCT-5′ | (SEQ ID No.14) |

A second synthetic polylinker was added between the BglII and ClaI sites. The top strand of this linker is as follows:

| | |
|---|---|
| 5′agatctTGTGGAATTGTGAGCGGATAACAATTTG | (SEQ ID No.21) |
| GATCCGTAAAACGACGGCCAGTTTAATTAAGAATTC | |
| GTTAACGCATGCCTCGAGGTCGACatCgat-3′ | |

This incorporates restriction sites for excision from the genome as well as sequencing primer binding sites and the lacO recovery element.

The 3′ LTR and accompanying sequences were removed from the pBabe-puro using ClaI and NotI. These were inserted into a ClaI and NotI digested pBluescript SK+. Site directed mutagenesis was used to delete a segment of the 3′ LTR. This was accompanied by a small insertion. The sequences that surround and thus define the deletion are as follows:

| 5′TAACTGAGAATAGAGAAGTTCAGATCAAGGTCAG | (SEQ ID No.22) |
|---|---|
| GAGATCCCTGAGCCCACAACCCCTCACTCGGGGCGC | |
| -3′ | |

This fragment was re-inserted into ClaI-NotI digested pBabe-puro to create pBabe-puroSIN. This plasmid was the source for the self-inactivating LTR that was inserted into the gene trapping vector using the unique NheI and SapI restriction sites.

The plasmid pPNT (see Brugarolas et al., 1995) was modified by replacement of the neomycin coding sequence with that of hygromycin (from pBabe-Hygro). This created a hygromycin resistance gene flanked by the PGK promoter and the PGK polyadenylation signals. This cassette was amplified by PCR and inserted into the ClaI site of the gene trapping vector such that transcription from the PGK promoter opposed transcription from the 5′ LTR.

A gene trapping cassette was inserted in the NheI site in the 3′ LTR. This gene trapping cassette consists of a quantifiable marker whose expression is promoted by an IRES sequence. In most cases the IRES sequence is derived from EMCV although IRES sequences from other sources are equally suitable. Thus far, IRES linked beta-galactosidase and IRES linked green fluorescent protein markers have been incorporated.

### Example 5

### Construction of the Retroviral Vector for Multiple Organism Display Vector

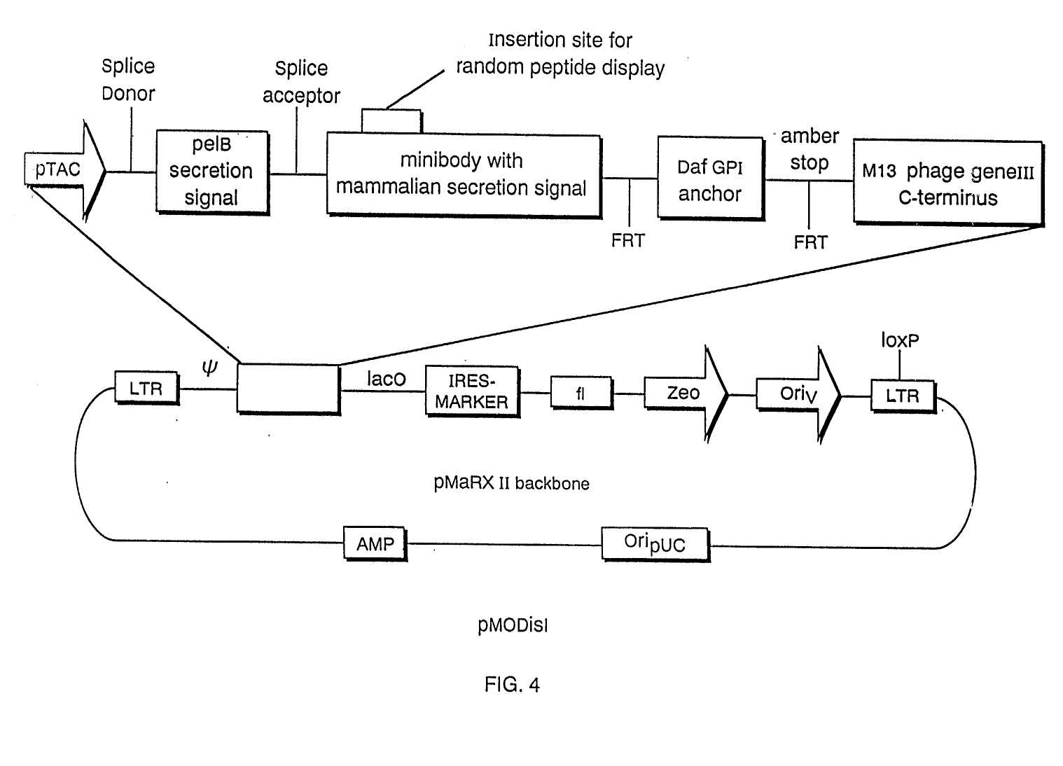

This example provides the methods for constructing the Multiple Organism Display or peptide display vectors—pMODis I and pMODis II, pMaRX II derivative vectors (FIGS. 4 & 5).

The pMODis vectors are designed to act as dual purpose vectors that allow the combination of phage display approaches with functional screening in mammalian systems. These are designed to allow the display of random peptide segments on the surface of filamentous bacteriophage. The displayed peptides can be screened via an affinity approach with a known ligand or a complex mixture of ligands (e.g. fixed cells). The pool of phages which bind to the desired substrate can then be used to generate retroviruses that can be used to infect mammalian cells. A large pool of phage can then be tested individually for the ability to elicit a phenotype. pMODis I is designed to allow display on the surface of phage and of mammalian cells. Additionally by passage through a specific host strain pMODis I can be used to direct secretion of displayed peptides from mammalian cells. pMODis II is an intracellular display vector. Both are created by the insertion of cassettes between the EcoRI and XhoI sites (destroying these sites) of p.Hygro.MaRX II. The design of the individual cassettes is as follows.

### pMODis I Cassette

The pMODis I cassette contains the following elements in order:

1. the beta-globin minimal splice donor site;

2. the pTAC promoter;

3. a synthetic ribosome binding site;

4. the pelB secretion signal;

5. the beta globin minimal splice acceptor site;

6. a mammalian secretion signal (e.g. from the V-J2-C region of the mouse Ig kappa-chain);

7. the minibody 61 residue peptide display vehicle sequence (Tramontano, J. Mol. Recognit. 7: 9-24 (1994));

8. an FRT recombinase site;

9. the 37 amino acid DAF-1 GPI anchor (see Rice et al., PNAS 89: 5467-5471 (1992));

10. an FRT recombinase site;

11. an amber stop codon;

12. the C-terminus of the gene III protein, amino acids 198-406; and,

13. non-amber stop codons.

In an amber suppressor strain and in the presence of helper phage, a gene III fusion protein is produced and displayed on the surface of the M13-type phage. This allows display of random peptide sequence cloned into one or both of the two constrained loops of the minibody to be displayed on the phage surface. Expression in packaging cells of MODis I genomic retroviral RNA allows removal of the bacterial promoter and secretion sequences by pre-mRNA splicing and causes translation in the mammalian cell to begin at the first methionine of the minibody sequence. Furthermore, in a mammalian cell, the amber codon would terminate translation prior to the gene III sequence creating a membrane-bound extracellular minibody that displays a random peptide sequence. The minibody could be converted to a secreted protein by passage through a FLP-expressing strain of bacteria. This would cause site-specific recombination at the FRT sites and deletion of the membrane anchor sequence.

### pMODis II Cassette

The pMODisII contains the following elements in order:

1. the beta-globin minimal splice donor site;

2. the pTAC promoter;

3. a synthetic ribosome binding site;

4. the pelB secretion signal;

5. the beta globin minimal splice acceptor site;

7. the thioredoxin peptide display vehicle sequence (Colas et al., Nature 380: 548-550 (1996));

11. an amber stop codon;

12. the c-terminus of the geneIII protein, amino acids 198-406; and,

13. non-amber stop codons.

This vector is designed for intracellular peptide display. As with pMODis I, the bacterial promoter and signal sequences are removed upon retrovirus production by pre-mRNA splicing.

Both of the pMODis vectors can also be used directly for peptide display in mammalian systems.

### Example 6

### Preparation of Libraries

The following example provides the methods for the construction of the libraries of the present invention.

### Construction of Sense Expression Libraries in p.Hygro.MaRX II-LI

### Preparation of the Library Vector as Follows

For preparation of the library vector, 10-20 μg of twice CsCl purified vector are digested with 5U/μg of EcoRI and XhoI for 90 min at 37° C. This digestion is directly loaded onto a 1% agarose gel (SeaKem GTG), and cut vector is separated by electrophoresis in TAE buffer. The vector band is excised following visualization by long-wave UV light. The cut vector is eluted from the agarose by electrophoresis in dialysis tubing. The vector is further purified by phenol/chloroform extraction and ethanol precipitation. It is expected that a vector which is suitable for library preparation can generate >5×10⁶/0.5 μg colonies with <10% background (insert-less) upon ligation with an EcoRI/XhoI digested test insert.

### Preparation of cDNA Libraries

cDNA synthesis begins with an RNA population that is >10-20 fold enriched (as compared to total RNA) for mRNA. First strand cDNA synthesis is accomplished by standard protocols using Superscript II reverse transcriptase. 5-me-dCTP replaces dCTP in the first strand synthesis reaction to block digestion of the newly-synthesized cDNA with XhoI. The first strand cDNA primer is as follows:

5′-GAG AGA GAG AGT CTC GAG TTT TTT TTT TTT TTT TTT-3′ (SEQ ID No. 23)

The first nine nucleotides are modified backbone (phosphorthioate) to prevent nuclease degradation of the XhoI site (CTCGAG). Other modifications to the backbone (e.g., p-ethoxy, Peptide-nucleic acid—PNA) would also serve. Synthesis is initiated by addition of reverse transcriptase in the presence of a saturating amount of the primer and following a controlled hybridization at 37° C. to prevent synthesis of long oligo dT tails.

Second strand synthesis is accomplished by E. coli DNA polymerase I in the presence of RNAse H and E. coli DNA ligase. Termini generated by second strand synthesis are made blunt by the action of T4 DNA polymerase.

Double stranded cDNAs are size fractionated by gel filtration chromatography on Biogel A50M as described by Soares (Soares et al., 1994, Proc. Natl. Acad. Sci. 91:9228-9232).

Size fractionated cDNAs are ligated to commercial EcoRI adapters (Stratagene), and then treated with XhoI to create cDNA fragments with EcoRI (5′) and XhoI (3′) ends. Unligated adapters are removed by chromatography on Sepharose CL4B (Pharmacia). The adapter-bearing cDNA is phosphorylated using polynucleotide kinase and is ligated using T4 DNA ligase to the EcoRI-XhoI digested library vector at 16° C. for up to two days (600 ng vector plus 250 ng insert in a volume of 10-20μl). The library is amplified by electroporation into ElectroMax DH12S (Gibco-BRL) which are plated on 100 150 mm LB+ampicillin+IPTG plates. Alternatively, the library may be amplified in liquid media containing ampicillin and IPTG (to select against non-recombinant clones). At a minimum a library of >5×10⁶ clones is required. This is routinely achieved using our protocols.

### Normalization of cDNA Libraries

We use two protocols for the normalization of cDNA libraries. Both are based upon those reported by Soares et al., in Proc. Natl. Acad. Sci. U.S.A. 91:9228-9232, 1994. This precise procedure has been used, but we have also developed a modified and streamlined using biotinylated oligonucleotides to reduce the number of steps.

### Rescue of Single Stranded DNA

The retroviral library in E. coli DH12S is grown in 100 ml of culture volume to mid-log phase and is then infected at an m.o.i of 10 with a helper phage (e.g. M13K07 or VCS-M13+). The culture is incubated for from 2 two 4 hours at 37° C. after which single stranded DNA is purified from the supernatant using standard protocols.

### Purification of the Single Stranded Library DNA

The DNA prepared as described above is a mixture containing single stranded library DNA, ssDNA from the helper phage and double stranded DNA from lysed bacteria in the culture.

The DNA mixture is first digested with XbaI that cuts only double-stranded DNA within the retroviral LTR. This mixture is then treated with Klenow DNA polymerase in the presence of dATP, dGTP, dCTP and Bio-16-dUTP. This treatment will incorporate a biotin residue on both ends of each fragment. The DNA population is then annealed to an excess of a 40-mer oligonucleotide that is complementary to the helper phage. This oligonucleotide carries a biotin residue at its 5′ terminus (C16-biotin, Peninsula Labs). The unincorporated nucleotides and single stranded, biotinylated oligonucleotides are removed by chromatography on sepharose CL-4B. The biotinylated DNA fragments and the oligo-bound helper phage DNA is removed from the population by incubation with magnetic-streptavidin beads (Dynal). This yields a cDNA population that is comprised essentially of the single stranded library.

Normalization of the Library

Normalization of the cDNA library is accomplished by reassociation kinetics (C₀t). The purified single stranded DNA is first annealed to a common primer. In our protocol this is a biotinylated oligo dT₁₈ primer while in the Soares protocol the primer is not biotinylated. This primer is extended by Klenow polymerase in the presence of a mixture of dNTPs and di-deoxyNTPs to synthesize fragments (average ˜200 nt in size) complementary to the 3′ end of our cDNA population. Again unincorporated primers and nucleotides are removed by chromatography on CL4B. The purified DNA is concentrated by ethanol precipitation.

For the reassociation kinetics reaction, 100-200 ng of purified, partly duplex DNA is resuspended in 2.5 μl of formamide and heated at 80° C. for several minutes. An excess (˜5 μg) of oligo dT25 is added to block interaction of the extension products (see above) with single stranded library though the oligo dT stretches that are present at the end of each clone. 0.5 μl of 0.5M NaCl is added along with 0.5 μl of 100 mM Tris-HCl, 100 mM EDTA, pH 8.0 and 0.5 μl water. The mixture is incubated at 42° C. for 12-24 hours to produce a C₀t of 5-20.

Re-annealed duplexes represent abundant clones which are removed from the mixture (following dilution in binding buffer) by incubation with magnetic streptavidin beads. The non-bound fraction represents the normalized library and is enriched for unique sequences. This single stranded library is concentrated by precipitation and is annealed to an excess of a vector primer that lies downstream of the XhoI cloning site (lacO primer). Extension of this primer with T4 DNA polymerase (or the like) creates partially double stranded circles which are used to transform electrocompetent DH12S bacteria to produce the normalized library.

The transformed population is used for preparation of high-quality DNA by standard protocols.

### Selection of Retroviral Sub-libraries Specific to a Given Location Within a Genome

Sublibraries that contain sequences derived from specific loci in a given genome can be selected from the single-stranded DNA prepared as above. Loci-specific DNA sequences that contain mapped, yet unknown genes can be obtained as sorted chromosomes or as fragments born on YAC or BAC vectors. These sequences are obtained in pure form or are purified by standard methods. Purified DNA is digested with a restriction enzyme with a four-based recognition sequence. A double stranded oligonucleotide is ligated to the ends of these fragments. Excess double stranded oligonucleotide is removed by column chromatography and the fragments are amplified by PCR with a biotinylated primer that corresponds to one strand of the double stranded oligonucleotide. This results in the production of a population of biotinylated DNA fragments that are derived from a specific genomic locus. This population is then annealed in the presence of appropriate competitive DNA sequences (e.g., yeast genomic DNA, highly repetitive human DNA) to single-stranded retroviral cDNA libraries prepared as above. cDNAs that are derived from the region of interest can then be purified using magnetic streptavidin beads and rescued in bacteria as described above. The resulting retroviral sub-library is greatly enriched for sequences that are contained on the original sorted chromosome, YAC, or BAC. The ability of sequences in this sub-library to give rise to a known phenotype can then be tested following packaging and infection of the appropriate cell type.

### Preparation of Unidirectional Antisense Libraries

Unidirectional antisense libraries are prepared essentially as described for the sense orientation libraries (see above). Exceptions are as follows:

First strand synthesis is accomplished using a modified backbone random primer that incorporates a restriction site. For our purposes we use the oligonucleotide:

5′-GCG GCG gga tcc gaa ttc nnn nnn nnn-3′ (SEQ ID No. 24)

As with sense orientation libraries, the first six nucleotides contain a modified backbone structure that makes them nuclease resistant.

Following second strand synthesis, the library DNA is blunt-ended and ligated to XhoI linkers. These have the following structure:

(SEQ ID No.25)
5′-TCTCTAGCTCGAGCAGTCAGTCAGGATG-3′
(SEQ ID No.26)

5′-ATAAGAGATCGAGCTCGTCAGTCAGTCCTAC-3′

Ligation of these linkers permits amplification of the library by PCR. In this case, the purified cDNA must be digested with both EcoRI and XhoI. Alternatively, commercially available XhoI adapters are ligated to the cDNA. In this case, the library cannot be amplified by PCR, and digestion of the linker-ligated cDNA is with EcoRI. Size selection of the cDNAs is accomplished by gel electrophoresis since the goal is to isolate fragments with an average size of 200-500 nucleotides. This isolated DNA is then ligated into the MaRX IIg (or IIg-VA or IIg-dccmv) as described above. Normalization is also accomplished as described for the sense expression libraries except that the primer used for extension of the library circles is derived from a combination of the vector (lacO site) and the polylinker since these clones have no oligo dT sequences. This also necessitated the addition during the re-annealing (C₀t) step of an excess of the non-biotinylated primer to suppress hybridization via primer sequences.

### Single Gene Unidirectional Antisense Libraries

Single-gene antisense libraries (for use in targeted functional knockouts) are prepared essentially as described above except that the template for first strand synthesis is a transcript produced from a cloned cDNA using a bacteriophage RNA polymerase (typically T3, T7 or SP6 polymerase). The second deviation is that is type of library is not normalized.

### Example 7

### Preparation of Virus and Infection and Recovery

The following example provides the necessary protocols for the preparation of the virus and infection of cells with the virus, in addition to recovery of the provirus.

### Transfection of Packaging Cells and Infection with Virus

1. Plate 6×10⁶ packaging cells/10 cm plate. Keep at 37° C. for overnight. Cells should be about 70-80% confluent.

2. Replace culture with fresh medium (10 ml). Incubate at 37° C. for 1-4 hours.

3. Prepare 2 ml of DNA ppt solution for each transfection in two eppendorf tubes: mix 15 μg DNA+X μl water to achieve 450 μl total volume; add 50 μl 2.5M CaCl₂/0.01M HEPES (pH 5.5); mix dropwisely to form DNA precipitates by adding 500 μl 2×BBS (50 mM BES, 280 mM NaCl, 1.5 mM Na₂HPO₄, pH 6.95) to DNA/CaCl₂ mix while gently bubbling in DNA/CaCl₂ mix with a pasture pipette; immediately and dropwisely add DNA precipitation solution to cells while gently swirling the plate (2 ml DNA precipitation solution/10 cm plate).

4. Incubate at 37° C. for over night.

5. Replace with fresh medium. (Option: at this step dexoamethasone and sodium butyrate can be added to medium at final concentrations of 1 μM and 500 μM, respectively. This increases the viral titer by 2-10 fold).

6. 32° C. incubation for 48 hours.

7. Collect virus supernatant and filter it through a 0.45 μM syringe filter unit. (Optionally, packaging cells can be eliminated by spinning the virus supernatant at 1K rpm for 5 minutes).

8. Dilute virus supernatant in fresh growth medium and add polybrene to a final concentration of 8 μg/ml. Add the mixture to cells.

9. Spin the plates at 1.8K rpm for 1 hour at room temperature.

10. 32° C. incubation for over night.

At this point, multiple infection cycles can be done by replacing the media on the producer cells and repeating steps 7-10 at 6 hour intervals.

11. Replace with fresh medium. 37° C. incubation.

12. Cells are analyzed or drug selection applied after 2 days.

### Proviral Excision and Recovery

### Structure of the Cre and CreT viruses

Excision of viral plasmids for reversion of phenotypes is accomplished using a virus which directs the expression of Cre recombinase from the LTR promoter. This virus was prepared by excision of the Cre sequence from pMM23 (see Qin et al., 1994, PNAS 91: 1706-1710) and insertion of that fragment into pBabe-puro. Derivatives with other markers have also been constructed. For replicative excision, a cassette that includes the coding sequence of large T antigen (from pAT-t (a T antigen clone that can encode large T but not small t)) fused to the IRES sequence from EMCV (derived from pCITE) was inserted downstream of the Cre sequence.

### Excision in vivo

Infect (as described above) MaRX virus-containing cells with pBABE-puro-Cre virus when cells are at 40-80% confluence in 10 cm plates using 8 ml virus (generated as described above)+2 ml medium+10 μl 8 mg/ml polybrene (1000×).

For reversion, the cells are maintained at 32° C. overnight and then transferred to 37° C. These cells are then selected for the presence of the Cre virus by incubation in selective media (e.g. containing puromycin). After one or two passages, the cells may be analyzed for loss of the phenotype.

For in vivo excision and recovery of the viral plasmid, cells are infected with either the Cre or the Cre-T virus and then incubated overnight at 32° C. Cells are subsequently transferred to 37° C. for an additional 6-24 hours. DNA is prepared and the proviral plasmid is recovered by one of the methods described below.

### Preparation of DNA for Affinity Recovery

For recovery of provirus by affinity purification, a 10 cm dish at confluence is lysed as described below. For provirus that has been excised in vivo, cells will have been treated as described above. For recovery of provirus following purification, infected cells at 80-100% confluence are used.

Lysis buffer is 10 mM Tris, pH 8.0; 150 mM NaCl; 10 mM EDTA; 1% SDS; 500 μg/ml protease K; and 120 μg/ml RNase A.

1. lyse cells in 10 ml of lysis buffer/10 cm dish;

2. incubate at 55° C. for 3 hours;

3. add an equal volume of phenol/chloroform, rotate 10 minutes, spin at top speed;

4. add ⅕ vol 8M KAc and 1 vol chloroform, rotate 10 minutes, spin at top speed;

5. add 2 volumes of ethanol and spool onto a glass rod;

6. Wash genomic DNA 3×in 70% ethanol;

7. AIR dry pellet and resuspend in TE.

### Preparation of LacI Affinity Beads

LacI beads for affinity purification are prepared in one of two ways. A procedure has been published for the preparation of magnetic beads bearing a lacd-Protein A fusion. These have been prepared exactly as described by Lundeberg et al. Genet. Anal. Tech. Appl 7: 47-52 (1990).

### Recovery of DNA on LacI Beads

Proviral DNA can be recovered on LacI beads prepared as described above. For recovery of provirus that is excised in vivo or for recovery of provirus for excision in vitro, DNA preparations must be slightly sheared to reduce viscosity. This can be accomplished by brief sonication, repeated passage though a narrow gauge needle or by nebulization.

1. 1-50 μg of DNA is diluted to 58 μl ddH₂O;

2. add 15 μl of 5×binding buffer;

3. pellet 60 μl lacI beads on magnetic concentrator;

4. remove the supernatant and resuspend in DNA solution;

5. rotate at 37° C. for 60 minutes;

6. pellet beads and wash 1×with 250 μl 1×binding buffer;

7. resuspend in 75 μl IPTG elution buffer plus 5 μl 25 mg/ml IPTG;

8. rotate at 37° C. for 30 minutes;

9. add 30 μg of glycogen and ethanol precipitate.

For provirus that has been excised in vivo, electroporate the recovered DNA into DH12S/trfA.

### For Excision/recircularization in Vitro

Excision/recircularization in vitro is accomplished in one of several ways. The DNA can be treated with commercially available Cre, Kw, or other recombinases according to the manufactures instructions. The recircularized plasmids can then be used to transform E. coli by electroporation. Alternatively, most of the MaRX derived vectors have unique rare-cutting restriction enzyme sites adjacent to the loxP sites. These enzymes (e.g. NotI in p.Hygro.MaRX II) can be used for digestion of the proviral DNA followed by recircularization using T4 DNA ligase to create a plasmid that can be both propagated in bacteria and used for the production of subsequent generations of retroviruses.

To significantly enhance the chance of recoverying the excised plasmids/proviruses, either the genomic DNA or the excised plasmid/provirus can be optionally amplified (such as PCR or Rolling Circle Amplification) before the recoverying step, such as electroporation or transformation into bacteria cells.

For example, in one experiment, rolling circle amplification (RCA) using the phi29 DNA polymerase was performed on a test MaRX plasmid. Even without resolving the amplification product with the Kw recombinase, the amplification product can be directly used for transformation/electroporation and yield bacterial transformants. However, upon Kw excision of the RCA amplification product, a much greater number of colonies were observed (generally 100 colonies without Kw excision and 3,000 to 9,000 with excision under the condition tested). This would indicate that: (a) RCA can efficiently amplify the excised provirus and/or plasmids; (b) the tandem arrays of MaRX vector that are produced by phi29 rolling circle amplification are available for excision by the recombinase; and (c) the resolved amplification product yields functional plasmids/proviruses competent for bacterial transformation.

In addition, RCA amplification of excised vector from genomic DNA can be performed with random hexamers with a similar increase in vector transformation rate. This result demonstrates that specific primers to the excised vector are not needed for this amplification, and random promers of a given length (6-mer, 8-mer, etc.) may be universally used for RCA amplification of the excised vector.

### Alternative Recovery Method: Hirt Extraction

Following in vivo excision, proviral plasmids can be recovered by the Hirt procedure (Hirt, B., J. Mol. Biol. 26: 365-369 (1967)). This can be used for the recovery of single clones but it is relatively inefficient and thus may be difficult to use for high-efficiency recovery of enriched sub-libraries.

1. following in vivo excision, wash cells twice with 10 ml of PBS;

2. add 3 ml of 0.6% SDS/10 mM EDTA (pH7.5)/10 cm plate. Incubate at RT for 15 minutes to lyse cells;

3. transfer lysate to a 15 ml tube with a scraper and a 1 ml pipette tip cut wide at end (to avoid shearing genomic DNA);

4. add 750 μl of SM NaCl. Mix by gently inverting the tube;

5. incubate at 4° C. for more than 8 hours;

6. spin at 15K rpm for 20 minutes in JA20 at 4° C. and save supernatant;

7. extract with 1 vol. of phenol/chloroform and then with chloroform;

8. precipitate DNA by adding 20 μg of glycogen and 2.5 vol of EtOH;

9. dissolve DNA in 200 μl of water. Extract with 1 vol. of phenol/chloroform and then with chloroform;

10. dissolve DNA in 10 μl of water;

11. electroporate DNA into DH12S/trfA (see below):

5 μl of recovered DNA+50 μl of cells on ice;

1.8 kV×25 uFD×200Ω in 0.1 cm cuvette (BioRAD);

add 1 ml of 2XYT;

37° C. recover for 1 hour;

plate 200 μl on LB (½ NaCl, pH 7.5)-zeocine (25 μg/ml)

37° C. for over night.

This procedure generally yields several hundred proviral colonies.

### Proviral Host Strain: DH12S/trfA

The RK2 replication origin (Oriᵥ) requires a replication protein, trfA for function. Otherwise it is a silent DNA element thus allowing it to co-exist with a pUC replication origin on the same plasmid. The excised provirus depends on the RK2 origin for replication and thus for propagation of this plasmid, trfA must be provided in trans. Thus, a trfA-helper strain has been constructed using DH12S as a founder strain. Several characteristics of DH12S prompted its choice for construction of the helper strain. Firstly, it is defective in the restriction system that causes degradation of methylated DNA. Secondly, it is recA, recBC and will thus more stably maintain plasmids. Thirdly, it can be used for the production of single-stranded DNA. Finally, DH12S can give rise to high-efficiency electrocompetent cells.

Since Oriᵥ-based plasmids are generally maintained at low copy number, a copy-up mutant of the replication protein (trfA-267L; Blasina, 1996. Copy-up mutants of the plasmid RK2 replication initiation protein are defective in coupling RK2 replication origins. Proc. Natl. Acad. Sci. U.S.A. 93: 3559-3564 (1996)) was used for the preparation of the strain. This mutant was first cloned into pJEH118 (Fabry et al., 1988, FEBS Letters 237: 213-217) to place it under the control of the pTac promoter. This allows inducible, high level expression which helps to offset the loss in expression levels that occur as trfA integrated into the chromosome at single. A kanamycin resistance marker was then cloned downstream of the trfA cassette. The entire cassette was excised and inserted into a lambda phage vector (lambda-NM540) which was packaged in vitro and used for the preparation of a DH12S lysogen. Several lysogens were tested for the ability to propagate Oriᵥ plasmids and one was chosen as DH12S/trfA.

### Example 8

### Production of Packaging Cell Lines

### Creation of Cassettes that Provide Viral Functions

Three viral functions are provided in trans by packaging cell lines. These are gag, pol and env. In general, either all three are provided by a single cassette or the gag/pol and env functions are separated onto two cassettes. To create directly selectable cassettes that can provide viral functions in trans, genes encoding viral proteins have been transferred from a helper plasmid that consists of a defective provirus (psi⁻e; Mann et al., Cell 33: 153-9 (1983)) to pBluescript in two formats.

### Single Gene Helper Cassettes

To produce an ecotropic single gene helper cassette, the XhoI-ClaI fragment was purified from psi⁻e and transferred to a similarly digested pBS-SK+ to create pBS+psixc. The end of the envelope gene was reformed by adding a ˜100 nt PCR product which spanned the sequences from the ClaI site to the stop codon of the envelope protein. This procedure also added a unique EcoRI site to the 3′ end of the helper cassette. The PCR product was inserted into pBS-psiXC following digestion of both DNAs with EcoRI and ClaI. The resultant plasmid was pBS-psi-XE. The 5′ end of the helper cassette was created by insertion of a PCR product which spanned from the retroviral splice donor site at the 5′ end of the packaging signal to the unique XhoI site of MoMuLv. This PCR product was inserted into an XhoI digested pBS-psiXE in such a way that a unique SspI site was present at the 5′ end of the cassette. This formed pBS-psiCOMP. This helper cassette could encode gag, pol and env, but lacked the LTR elements and tRNA primer binding sequences necessary to produce a replication competent virus. To allow direct selections for viral functions, a tri-cistronic message cassette was created by inserting two tandem IRES-linked markers downstream from the end of the envelope sequence. In this case the cassette contained an EMCV IRES linked to human CD8 protein (a cell surface marker) linked to another EMCV IRES linked to the hygromycin resistance gene. This was inserted from EcoRI to NotI in pBS-psiCOMP to form pBS-psiCD8H. The cassette from this plasmid can be inserted into any expression vehicle following excision by SspI and NotI.

Separation of helper functions onto two cassettes was accomplished by creating deletions of pBS-psiCOMP. The env function was isolated by digestion of pBS-psiXE with XhoI and XbaI followed by insertion of a linker sequence that reformed both restriction sites. Removal of env from pBS-psiCOMP was accomplished by digestion with HpaI and EcoRI followed by ligation with a synthetic fragment that repaired the 3′ end of pol and that reformed both the HpaI and EcoRI restriction sites. The single cassette amphotropic envelope (Ott, D. E. et al., J. Virol. 64, 757-766 (1990)) was formed by PCR followed by insertion into pBS. Each of these plasmids was used to generate a tri-cistronic helper cassette. Each envelope plasmid received the CD8-hygromycin cassette described above. The gag/pol plasmid received either of two cassettes. One consisted of an EMCV IRES linked to the gene encoding a cytoplasmic domain defective CD4 (another cell surface marker) linked to an EMCV IRES linked to the gene for histidinol resistance. The second cassette consisted of an EMCV IRES linked to the gene encoding green fluorescent protein linked to and FDV IRES linked to the gene encoding puromycin resistance.

Since all of these tricistronic cassettes are used similarly to introduce packaging functions into cells, introduction of the single gene helper cassette will be described. Introduction of the separated helper functions simply requires additional quantitative and qualitative selection steps.

### Expression Vehicles

The helper cassettes described above must be functionally linked to sequences that promote expression in mammalian cells. These constructs can then be introduced into cell lines to create a functional packaging system. In general two options are available. The single helper cassette can be cloned in functional association with a strong promoter (e.g. CMV) in a plasmid that can replicate in the presence of SV40 T antigen. This allows amplification of the plasmid episomally. In some cases this is followed by high copy integration into the genome. Such a plasmid can also be used in the absence of SV40 T-antigen to achieve somewhat lower copy numbers. For this purpose the single helper cassette has be inserted into pcDNA3 (Invitrogen). Alternatively, the helper cassette can be placed in association with a strong promoter on a vector that replicates as a stable episome. Two such systems are in common use. The first is based upon Epstein Barr Virus. EBV-based vectors replicate via oriP which requires EBNA for function. A particularly useful vector has been produced by Invitrogen (pCEP-4). This vector has been modified to remove the hygromycin resistance cassette and the helper cassette has been inserted downstream of the CMV promoter. Upon transfection into our chosen host cell line, this vector can achieve stable copy numbers of >20/cell. The final choice is a set of vectors based upon bovine papilloma virus. Unfortunately, these vectors will not replicate in our host cell of choice and we must therefore obtain modified BPV vectors in which viral functions are expressed from a constitutive promoter that functions in our chosen cell type. These modified BPV vectors can achieve copy numbers that range from 100-1000/cell.

### Cell for the Generation of Packaging Cell Lines

Human 293 cells have been chosen for the generation of packaging cell lines. These cells can support replication from SV40-based systems and EBV based systems. These can also be used for the high copy number, modified BPV systems. In particular, a subline of human 293 cells (293T) shows extremely high transfection efficiencies (this is critical for the production of high-complexity libraries) and contains a temperature sensitive SV40 large T antigen that can support conditional replication of SV40-based vectors.

### Selection of Packaging Cell Clones

Human 293T cells will be transfected with either the single helper plasmid or the two separate helper plasmids in the vectors described above. Transfected cells will be placed in selective media containing standard concentrations of hygromycin (75 μg/ml) or hygromycin plus puromycin (1.5 μg/ml). Following successful selection of stably transfected clones, high-expressing cells will be selected by FACS analysis following staining with antibodies directed against the cell surface markers or by direct detection of gfp. The 5% of clones which display the highest expression levels will be recovered and plated again in selective media. Cells will be passed into a media containing a 50% higher concentration of each drug and the 5% of surviving cells which display the highest marker expression will be passed through another round of this procedure. At each round, levels of elaborated reverse transcriptase and transfection rates are assessed. After several rounds, at a time at which subsequent rounds fail to increase reverse transcriptase expression or at which high drug concentrations result in a reduced transfection rate, single cell clones will be chosen and analyzed for the ability to produce high titer virus. The ability to enforce direct selection for the viral helper cassettes should allow not only selection of the most efficient packaging cells but should also allow for continuous selection for maintenance of high efficiency packaging function.

It is recommended that during initial set up, the user also optimize the system by using a retroviral vector expressing an easily assayable marker such as lacZ or a cell surface protein. During optimization, one should check for transfection frequency of the producer clone and test infection rate of target cells. Tests for transfection and infection frequencies using a βgal-based system or the like can be readily measured by βgal staining or FACS staining for βgal activity. Only when the user is satisfied with the transfection conditions and infection rates should s/he proceed to using vectors with no readily assayable marker. It should be possible to scale up the protocols.

Moreover, in certain instances the initial plating of the cells may be the most important step in successfully obtaining high retroviral titers. It is extremely important that the cells are not overly clumped and are at the correct density. Unlike NIH3T3-derived cell lines, the 293-derived packaging cell lines and the like do not readily form well-spread monolayers. Instead, they tend to clump before confluence, and if the clumping is excessive, the cells will never reach confluence during the 48-72 hour period following transfection. In order to prevent clumping, it is essential that the cells are extremely healthy prior to plating. If they are overconfluent, it may be necessary to split them 1:2 or 1:3 for several passages prior to plating for transfection. In addition, the cells are much less adherent than murine fibroblasts and should be handled very gently when washing and changing medium. For consistency, it is important to count the cells rather than estimating the split. The above cell number is optimized for MFG-lacZ. Expression of other inserts may be detrimental to the growth of the cells. This effect may be noted by failure of the packaging cell line to reach confluence by 48-72 hours post-transfection. If this occurs, it may be necessary to plate more cells prior to transfection.

Further more, the addition of chloroquine to the medium appears can increase retroviral titer. This effect is presumably due to the lysosomal neutralizing activity of the chloroquine. In many instances, it is important that the length of chloroquine treatment does not exceed about 12 hours. Longer periods of chloroquine treatment have a toxic effect on the cells causing a decrease in retroviral titers. For purposes where achieving maximal retroviral titer is not necessary, such as when comparing the relative titers of different constructs, it may be preferable to omit chloroquine treatment. If chloroquine is not used, it is unnecessary to change the medium prior to transfection.

To further illustrate an exemplary embodiment, when the retroviral supernatant is ready for harvesting, gently remove the supernatant and either filter through a 45 μM filter or centrifuge×5 min at 500×g at 4° C. to remove living cells. If the retroviral supernatant is to be used within several hours, keep on ice until it is used.

### Example 9

### pEHRE-BASED Packaging Cell Lines

Utilizing techniques as described in the Example presented in Section 13, above, the pEHRE family of vectors has been used to successfully create packaging cell lines for the production of retroviruses following either transient or stable transfection with replication-deficient retroviral vectors.

Specifically, two ecotropic 293T based packaging lines, referred to herein as LinX I and LinX II have been created.

In LinX I, helper functions are supplied on a pEHRE vector containing a single expression cassette that encodes gag, pol and env. In LinX II, the gag/pol and env functions are supplied on separate pEHRE vectors. Both cell lines produce virus with a titer in of 10⁶ pfu/ml as measured on NIH3T3 cells. In this respect LinX I and LinX II are equivalent to the best available packaging lines. However LinX I and LinX II do have two additional unusual and beneficial characteristics.

First, the initial, drug-selected pool from which the packaging cell lines were derived was able to package virus with an efficiency that is nearly equivalent to the clone that was finally selected as the packaging cell line. This is in contrast to cell lines constructed by standard procedures in which the efficiency of the transfected pool is 2-3 logs lower than that of a cell line that is eventually derived from the analysis of hundreds of cell clones. The ability of the pEHRE multi-copy episomal system to deliver viral helper functions, therefore makes it ideal for the rapid construction of special-purpose packaging lines (e.g. cell lines with alternative or mutant gag or envelope proteins).

The second unusual characteristic of the LinX I and LinX II cell lines is that the cells exhibit a remarkably stable ability to produce high-titer virus. The ability of standard packaging cell lines (e.g. Bosc) to produce high titer virus decays very rapidly. For example, viral titers can decrease by more than one log per month. In contrast, LinX cells have been maintained for more than six months in culture without a detectable loss in viral titers.

This stability may result from a combination of two factors. First, the pEHRE episome is highly stable both in structure and in copy number. Second, the viral helper functions are present on these episomes as one segment of a polycistronic mRNA comprising the helper function and a drug resistance marker. Selection for the drug marker, therefore, allows direct selection for the mRNA encoding the helper function.

### Example 10

### Target Antisense Expression—Derivation of a Functional Knockout

Single gene antisense libraries in the MaRX IIg vectors can be used to created targeted functional knockouts of individual genes. This can be accomplished irrespective of prior knowledge of the phenotype of the knockout by creating an indirect selection for loss of gene function. This is accomplished by creating a quantifiable marker that serves to report the levels of expression of a particular gene. This can be created in any of a number of ways as described in the text of the application. The most straightforward is to create a fusion protein and this will be the example given.

The coding sequence of the protein of interest is fused to a reporter, in this case, the green fluorescent protein. This fusion should be prepared so that the 5′ and 3′ untranslated sequences are present in the construct. The entire cassette, including untranslated sequences is placed within a retroviral vector that promotes constitutive expression. Inducible vectors can also be used if expression of the fusion protein is deleterious. This vector is inserted into cells of a species distinct from the species from which the knock-out target is derived. For example, mink cells would make a reasonable screening host for human proteins. A population of cells showing uniform fluorescence is selected by single-cell cloning or by FACS. A single-gene, unidirectional antisense library is constructed from the transcript encoding the target gene (see above) in one of the MaRX IIg vectors. This library is used to infect cells that express the fluorescent fusion. By FACS sorting, cells which no longer express the fusion are identified. These are cloned as single cells. A subset of these will express antisense transcripts which effectively inhibit expression of the fluorescent fusion protein, and a subset will simply have lost fusion protein expression independent of an introduced antisense (revertants). Effective antisense can be distinguished from revertants by the ability of Cre recombinase to rescue fluorescent protein expression. Cell clones in which fluorescence is rescued by Cre will serve as a source for the recovery of viruses carrying antisense fragments which can be used to create functional knockouts in any desired cell line. It should be noted that this procedure is quantitative and qualitative; by FACS sorting, the most effective fragments can be identified as those able to quantitatively reduce fluorescence to the greatest extent. Furthermore, by replacing the CMV promoter in the MaRX IIg and MaRX IIg-dccmv with an inducible promoter (in combination with a self-inactivating LTR), conditional knockouts can be created.

### Example 11

### Activation of the Telomerase Enzyme

Telomerase is an almost universal marker for tumorigenesis. Activity is, however, absent in normal cells. Activity can be induced in a subset of normal cells (e.g., epithelial cells and keratinocytes) by introduction of the E6 protein from KPV-16. This induction is independent of the ability of E6 to direct degradation of p53. In order to investigate the processed that lead to the induction of telomerase in tumors, we have devised an in vitro screen for genes that can induce telomerase activity in normal human mammary epithelial cells (HMEC).

Pools of cDNAs comprising from 100-100 clones each (either in the sense orientation or in the antisense orientation in the MaRX IIg vector series) are introduced into HMEC cells. These are selected for expression of cDNA and then used to prepare lysates for the assay of telomerase activity. Cell lysates are tested using a highly sensitive telomerase assay which is capable of detecting two telomerase-positive cells among 20,000 telomerase-negative cells. Those pools which upon infection cause the induction of telomerase activity in HMEC cells are subdivided into smaller pools. Sub-pools are again used for the infection of BAMEC cells which are subsequently assayed for telomerase activity. Successive rounds of this procedure can identify an individual clone that acts as an inducer of the telomerase enzyme.

Such a clone could represent a direct regulator of the enzyme itself or of the expression of a component of the enzyme. Alternatively, such a clone could act as a regulator of cell mortality. Changes induced by the expression of such a clone could induce the telomerase enzyme as only one aspect of a more global change in cellular behavior.

### Example 12

### Secretion Screening

The retroviral and pEHRE vectors of the invention can be utilized in conjunction with secretion trapping constructs to identity nucleotide sequences which encode secreted proteins. Such identification schemes can serve a variety of purposes. For example, because secreted proteins are often useful as therapeutics, their identification can then be followed by additional biological screens as part of a method for identifying novel therapeutic agents. Additionally, identification of secreted proteins differentially expressed in a disorder such as, for example, cancer, can serve as convenient blood borne marker for diagnosing the presence of the disorder. Still further, identification of secreted proteins can act as a subfractionation which may make possible detection of an extremely rare sequence or event, which would go undetected if a sequence was not first enriched from a library in such a fashion.

Nucleotide sequences to be tested are introduced into the cloning site of a secretion trapping retroviral or pEHRE vector.

A plurality of secretion screening vectors containing nucleotide inserts, making up a secretion screening library, can be produced and screened simultaneously. Unidirectional random priming strategies, as described above for the production of unidirectional sense and antisense libraries can be used to produce such libraries.

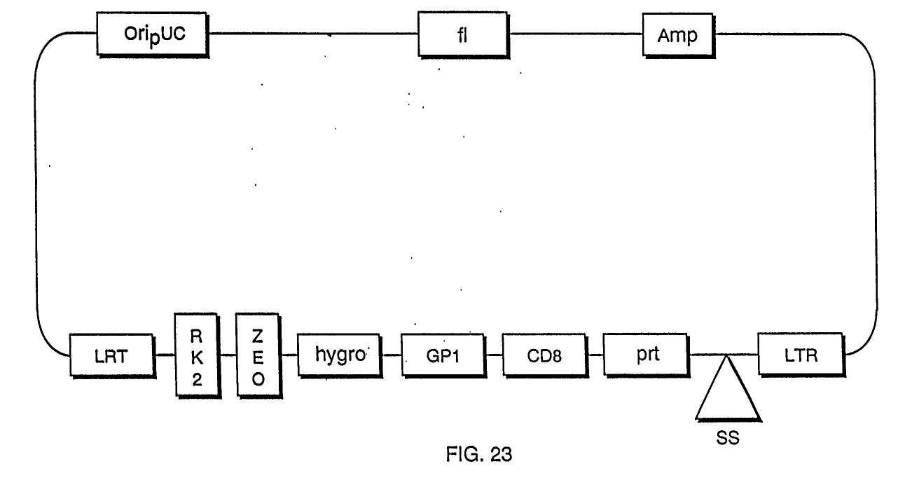

In one embodiment, a secretion trapping cassette comprises from 5′ to 3′: a transcriptional regulatory sequence, a polylinker, a protease coding sequence, flanked by protease recognition sites, a cell surface marker coding sequence (lacking a signal sequence) and a cell surface membrane anchoring sequence (preferably one whose anchoring activity is dependent upon the presence of a signal sequence, such that background is reduced, as described below), an IRES and a selectable marker. A representative retroviral secretion screening vector is depicted in FIG. 23.

Cell surface markers can include, but are not limited to, CD4, CD8 or CD20 marker, in addition to any synthetic or foreign cell surface marker. Protease and protease recognition sequences can include, but are not limited to any retroviral protease sequences, HIV, MuLv, RSV or ASV protease sequences.

Nucleotide sequences to be tested are introduced into the polylinker. The vectors containing such sequences are transfected or transformed, depending on the vector used, into cells. The vectors' selectable markers are used to select for cells which has taken up vectors.

Sequences coding for secreted proteins (i.e., sequences which code for signal sequences) are then identified by determining which of these cells exhibit the fusion protein cell surface marker. This is because the marker will only end up transported to and anchored on the cell surface if the fusion protein it becomes a part of contains a signal sequence.

In order to reduce extraneous background cell surface targeting, the membrane targeting portion of the fusion protein should, preferably, be one whose targeting activity is dependent on the presence of a signal sequence. For example, the GPI membrane anchoring/targeting sequence only becomes tethered on the cell membrane if it first goes through the cell's endoplasmic reticulum (ER). The presence of a signaling sequence, targets a protein to the ER, then serves to “activate” GPI's membrane tethering capability.

The protease element of the fusion protein can, in general, be used to create multiple functional units from one polypeptide translational unit. The protease element of the fusion protein is, in this specific instance, used to ease the identification of those cells which exhibit the cell surface marker. Specifically, by placing the protease and protease recognition sequence at the appropriate position along the fusion protein, the protease's activation and self cleavage serve to make the cell surface marker readily available to cell surface antibodies. Standard antibody-related isolation techniques such as FACS or magnetic bead isolation techniques can be utilized.

Utilizing the FIG. 23 vector, a single positive cell in one million was successfully purified to approximately 40% purity in only 4 rounds of screening.

### Deposit of Microorganisms

E. coli strain XL-1 carrying plasmid pMaRX II, was deposited on Sep. 20, 1996 with the Agricultural Research Service Culture Collection (NRRL), under the provisions of the Budapest Treaty on the International Recognition of the Deposit of Microorganisms for the Purposes of Patent Procedures and assigned accession number B-21625.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

### Equivalents

Those skilled in the art will recognize, or be able to ascertain using no more than routine experimentation, many equivalents of the specific embodiments of the invention described herein. Such equivalents are intended to be encompassed by the following claims.

We claim:

1. A method of amplifying a target nucleic acid sequence in a target cell genome comprising:
(A) excising the target nucleic acid sequence from the target cell genome;
(B) amplifying the excised target nucleic acid sequence by rolling circle amplification with a polymerase to generate a tandem series of the target nucleic acid sequence; and,
(C) excising the target nucleic acid sequence from the tandem series to produce individual target nucleic acid sequences;
thereby amplifying the target nucleic acid sequence in the target cell genome.

2. The method of claim 1, further comprising amplifying the entire target cell genome prior to excising the target nucleic acid sequence from the target cell genome.

3. The method of claim 1 or 2, wherein the target nucleic acid sequence is a replication-deficient retroviral vector.

4. The method of claim 1 or 2, wherein the target cell genome is a mammalian genome.

5. The method of claim 2, wherein the entire target cell genome is amplified by whole genome amplification.

6. The method of claim 2, wherein the entire target cell genome is in a cell and wherein the entire target cell genome is amplified by mitotic division of said cell.

7. The method of claim 1 or 2, wherein excision of the target nucleic acid from the target cell genome is effected by recombination with a recombinase.

8. The method of claim 7, wherein the recombinase is a Kw recombinase.

9. The method of claim 1 or 2, wherein excision of the target nucleic acid from the target cell genome is effected by restriction with a restriction endonuclease that cuts within the target nucleic acid sequence to release a target nucleic acid sequence with ligatable ends.

10. The method of claim 9, further comprising ligating said ends.

11. The method of claim 9, wherein the endonuclease is an HO endonuclease.

12. The method of claim 1 or 2, wherein the target nucleic acid sequence is amplified by a factor of at least about 10.

13. The method of claim 12, wherein the target nucleic acid sequence is amplified by a factor of at least about 100.

14. The method of claim 13, wherein the target nucleic acid sequence is amplified by a factor of at least about 1,000.

15. The method of claim 14, wherein the target nucleic acid sequence is amplified by a factor of at least about 10,000.

16. The method of claim 15, wherein the target nucleic acid sequence is amplified by a factor of at least about 100,000.

17. The method of claim 16, wherein the target nucleic acid sequence is amplified by a factor of at least about 1,000,000.

18. The method of claim 1 or 2, wherein the target nucleic acid sequence excised from the target cell genome is amplified by rolling circle amplification using a Phi 29 DNA polymerase.

19. The method of claim 1 or 2, wherein the target nucleic acid sequence excised from the target cell genome is amplified by rolling circle amplification using a DNA polymerase derived from a double stranded DNA virus.

20. The method of claim 1 or 2, wherein the target nucleic acid sequence is a retroviral vector having long terminal repeat ends.

21. A method of transferring a mixture of target nucleic acid sequences from a first vector system to a second vector system comprising:
(A) providing a first library of target nucleic acid sequences in a first vector system;
(B) excising the target nucleic acid sequences from the first vector system;
(C) amplifying the excised target nucleic acid sequences by rolling circle amplification with a polymerase to generate tandem series of the target nucleic acid sequences;
(D) excising the target nucleic acid sequences from the amplified tandem series of the target nucleic acid sequences to generate a mixture of amplified target nucleic acid sequences with free ends;
(E) providing a second vector system compatible with said free ends; and
(F) ligating the free ends of the amplified and/or excised target nucleic acid sequences to the second vector system, thereby transferring a mixture of target nucleic acid sequence form the first vector system to the second vector system.

22. The method of claim 21, wherein the first vector system is a replication-deficient retroviral vector.

23. The method of claim 21, wherein the target nucleic acid sequence excised from the first vector system is amplified by rolling circle amplification using a Phi 29 DNA polymerase.

24. The method of claim 21, wherein the target nucleic acid sequence excised from the first vector system is amplified by rolling circle amplification using a DNA polymerase derived from a double stranded DNA virus.

25. The method of claim 1 or 2, wherein the target nucleic acid sequence is a retroviral vector having long terminal repeat ends.

26. A method of converting a mixture of partial cDNA target nucleic acid sequences to a mixture of cognate full-length cDNA target nucleic acid sequences comprising:
(A) providing a first library of partial cDNA target nucleic acid sequences in a first vector system;
(B) excising the partial cDNA target nucleic acid sequences from the first vector system;
(C) amplifying the excised target nucleic acid sequences by rolling circle amplification to generate a hybrid capture probe mixture;
(D) contacting the hybrid capture probe mixture with a second library of full-length single-stranded cDNA target nucleic acid sequences to select full-length single-stranded cDNA sequences which correspond to the partial cDNA target nucleic acid sequences of said first library; and,
(E) releasing the selected full-length single-stranded cDNA sequences from the second library;
thereby converting a mixture of partial cDNA target nucleic acid sequences to a mixture of cognate full-length cDNA target nucleic acid sequences.

27. A method of converting a mixture of cDNA target nucleic acid sequences to a mixture of cognate genomic target nucleic acid sequences comprising:
(A) providing a first library of cDNA target nucleic acid sequences in a first vector system;
(B) excising the cDNA target nucleic acid sequences from the first vector system;
(C) amplifying the excised cDNA target nucleic acid sequences by rolling circle amplification to generate a hybrid capture probe mixture;
(D) contacting the hybrid capture probe mixture with a second library of cognate genomic target nucleic acid sequences to select cognate genomic sequences which correspond to the cDNA target nucleic acid sequences of said first library; and,
(E) releasing the selected cognate genomic target nucleic acid sequences from the second library;
thereby converting a mixture of cDNA target nucleic acid sequences to a mixture of cognate genomic target nucleic acid sequences.

28. A method of amplifying polynucleotides, comprising amplifying the polynucleotides by rolling circle amplification with a polymerase and random primers.

29. The method of claim 28, wherein the polynucleotide is genomic DNA (gDNA).

30. The method of claim 29, wherein the genomic DNA is whole genomic DNA isolated from cells.

31. The method of claim 28, wherein the polynucleotide is cDNA.

32. The method of claim 31, wherein the cDNA is reverse transcribed from RNA.

33. The method of claim 31, further comprising a step of size-fractionating the resulting amplified cDNA to select for substantially full-length cDNA.

34. The method of claim 28, wherein the random primers are random hexamers.

35. The method of claim 28, wherein the polymerase is Phi 29 DNA polymerase.

36. A method to clone a DNA fragment from a single cell, comprising:
(A) isolating genomic DNA containing the DNA fragment;
(B) amplifying the isolated genomic DNA by rolling circle amplification using a polymerase and primers;
(C) excising the DNA fragment from the amplified genomic DNA; and,
(D) cloning the excised DNA fragment.

37. The method of claim 36, wherein the DNA fragment is a provirus.

38. The method of claim 36, wherein the primers are random hexamers.

39. The method of claim 36, wherein the polymerase is Phi 29 DNA polymerase.

40. The method of claim 36, wherein the DNA fragments are excised by restriction endonuclease.

41. The method of claim 36, further comprising colonal expansion of the single cell prior to isolating genomic DNA.

42. The method of claim 36, wherein the DNA fragments are flanked by recombinase recognition sites and the DNA fragments are excised by a corresponding recombinase of a site specific recombinase system.

43. The method of claim 42, wherein the recombinase is Kw recombinase.

44. The method of claim 42, wherein the site specific recombinase system is selected from the group consisting of: the Cre/lox system of bacteriophage P1, the FLP/FRT system of yeast, the Gin recombinase of phage Mu, the Pin recombinase of E. coli, and the R/RS system of the pSR1 plasmid.

45. The method of claim 36, further comprising a step to enrich the excised DNA fragment prior to cloning.

46. A kit for amplifying a polynucleotide, comprising:
(A) a DNA polymerase suitable for rolling circle amplification;
(B) a reaction buffer for carrying out rolling circle amplification using the DNA polymerase;
(C) a mixture of random hexamer oligonucleotides.

47. The kit of claim 46, further comprising at least one of the components selected from the group consisting of: an instruction for using the kit; a control polynucleotide; a stock solution of dNTP mixtures or each of the four deoxynucleotides (dATP, dGTP, dCTP and dTTP).

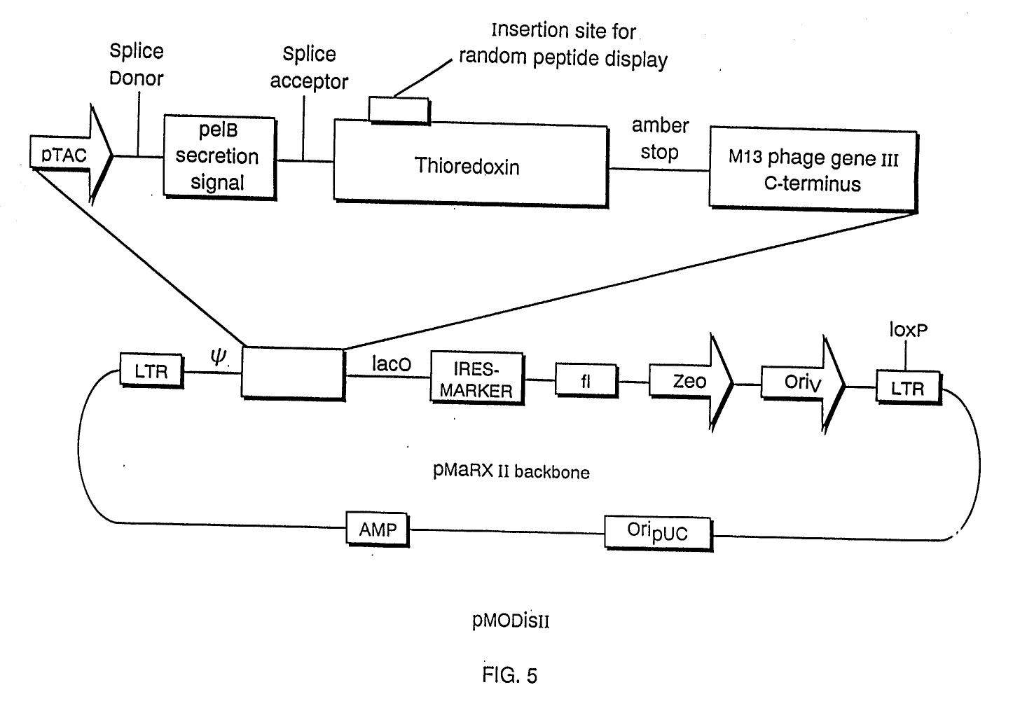

48. A polynucleotide sequence comprising a vector as shown in any one of FIGS. 1-23, or derivative thereof.

49. A polynucleotide sequence comprising a reunification vector as shown in FIG. 25 or derivative thereof.

50. A library comprising any one of the vector of claim 48 or 49.


SEQUENCE LISTING (26 sequences)

SEQ ID NO: 1 - DNA, 78 bp, Artificial Sequence
  Linker comprising a loxP site
ctagcataacttcgtataatgtatgctatacgaagttatgtattgaagcatattacatacgatatgcttcaatagatc

SEQ ID NO: 2 - DNA, 88 bp, Artificial Sequence
  Upper strand of polylinker
ggatccgtaaaacgacggccagtttaattaagaattcgttaacgcatgcctcgagtgtggaattgtgagcggataacaatttgtcgac

SEQ ID NO: 3 - DNA, 486 bp, Artificial Sequence
  Upper strand of PCR fragment
gtcgacaggcctcggacctgcagcacgtgttgacaattaatcatcggcatagtatatcggcatagtataatacgactcactataggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgattccggatttatcgat

SEQ ID NO: 4 - DNA, 359 bp, Artificial Sequence
  Top strand of PCR fragment
tccggacgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccggagatctatcgat

SEQ ID NO: 5 - DNA, 472 bp, Artificial Sequence
  f1 fragment
gcggccgcgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaagcggccgc

SEQ ID NO: 6 - DNA, 15 bp, Artificial Sequence
  Synthetic fragment
gatctttaattaaat

SEQ ID NO: 7 - DNA, 13 bp, Artificial Sequence
  Synthetic fragment
cgatttaattaaa

SEQ ID NO: 8 - DNA, 13 bp, Artificial Sequence
  Synthetic fragment
ccgggtttaaact

SEQ ID NO: 9 - DNA, 13 bp, Artificial Sequence
  Synthetic fragment
ccggagtttaaac

SEQ ID NO: 10 - DNA, 17 bp, Artificial Sequence
  NotI linker
ctagatgcggccgctag

SEQ ID NO: 11 - DNA, 17 bp, Artificial sequence
  NotI linker
ctagctagcggccgcat

SEQ ID NO: 12 - DNA, 100 bp, Artificial Sequence
  PCR fragment comprising the SV40 origin
ggggtttaaacgactaattttttttatttatgcagaggccgaggccgcctctgcctctgagctattccagaagtagtgaggaggcttttttggaggcccc

SEQ ID NO: 13 - DNA, 20 bp, Artificial Sequence
  Synthetic polylinker
gatcgttaattaacaattgg

SEQ ID NO: 14 - DNA, 20 bp, Artificial Sequence
  Synthetic polylinker
tcgaccaattgttaattaac

SEQ ID NO: 15 - DNA, 26 bp, Artificial Sequence
  Primer
gggagatctacggtaaatggcccgcc

SEQ ID NO: 16 - DNA, 43 bp, Artificial Sequence
  Primer
cccatcgatttaattaagtttaaacgggccctctaggctcgag

SEQ ID NO: 17 - DNA, 26 bp, Artificial Sequence
  Primer
ggggctagcacggtaaatggcccgcc

SEQ ID NO: 18 - DNA, 43 bp, Artificial Sequence
  Primer
gggtctagattaattaagtttaaacggccaaaaaagcttgcgc

SEQ ID NO: 19 - DNA, 33 bp, Artificial Sequence
  Primer
ggggctagcctaggaccgtgcaaaatgagagcc

SEQ ID NO: 20 - DNA, 43 bp, Artificial Sequence; identical to SEQ ID NO: 18
  Primer

SEQ ID NO: 21 - DNA, 100 bp, Artificial Sequence
  Polylinker top strand
agatcttgtggaattgtgagcggataacaatttggatccgtaaaacgacggccagtttaattaagaattcgttaacgcatgcctcgaggtcgacatcgat

SEQ ID NO: 22 - DNA, 70 bp, Artificial Sequence
  3′ LTR deletion
taactgagaatagagaagttcagatcaaggtcaggagatccctgagcccacaacccctcactcggggcgc

SEQ ID NO: 23 - DNA, 36 bp, Artificial Sequence
  Primer
gagagagagagtctcgagtttttttttttttttttt

SEQ ID NO: 24 - DNA, 27 bp, Artificial Sequence
  Primer
gcggcgggatccgaattcnnnnnnnnn

SEQ ID NO: 25 - DNA, 28 bp, Artificial Sequence
  XhoI linker
tctctagctcgagcagtcagtcaggatg

SEQ ID NO: 26 - DNA, 31 bp, Artificial Sequence
  XhoI linker
ataagagatcgagctcgtcagtcagtcctac